(19) United States
(12) Patent Application Publication
(10) Pub. No.: US 2026/0202743 A1
(43) Pub. Date: Jul. 16, 2026

(54) RESIST COMPOSITION, RESIST PATTERN FORMATION METHOD, COMPOUND, AND ACID DIFFUSION CONTROL AGENT

(71) Applicant: Tokyo Ohka Kogyo Co., Ltd., Kawasaki-shi (JP)

(72) Inventor: Akiya Fujino, Kawasaki-shi (JP)

(21) Appl. No.: 19/137,621

(22) PCT Filed: Nov. 28, 2023
(86) PCT No.: PCT/JP2023/042549

§ 371 Date: Jun. 10, 2025

(30) Foreign Application Priority Data
Dec. 15, 2022 (JP) 2022-200359

(51) Int. Cl. G03F 7/004; C07C 69/757; C07C 235/82; C07C 237/26; C07C 381/12; C07D 333/76; G03F 7/00; G03F 7/038; G03F 7/039; G03F 7/20; H10P 76/20

(52) U.S. Cl. CPC G03F 7/0045; C07C 69/757; C07C 235/82; C07C 237/26; C07C 381/12; C07D 333/76; G03F 7/0382; G03F 7/0388; G03F 7/0397; C07C 2602/42; C07C 2603/74; C07C 2603/88; G03F 7/2002; G03F 7/70008; H10P 76/2041

(57) ABSTRACT

A resist composition including a base material component whose solubility in a developing solution is changed by an action of an acid and a compound represented by General Formula (d0). In Formula (d0), Rf represents a hydrocarbon group which may have a fluorine atom; L² represents a divalent cyclic group which may have a substituent; L¹ and L³ each independently represent a divalent linking group; x represents an integer of 1 to 4; R^{d0} represents an iodine atom or a bromine atom; y represents an integer of 1 to 4; z represents an integer of 0 to 3; 2≤x+y+z≤5 is satisfied; (M^{m+})_{1/m} represents a counter cation; and m represents an integer of 1 or greater. Both high sensitivity and roughness reduction can be achieved and a pattern having a satisfactory shape with high rectangularity can be formed when forming a resist pattern

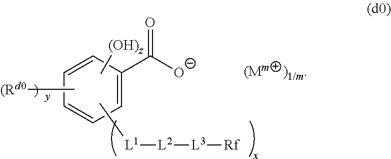

### TECHNICAL FIELD

The present invention relates to a resist composition, a resist pattern formation method, a compound, and an acid diffusion control agent.

Priority is claimed on Japanese Patent Application No. 2022-200359, filed Dec. 15, 2022, the content of which is incorporated herein by reference.

### BACKGROUND ART

In recent years, in the manufacture of semiconductor elements and liquid crystal display elements, advances in lithography technologies have led to a rapid progress in the field of pattern fining. These pattern fining techniques typically involve shortening the wavelength (increasing the energy) of the exposure light source.

Resist materials are required to have lithography characteristics such as sensitivity to these exposure light sources and resolution that enables reproduction of patterns with minute dimensions.

As a resist material that satisfies these requirements, a chemically amplified resist composition containing a base material component whose solubility in a developing solution is changed by an action of an acid and an acid generator component that generates an acid upon light exposure has been used in the related art.

In the resist pattern formation, the behavior of an acid generated from an acid generator component upon light exposure is considered as one factor that has a great influence on lithography characteristics.

As the acid generator that is used in the chemically amplified resist composition, a wide variety of acid generators have been suggested in the related art. Examples of such known acid generators include an onium salt-based acid generator such as an iodonium salt or a sulfonium salt, an oxime sulfonate-based acid generator, a diazomethane-based acid generator, a nitrobenzyl sulfonate-based acid generator, an imino sulfonate-based acid generator, and a disulfone-based acid generator.

An alkyl sulfonate ion or a fluorinated alkyl sulfonate ion in which some or all hydrogen atoms of the alkyl group have been substituted with fluorine atoms is typically used in an anion moiety of the onium salt-based acid generator. As a cation moiety of the onium salt-based acid generator, a cation moiety including an onium ion such as triphenylsulfonium is mainly used.

In addition, as the resist material, a chemically amplified resist composition containing a combination of an acid generator component and an acid diffusion control agent that controls diffusion of an acid to be generated from the acid generator component upon light exposure has been suggested in the related art.

For example, Patent Document 1 discloses a resist composition in which a carboxylate having a specific structure in an anion moiety is selected as an acid diffusion control agent.

### CITATION LIST

### Patent Document

Patent Document 1: Japanese Unexamined Patent Application, First Publication No. 2022-14782

### SUMMARY OF INVENTION

### Technical Problem

With further advances in lithography technologies, rapid progress in the field of pattern fining is being achieved together with the expansion and the like of application fields. In association with this, in a case of manufacturing a semiconductor element or the like, there is a demand for a technology that enables formation of a fine pattern in a satisfactory shape. For example, in lithography using extreme ultraviolet (EUV) rays or electron beams (EB), a target is to form a fine pattern with a size of several tens of nm. As the pattern dimensions decrease, there is a demand for further improvement of each of the lithography characteristics such as sensitivity, a pattern shape, and roughness reduction without trade-off.

The present invention has been made in consideration of the above-described circumstances, and an object thereof is to provide a resist composition capable of achieving both high sensitivity and roughness reduction in a case of forming a resist pattern and forming a pattern that has a satisfactory shape with high rectangularity, a resist pattern formation method using the resist composition, and a compound useful as a base component used in the resist composition.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition including: a base material component (A) whose solubility in a developing solution is changed by the action of the acid; and a compound (D0) represented by General Formula (d0).

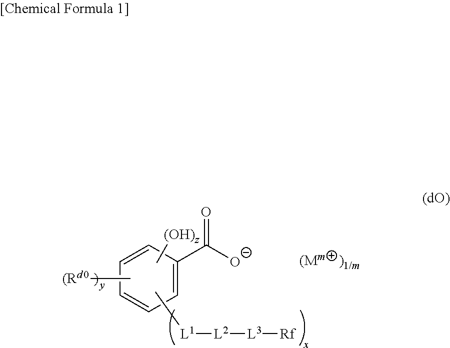

[In the formula, Rf represents a hydrocarbon group which may have a fluorine atom. L² represents a divalent cyclic group which may have a substituent. L¹ and L³ each independently represent a divalent linking group. x represents an integer of 1 to 4. In a case where x represents an integer of 2 or greater, a plurality of (-L¹-L²-L³-Rf)'s may be the same as or different from each other. R^{d0} represents an iodine atom or a bromine atom. y represents an integer of 1 to 4. In a case where y represents an integer of 2 or greater, a plurality of R^{d}0's may be the same as or different from each other. z represents an integer of 0 to 3. Here, 2≤x+y+z≤5 is satisfied. (M^{m+})_{1/m} represents a counter cation. m represents an integer of 1 or greater.]

According to a second aspect of the present invention, there is provided a resist pattern formation method including: a step of forming a resist film on a support using the resist composition according to the first aspect; a step of exposing the resist film to light; and a step of developing the resist film exposed to light to form a resist pattern.

According to a third aspect of the present invention, there is provided a compound represented by General Formula (d0).

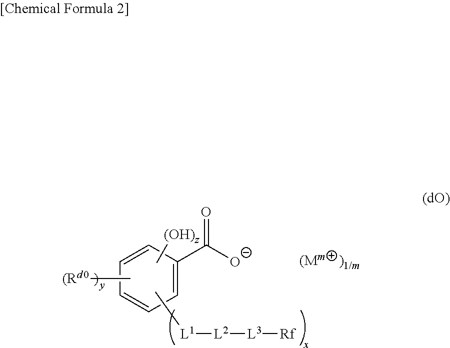

[In the formula, Rf represents a hydrocarbon group which may have a fluorine atom. L² represents a divalent cyclic group which may have a substituent. L¹ and L³ each independently represent a divalent linking group. x represents an integer of 1 to 4. In a case where x represents an integer of 2 or greater, a plurality of (-L¹-L²-L³-Rf)'s may be the same as or different from each other. R^{d0} represents an iodine atom or a bromine atom. y represents an integer of 1 to 4. In a case where y represents an integer of 2 or greater, a plurality of R^{d}0's may be the same as or different from each other. z represents an integer of 0 to 3. Here, 2≤x+y+z≤5 is satisfied. (M^{m+})_{1/m} represents a counter cation. m represents an integer of 1 or greater.]

According to a fourth aspect of the present invention, there is provided an acid diffusion control agent containing the compound according to the third aspect described above.

### Advantageous Effects of Invention

According to the present invention, it is possible to provide a resist composition capable of achieving both high sensitivity and roughness reduction in a case of forming a resist pattern and forming a pattern that has a satisfactory shape with high rectangularity, a resist pattern formation method using the resist composition, and a compound useful as a base component used in the resist composition.

### DESCRIPTION OF EMBODIMENTS

In the present specification and the scope of the present claims, the term “aliphatic” is a relative concept used with respect to “aromatic” and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of the entire base material component increases. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, the term “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the a-position may be substituted with a substituent. The substituent (R^{αx}) that substitutes the hydrogen atom bonded to the carbon atom at the a-position is an atom other than the hydrogen atom or a group. Further, itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes a hydrogen atom at the α-position of hydroxystyrene include the same groups as those for R^{αx}, such as an alkyl group and a halogenated alkyl group.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the first aspect of the present invention is a resist composition that generates an acid upon light exposure and whose solubility in a developing solution is changed by the action of the acid.

According to one embodiment, such a resist composition contains a base material component (A) (hereinafter, also referred to as “component (A)”) whose solubility in a developing solution is changed by the action of the acid, and a base component (hereinafter, also referred to as “component (D)”) that traps an acid generated upon light exposure (that is, controls the diffusion of the acid). In the resist composition according to the present embodiment, a compound (D0) represented by General Formula (d0) is used as the component (D).

In the resist composition according to the present embodiment, the component (A) may generate an acid upon light exposure, or a blended component that is blended separately from the component (A) may generate an acid upon light exposure. Specifically, the resist composition according to the present embodiment may (1) further contain an acid generator component (B) (hereinafter, referred to as “component (B)”) that generates an acid upon light exposure; (2) have a component (A) that generates an acid upon light exposure; and (3) have a component (A) that generates an acid upon light exposure and further contains component (B).

That is, in the cases of (2) and (3) described above, the component (A) is “base material component which generates an acid upon light exposure and whose solubility in a developing solution is changed by the action of the acid”. In a case where the component (A) is a base material component which generates an acid upon light exposure and whose solubility in a developing solution is changed by the action of the acid, it is preferable that the component (A1) described below is a resin which generates an acid upon light exposure and whose solubility in a developing solution is changed by the action of the acid. As such a resin, a polymer compound having a constitutional unit that generates an acid upon light exposure can be used. As the constitutional unit that generates an acid upon light exposure, those which have been known can be used.

Among the examples, it is preferable that the resist composition according to the present embodiment corresponds to the case (1). That is, it is preferable that the resist composition according to the present embodiment contains the component (A), the compound (D0), and the component (B).

In a case where a resist film is formed using the resist composition according to the present embodiment and the formed resist film is subjected to selective exposure, for example, an acid is generated from the component (B) at exposed portions of the resist film, and the solubility of the component (A) in a developing solution is changed by the action of the acid, whereas the solubility of the component (A) in a developing solution is not changed at unexposed portions of the resist film, and thus a difference in solubility in the developing solution occurs between the exposed portions and the unexposed portions of the resist film. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

The resist composition of the present embodiment may be a positive-tone resist composition or a negative-tone resist composition. Further, in the formation of a resist pattern, the resist composition according to the present embodiment may be applied to an alkali developing process using an alkali developing solution in the developing treatment, or a solvent developing process using a developing solution containing an organic solvent (organic developing solution) in the developing treatment.

### <Component (A): Base Material Component>

In the resist composition according to the present embodiment, it is preferable that the component (A) has a resin component (A1) whose solubility in a developing solution is changed by the action of an acid (hereinafter, also referred to as “component (A1)”). Since the polarity of the base material component before and after the light exposure is changed by using the component (A1), a satisfactory development contrast can be obtained not only in an alkali developing process but also in a solvent developing process.

As the component (A), at least one of other polymer compounds or low-molecular-weight compounds may be used in combination with the component (A1).

In the resist composition according to the present embodiment, the component (A) may be used alone or in combination of two or more kinds thereof.

### In Regard to Component (A1)

The component (A1) is a resin component whose solubility in a developing solution is changed by the action of an acid.

As the component (A1), those having a constitutional unit (a1) containing an acid decomposable group whose polarity is increased by the action of an acid are preferable. Further, the component (A1) may have other constitutional units as necessary in addition to the constitutional unit (a1).

Examples of the other constitutional units include a constitutional unit (a10) represented by General Formula (a10-1), a constitutional unit (a2) containing a lactone-containing cyclic group, and a constitutional unit (a8) derived from a compound represented by General Formula (a8-1).

### In Regard to Constitutional Unit (a1):

The constitutional unit (a1) is a constitutional unit that contains an acid decomposable group whose polarity is increased due to the action of an acid.

Examples of the acid decomposable group include a group in which a polar group is protected by an acid dissociable group (for example, a group in which a hydrogen atom of an OH-containing polar group is protected by an acid dissociable group).

Examples of the acid dissociable group are the same as those which have been suggested as the acid dissociable groups of the base resin for a chemically amplified resist composition.

Specific examples of the acid dissociable group of the base resin suggested for a chemically amplified resist composition include “acetal type acid dissociable group”, “tertiary alkyl ester type acid dissociable group”, “tertiary alkyloxycarbonyl acid dissociable group”, and “secondary alkyl ester type acid dissociable group” described below.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

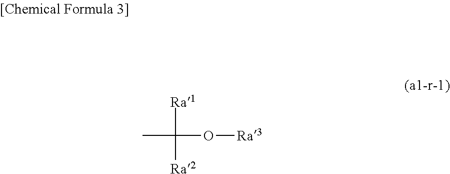

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom. In a case where Ra′¹ or Ra′² represents an alkyl group, examples of the alkyl group include the same alkyl groups described as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may have a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of one kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2). Among examples of the acid dissociable group represented by Formula (a1-r-2), hereinafter, a group formed of an alkyl group will also be referred to as “tertiary alkyl ester type acid dissociable group” for convenience.

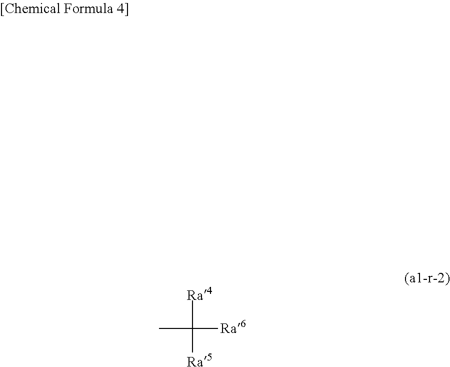

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, a chain-like alkynyl group, and a cyclic hydrocarbon group.

Examples of the linear or branched alkyl group and the cyclic hydrocarbon group (an alicyclic hydrocarbon group which is a monocyclic group, an alicyclic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to each other, suitable examples thereof include a group represented by General Formula (a1-r2-4).

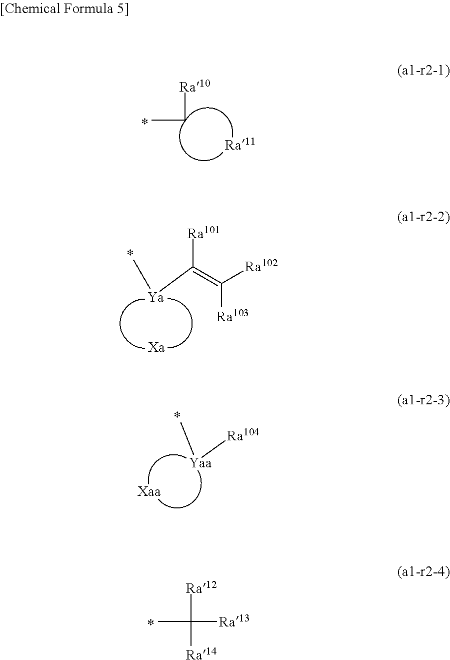

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra′¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a chain-like monovalent saturated hydrocarbon group having 1 to 10 carbon atoms. Some or all hydrogen atoms contained in the chain-like saturated hydrocarbon group may be substituted. Ra′14 represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include the same groups as those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the alicyclic hydrocarbon group (alicyclic hydrocarbon group) which is a monocyclic group or a polycyclic group as Ra′³ in Formula (a1-r-1). Among them, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (alicyclic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same groups as those for Ra^{x5}.

Examples of the group having a carbon-carbon double bond generated by two or more of Ra⁰¹ to Raio³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group described as the alicyclic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a chain-like monovalent saturated hydrocarbon group having 1 to 10 carbon atoms. Examples of the chain-like monovalent saturated hydrocarbon group having 1 to 10 carbon atoms as Ra′¹² and Ra′¹³ include the same one as the chain-like monovalent saturated hydrocarbon group having 1 to 10 carbon atoms as Ra¹⁰¹ to Ra¹⁰³ as described above. Some or all hydrogen atoms contained in the chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent include the same groups as those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an alicyclic hydrocarbon group or an aromatic hydrocarbon group and may be a polycyclic group or a monocyclic group.

The alicyclic hydrocarbon group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be any of the 1-position or the 2-position of the naphthyl group.

In a case where Ra′¹⁴ in Formula (a1-r2-4) represents an anthryl group, the position bonded to the tertiary carbon atom in Formula (a1-r2-4) may be any of the 1-position, the 2-position, or the 9-position of the anthryl group.

Specific examples of the group represented by Formula (a1-r2-1) are shown below.

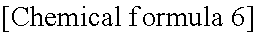

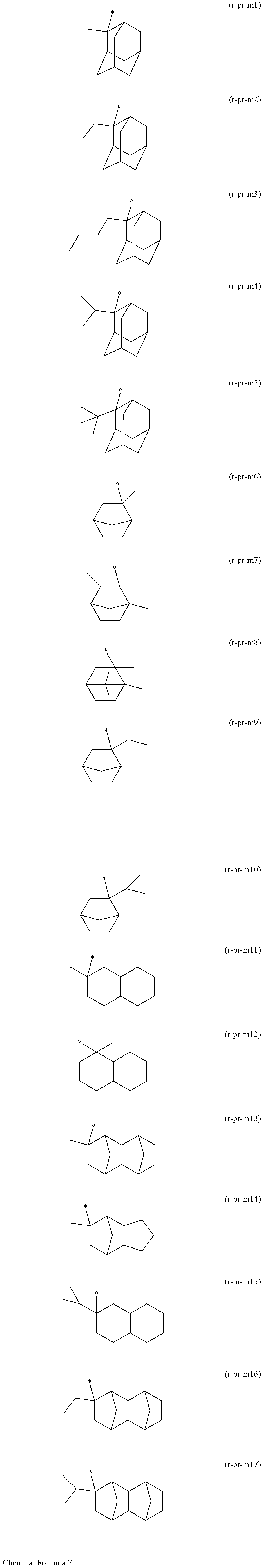

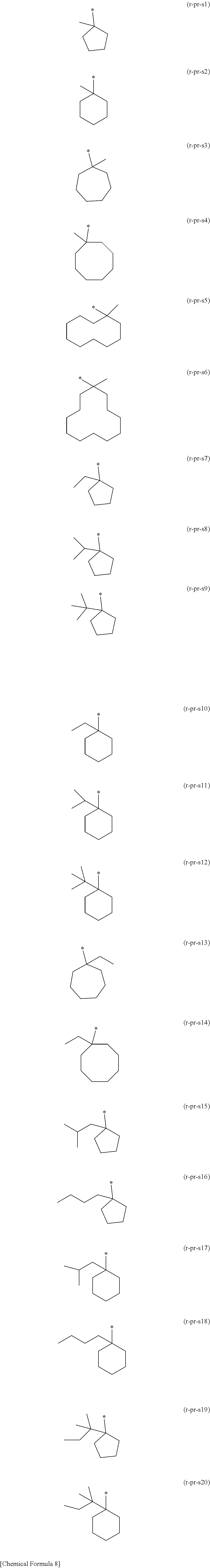

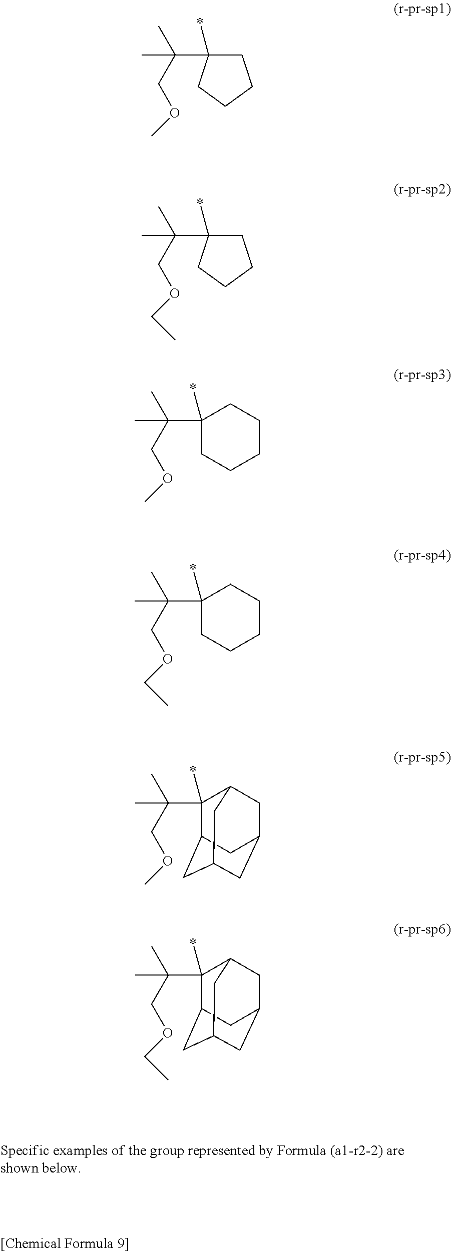 [SMILES: *C1(C(C)(C)COC)CCCC1 ; *C1(C(C)(C)COCC)CCCC1 ; *C1(C(C)(C)COC)CCCCC1 ; *C1(C(C)(C)COCC)CCCCC1 ; *C1(C(C)(C)COC)C2CC3CC(C2)CC1C3 ; *C1(C(C)(C)COCC)C2CC3CC(C2)CC1C3]

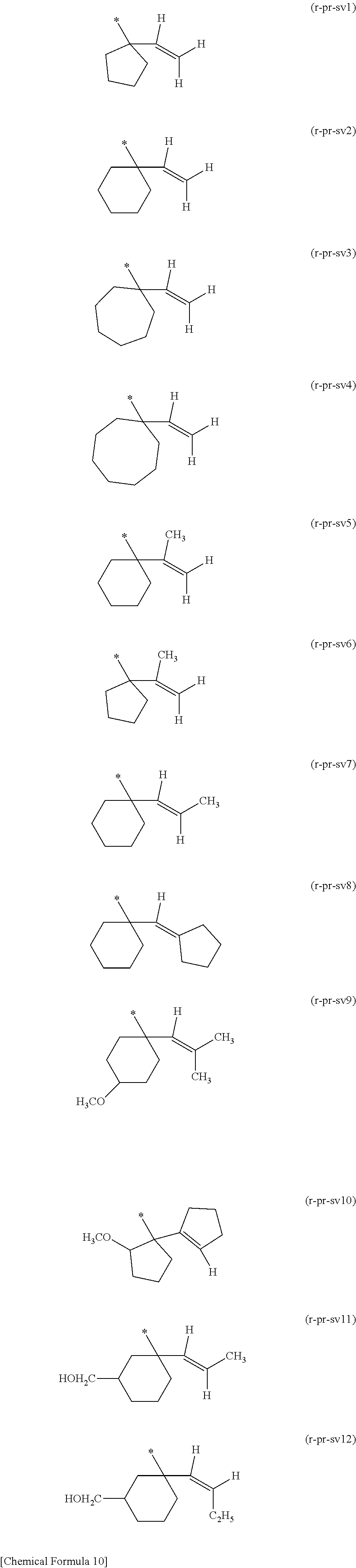

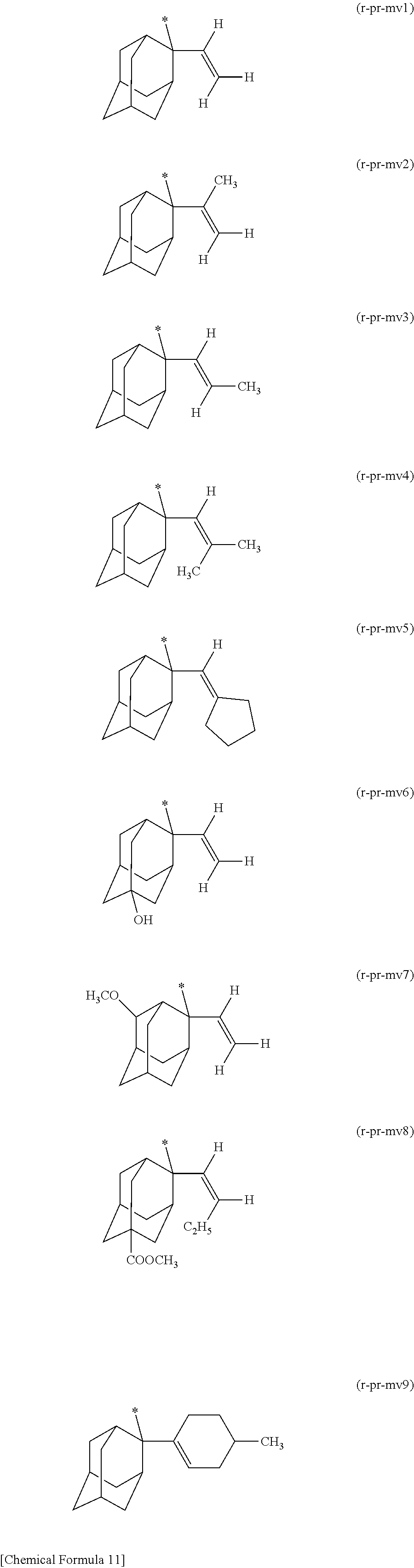

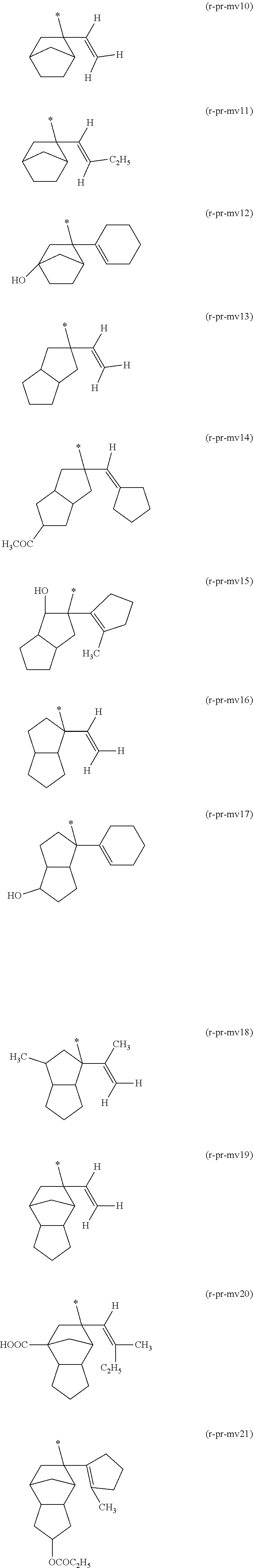

Specific examples of the group represented by Formula (a1-r2-3) are shown below.

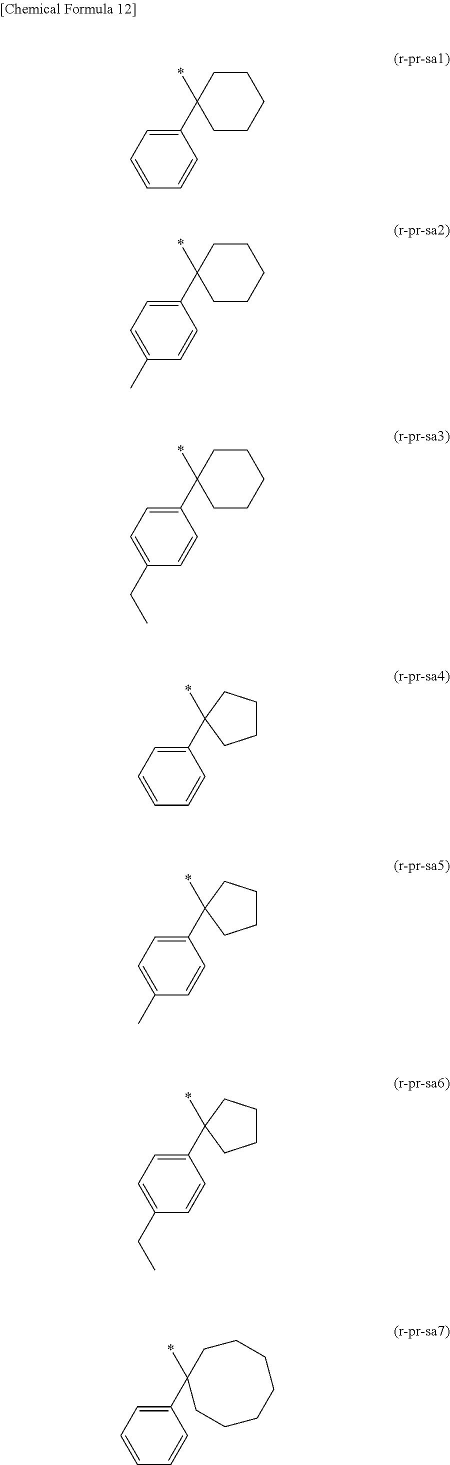

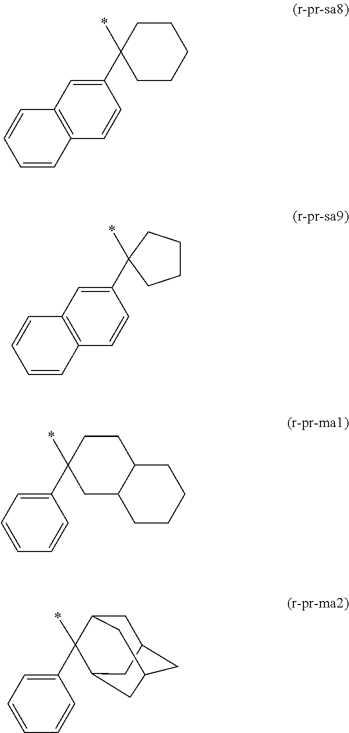 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

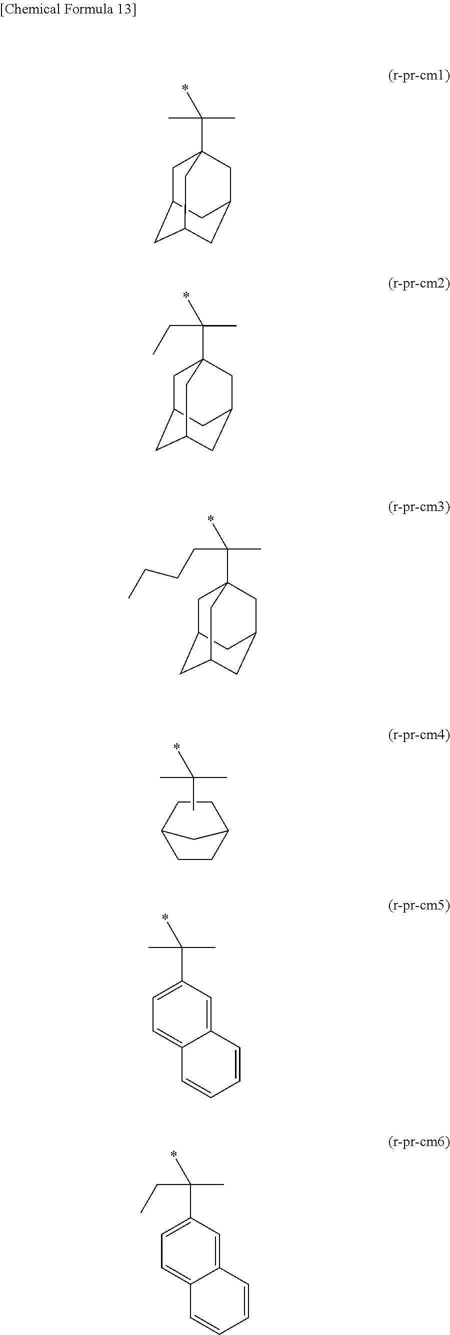

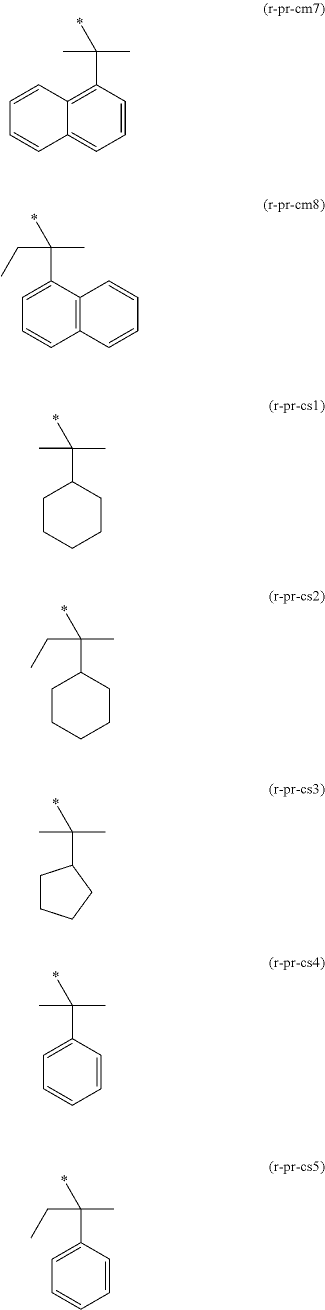

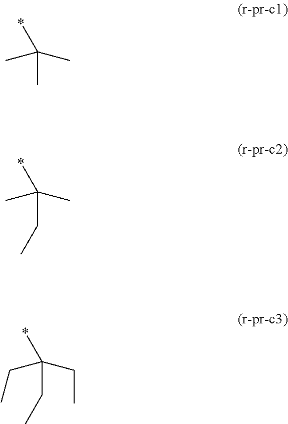

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

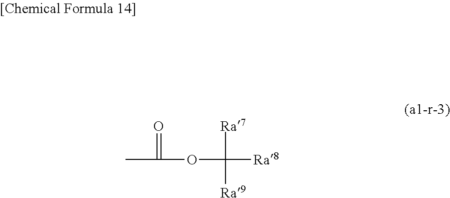

[In the formula, Ra′⁷ to Ra′⁹ each represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

### Secondary Alkyl Ester Type Acid Dissociable Group:

Examples of the acid dissociable group that protects a carboxy group among the polar groups include an acid dissociable group represented by General Formula (a1-r-4).

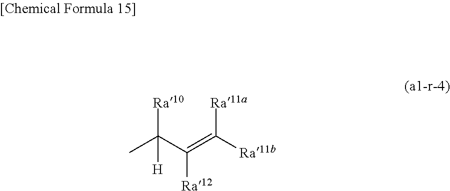

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of this substituent include Ra^{x5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the same rings as those formed by Ra′¹⁰ and Ra′^{11a} or Ra′^{11b} being bonded to each other.

Specific examples of the group represented by Formula (a1-r-4) are shown below.

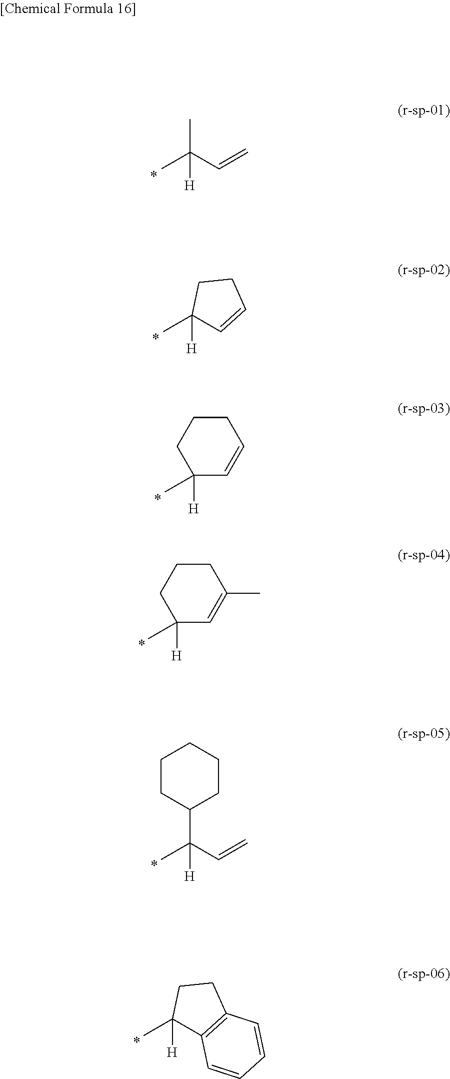

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include a constitutional unit represented by General Formula (a1-1), (a1-2), or (a1-3).

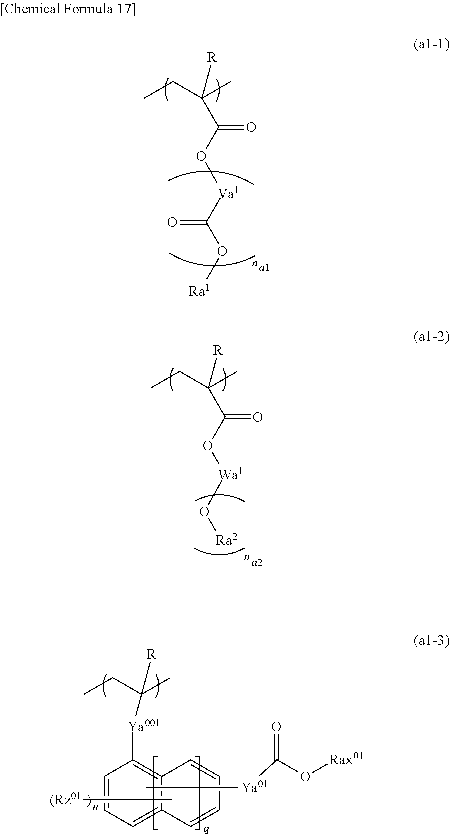

[In the formula, R represents a hydrogen atom, an alkyl group having 1 to 5 carbon atoms, or a halogenated alkyl group having 1 to 5 carbon atoms. Va¹ represents a divalent hydrocarbon group which may contain an ether bond. nₐ₁ represents an integer of 0 to 2. Ra¹ represents an acid dissociable group represented by General Formula (a1-r-1), (a1-r-2), or (a1-r-4). Wa¹ represents an (nₐ₂+1)-valent hydrocarbon group. nₐ₂ represents an integer of 1 to 3. Ra² represents an acid dissociable group represented by General Formula (a1-r-1) or (a1-r-3). Ya⁰⁰¹ represents a single bond or a divalent linking group. Ya⁰¹ represents a single bond or a divalent linking group. Rax⁰¹ represents an acid dissociable group represented by General Formula (a1-r-1), (a1-r-2), or (a1-r-4). Rz⁰¹ represents an alkyl group, a halogen atom, a halogenated alkyl group, a hydroxy group, or an alkoxy group. q represents an integer of 0 to 3. n represents an integer of 0 or greater. Here, n≤q×2+4 is satisfied.]

In Formulae (a1-1) to (a1-3), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents preferably an acid dissociable group represented by General Formula (a1-r-2) or (a1-r-4) and, among these, more preferably a group represented by General Formula (a1-r2-1) or an acid dissociable group represented by General Formula (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent and more preferably divalent or trivalent.

In Formula (a1-2), it is preferable that Ra² represents an acid dissociable group represented by General Formula (a1-r-1).

In Formula (a1-3), the divalent linking group as Ya⁰⁰¹ is not particularly limited, and suitable examples thereof include a divalent hydrocarbon group which may have a substituent and a divalent linking group having a heteroatom.

Among these, it is preferable that Ya⁰¹ represents an ester bond [—C(═O)—O— or —O—C(═O)—], an ether bond (—O—), a linear or branched alkylene group, an aromatic hydrocarbon group, a combination thereof, or a single bond. The alkylene group has preferably 1 to 10 carbon atoms, more preferably 1 to 6 carbon atoms, still more preferably 1 to 4 carbon atoms, and particularly preferably 1 to 3 carbon atoms.

Among these, Ya⁰⁰¹ represents more preferably a combination of an ester bond [—C(═O)—O— or —O—C(═O)—] and a linear alkylene group, or a single bond and still more preferably a single bond.

In Formula (a1-3), the divalent linking group as Ya⁰¹ is not particularly limited, and suitable examples thereof include a divalent hydrocarbon group which may have a substituent and a divalent linking group having a heteroatom.

Among these, it is preferable that Ya⁰¹ represents an ester bond [—C(═O)—O— or —O—C(═O)—], an ether bond (—O—), a linear or branched alkylene group, an aromatic hydrocarbon group, a combination thereof, or a single bond. Among these, Ya⁰¹ is more preferably a combination of an ester bond [—C(═O)—O— or —O—C(═O)—] and a linear alkylene group, or a single bond, and still more preferably a single bond.

In Formula (a1-3), Rax⁰¹ represents preferably an acid dissociable group represented by General Formula (a1-r-2) or (a1-r-4) and, among these, more preferably an acid dissociable group represented by General Formula (a1-r-2) and still more preferably a group represented by General Formula (a1-r2-1).

In Formula (a1-3), the alkyl group, the halogenated alkyl group, and the alkoxy group as Rz⁰¹ have preferably 1 to 10 carbon atoms, more preferably 1 to 5 carbon atoms, still more preferably 1 to 3 carbon atoms, and particularly preferably 1 or 2 carbon atoms. The alkyl group, the halogenated alkyl group, and the alkoxy group may be linear or branched.

As the halogen atom as Rz⁰¹, an iodine atom or a bromine atom is preferable. As the halogen atom of the halogenated alkyl group represented by Rz⁰¹, a fluorine atom, an iodine atom, or a bromine atom is preferable, and a fluorine atom is more preferable. Rz⁰¹ represents preferably an alkoxy group or a hydroxy group and more preferably a hydroxy group.

In Formula (a1-3), q represents an integer of 0 to 3. A benzene structure is formed in a case where q represents 0, a naphthalene structure is formed in a case where q represents 1, an anthracene structure is formed in a case where q represents 2, and a tetracene structure is formed in a case where q represents 3.

In Formula (a1-3), n represents an integer of 0 or greater, preferably an integer of 0 to 5, more preferably an integer of 0 to 3, and still more preferably 1 or 2. In a case where n represents an integer of 2 or greater, two or more Rz⁰¹'s may be the same as or different from each other.

In Formula (a1-3), n≤q×2+4 is satisfied. For example, in a case where q represents 1 and a naphthalene structure is formed, all six hydrogen atoms of the naphthalene may be substituted with Rz⁰¹'s. In addition, in the naphthalene, the substitution positions of Ya⁰⁰¹, a -Ya⁰¹-C(═O)—O—Ra⁰¹ group, and Rz⁰¹ are not particularly limited.

Specific examples of the constitutional unit (a1) are shown below.

In the formulae shown below, Ra represents a hydrogen atom, a methyl group, or a trifluoromethyl group.

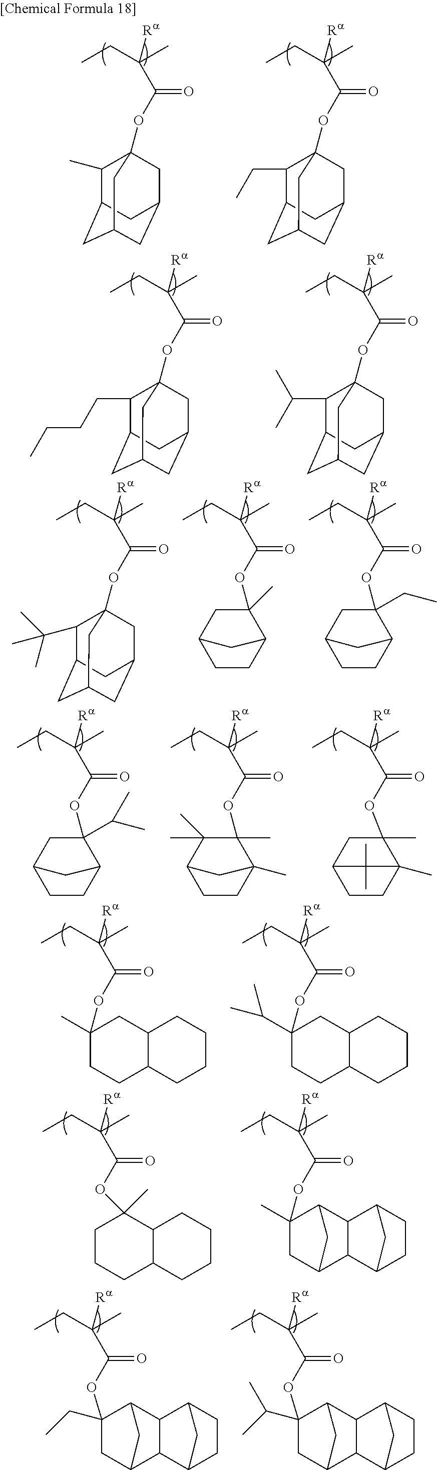

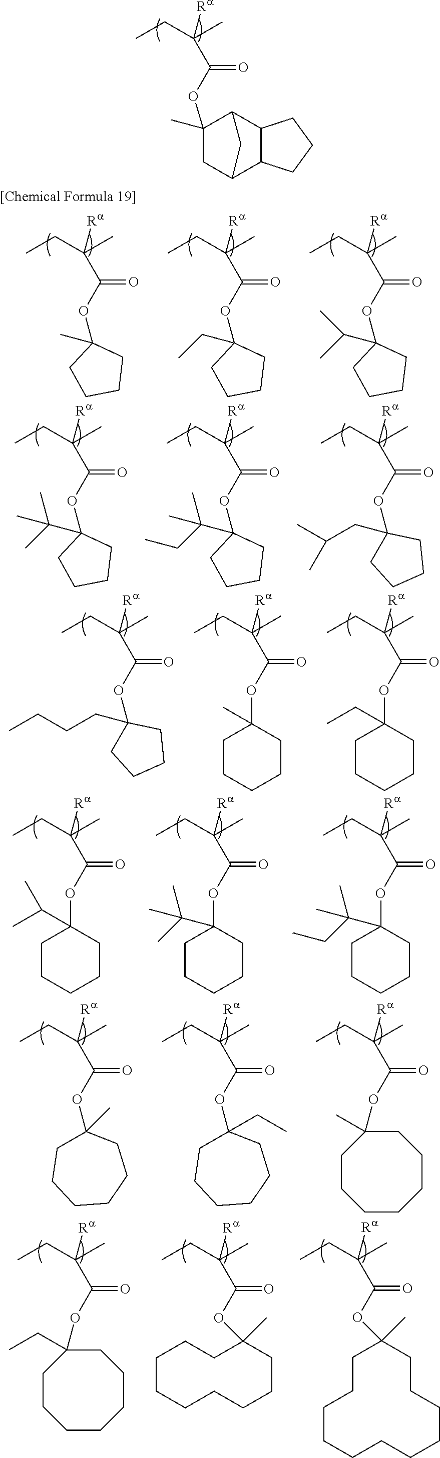

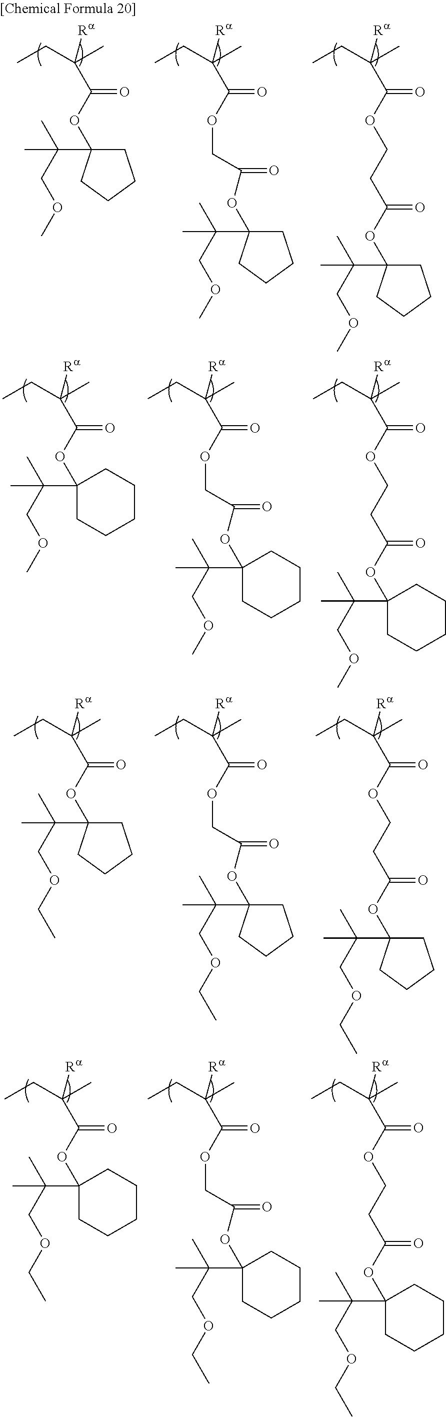

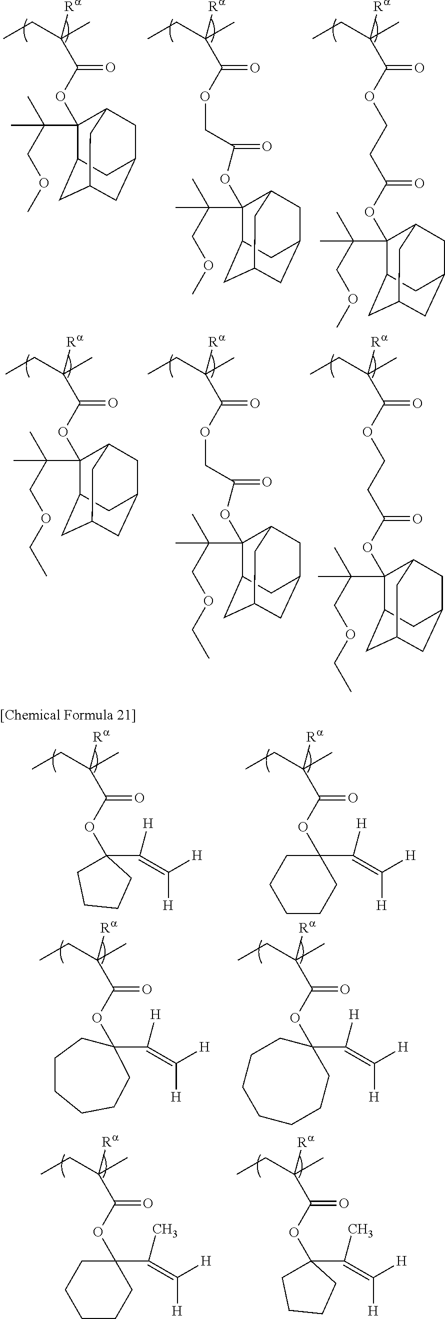

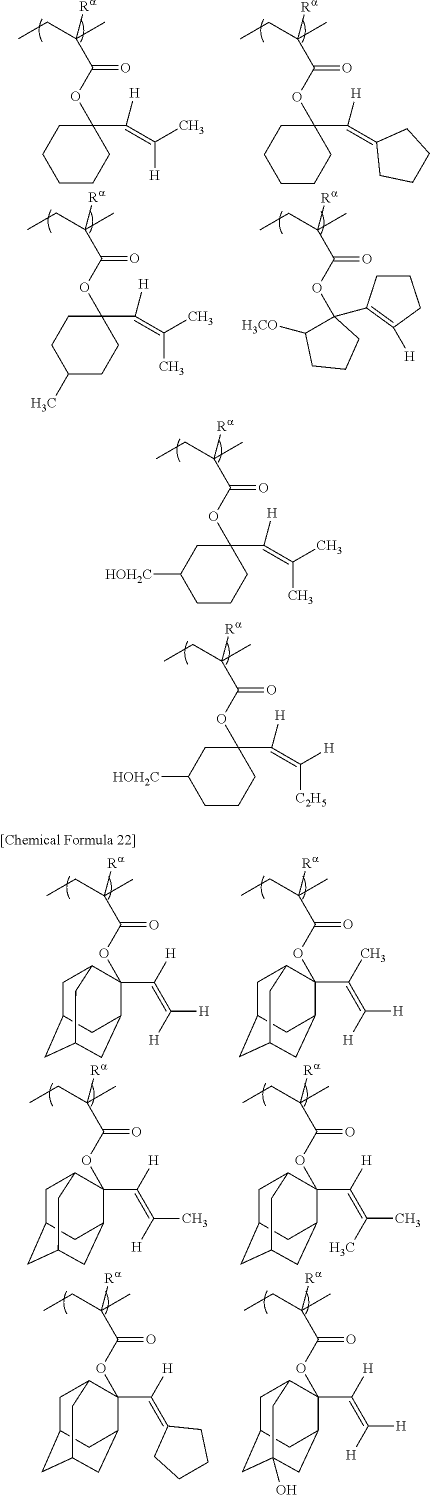

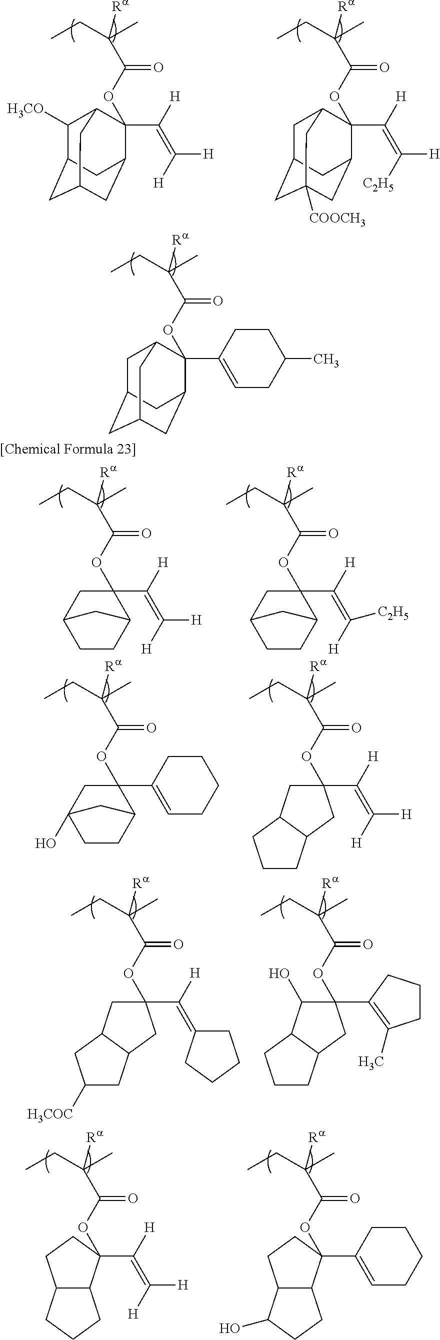

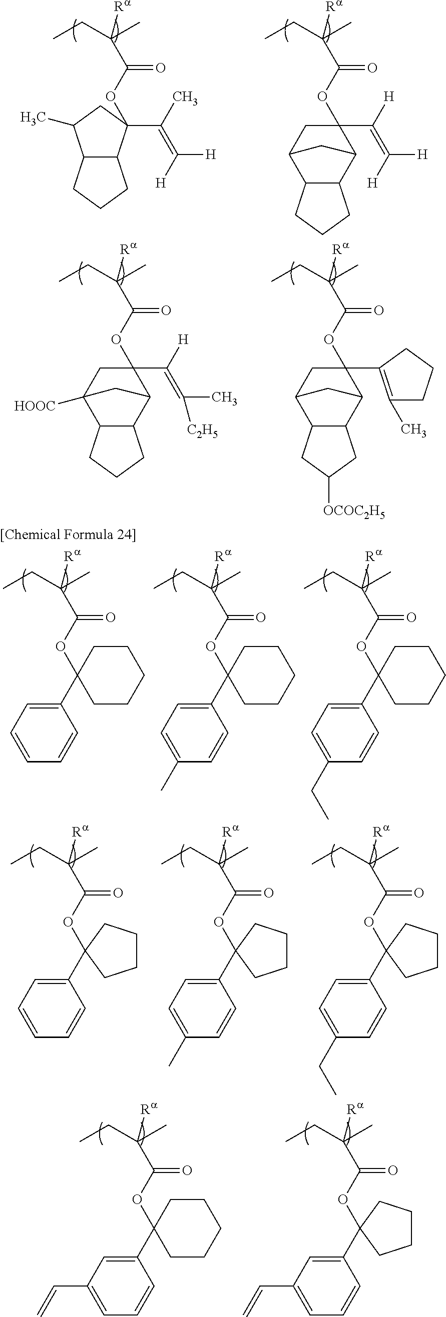

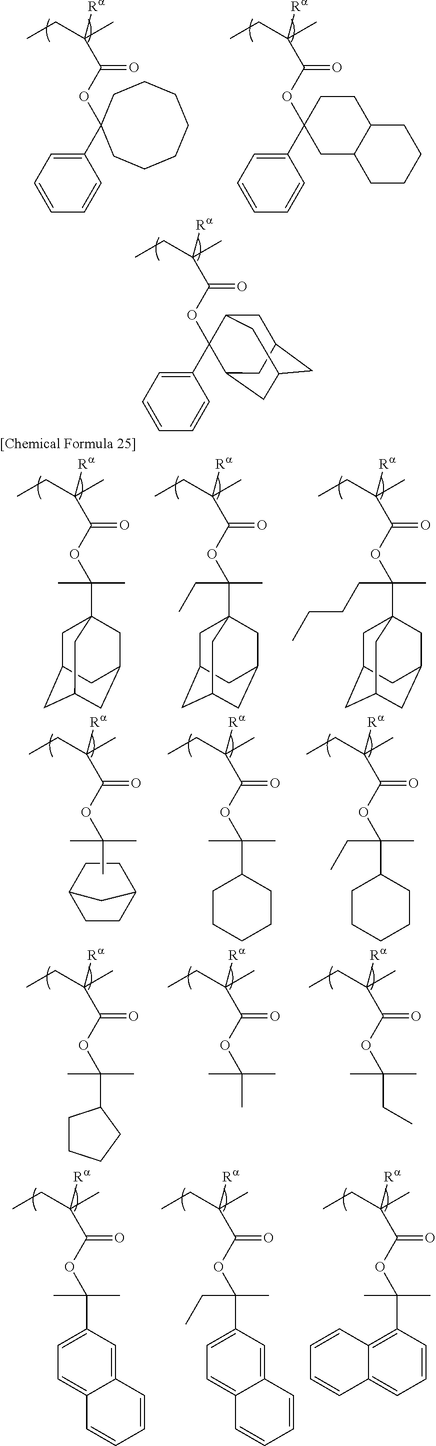

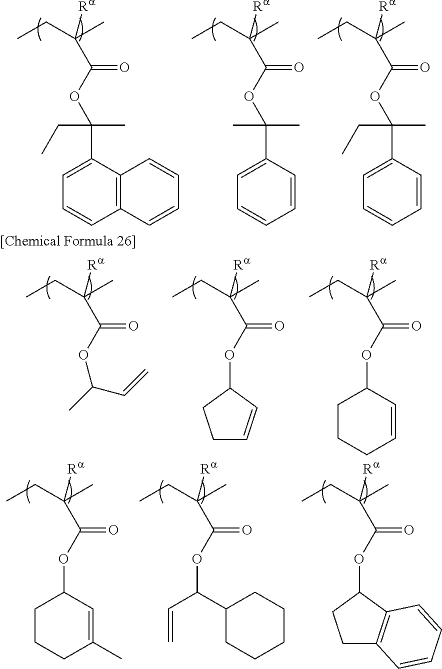

In the formulae shown below, R^{α} represents a hydrogen atom, a methyl group, or a trifluoromethyl group. Rz's each independently represent a hydrogen atom, an alkyl group, a halogen atom, a halogenated alkyl group, a hydroxy group, or an alkoxy group.

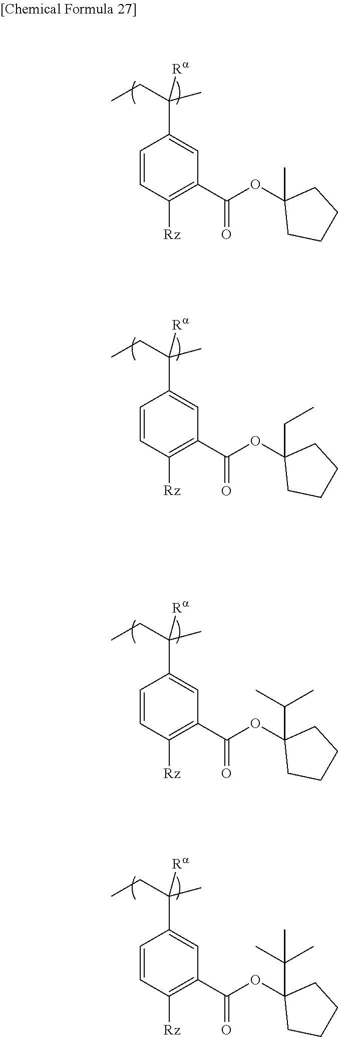

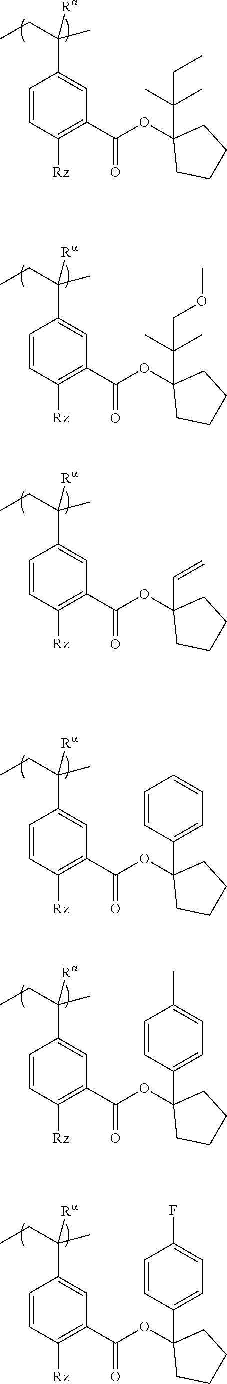

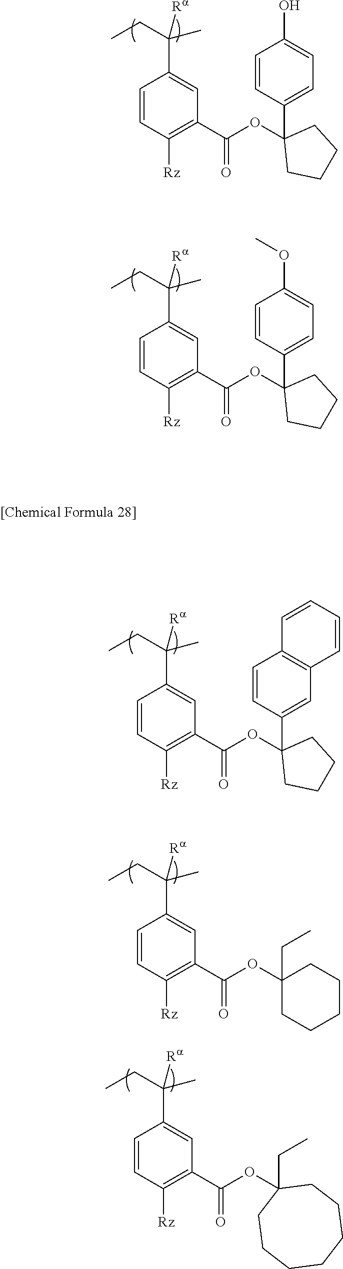

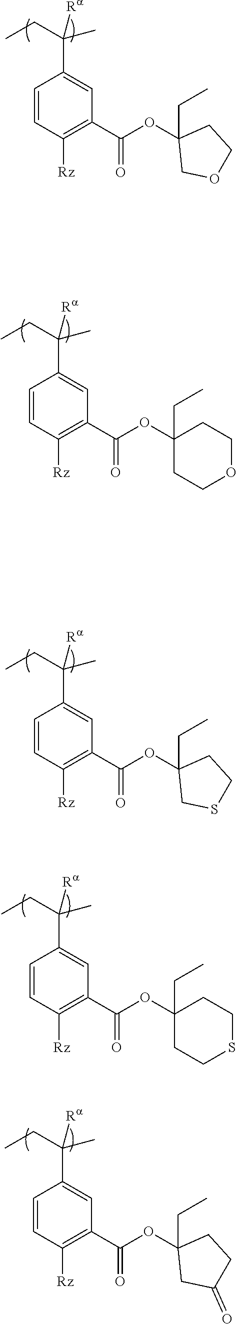

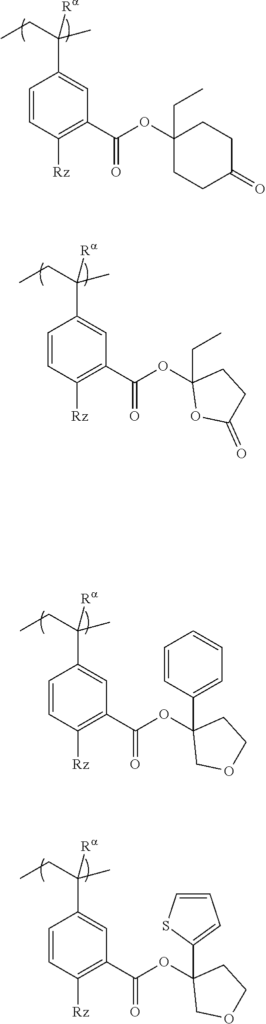

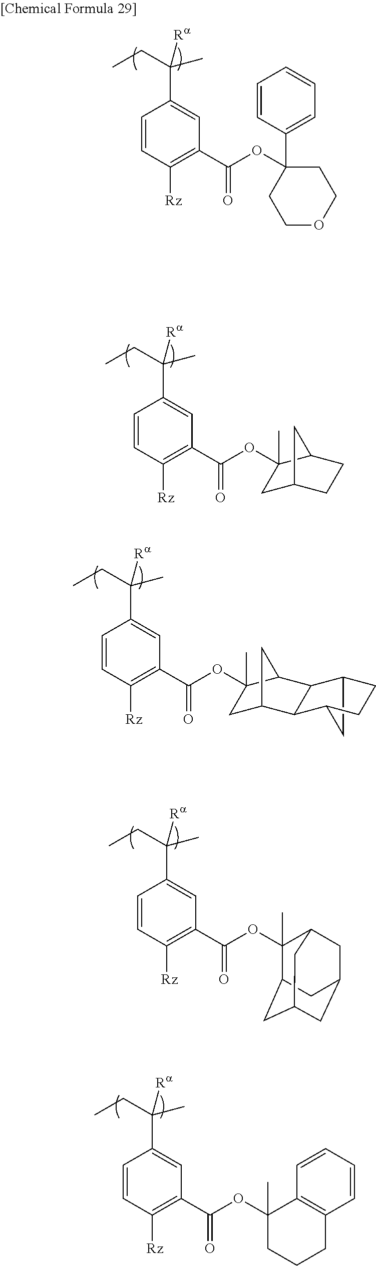

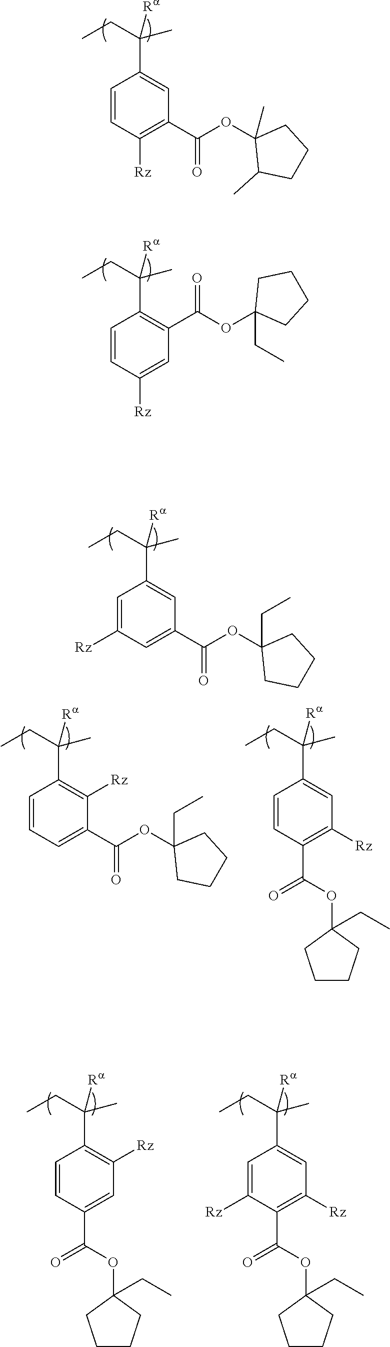

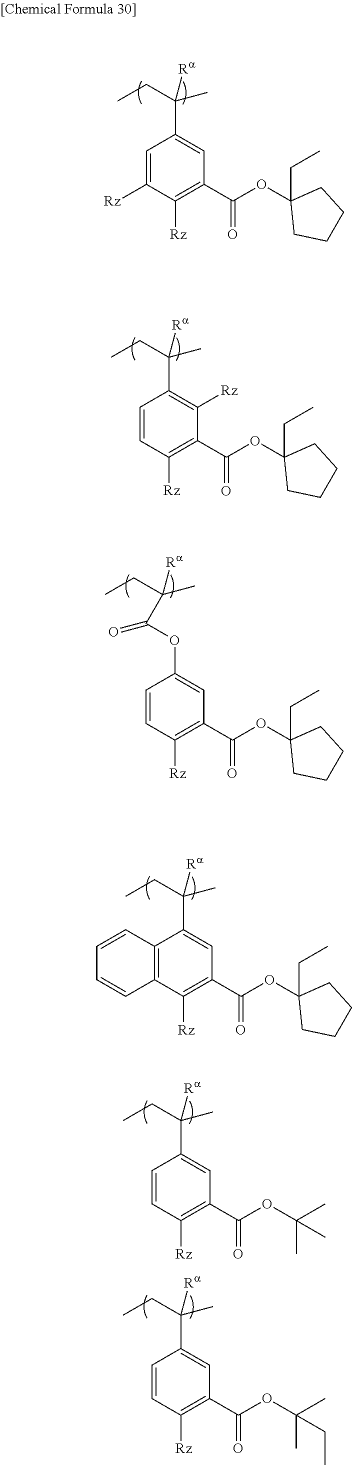

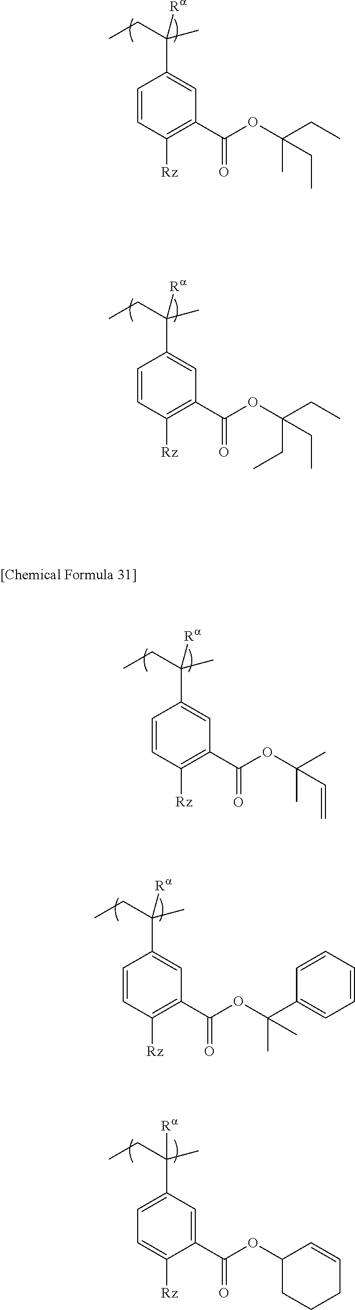

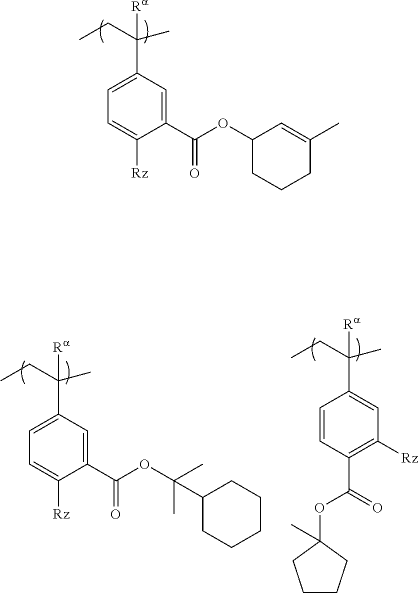

The constitutional unit (a1) included in the component (A1) may be used alone or two or more kinds thereof.

From the viewpoint of further easily enhancing the lithography characteristics (the sensitivity, the shape, and the like) using electron beams or EUV, a constitutional unit represented by Formula (a1-1) or a constitutional unit represented by Formula (a1-3) is more preferable as the constitutional unit (a1).

Among these, from the viewpoint of suitably increasing the reactivity for EB or EUV, the acid dissociable groups (Ra¹, Rax⁰¹) are each preferably an acid dissociable group represented by General Formula (a1-r2-1), (a1-r2-3), (a1-r2-4), or (a1-r-4). Among these, it is particularly preferable that the acid dissociable groups are selected from the cyclic groups.

In a case where the component (A1) has the constitutional unit (a1), the proportion of the constitutional unit (a1) in the component (A1) is preferably in a range of 20% to 80% by mole, more preferably in a range of 25% to 75% by mole, still more preferably in a range of 30% to 70% by mole, and particularly preferably in a range of 40% to 60% by mole with respect to the total amount (100% by mole) of all constitutional units constituting the component (A1).

In a case where the proportion of the constitutional unit (a1) is set to be greater than or equal to the lower values of the above-described preferable ranges, lithography characteristics such as sensitivity, rectangularity of a pattern shape, and roughness reduction are improved. Further, in a case where the proportion of the constitutional unit (a1) is less than or equal to the upper limits of the above-described preferable ranges, the constitutional unit (a1) and other constitutional units can be balanced, and various lithography characteristics are enhanced.

In Regard to Constitutional Unit (a10):

The constitutional unit (a10) is a constitutional unit represented by General Formula (a10-1) (here, a constitutional unit corresponding to the constitutional unit (a1) is excluded).

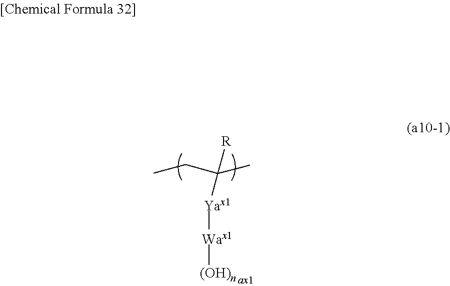

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In the Formula (a10-1), R represents a hydrogen atom, an alkyl group having 1 to 5 carbon atoms, or a halogenated alkyl group having 1 to 5 carbon atoms.

R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group. In the chemical formula, the divalent linking group as Ya^{x1} is not particularly limited, and suitable examples thereof include a divalent hydrocarbon group which may have a substituent and a divalent linking group having a heteroatom.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group obtained by removing (naₓ₁+1) hydrogen atoms from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (naₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include the same groups as those for the substituent of the cyclic alicyclic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

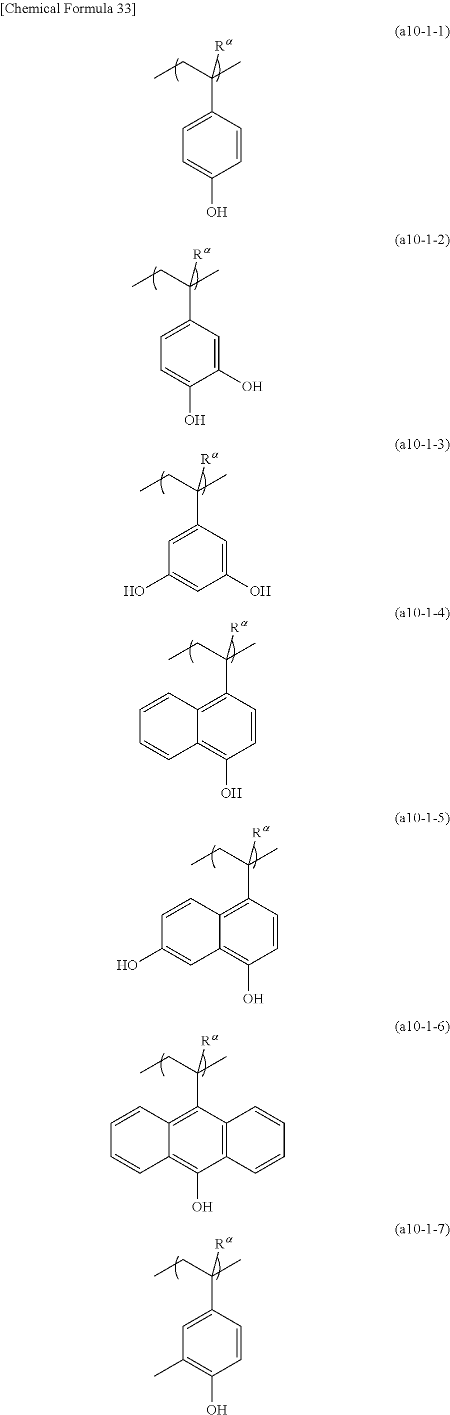

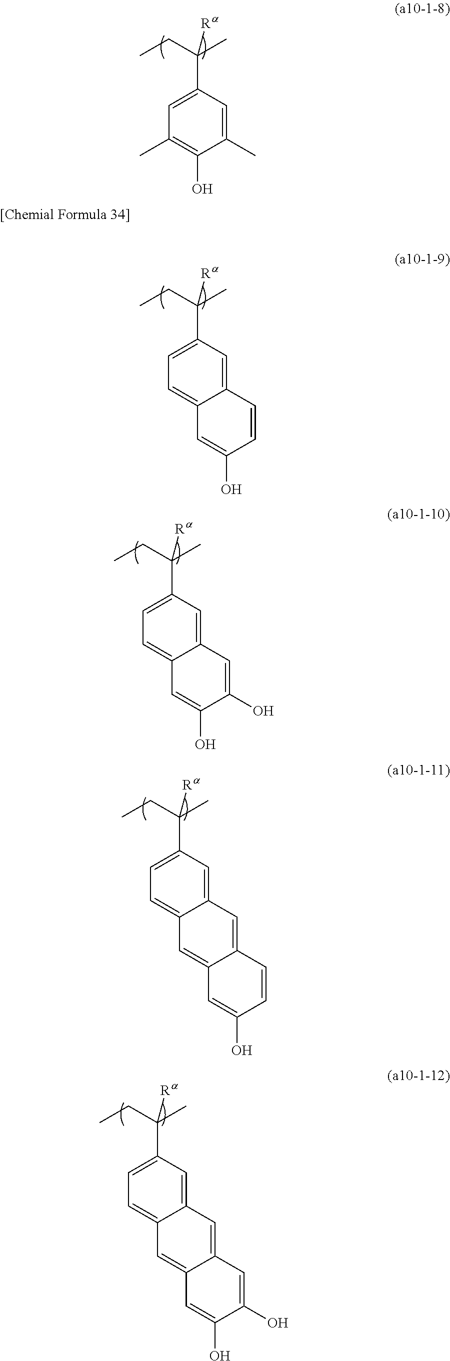

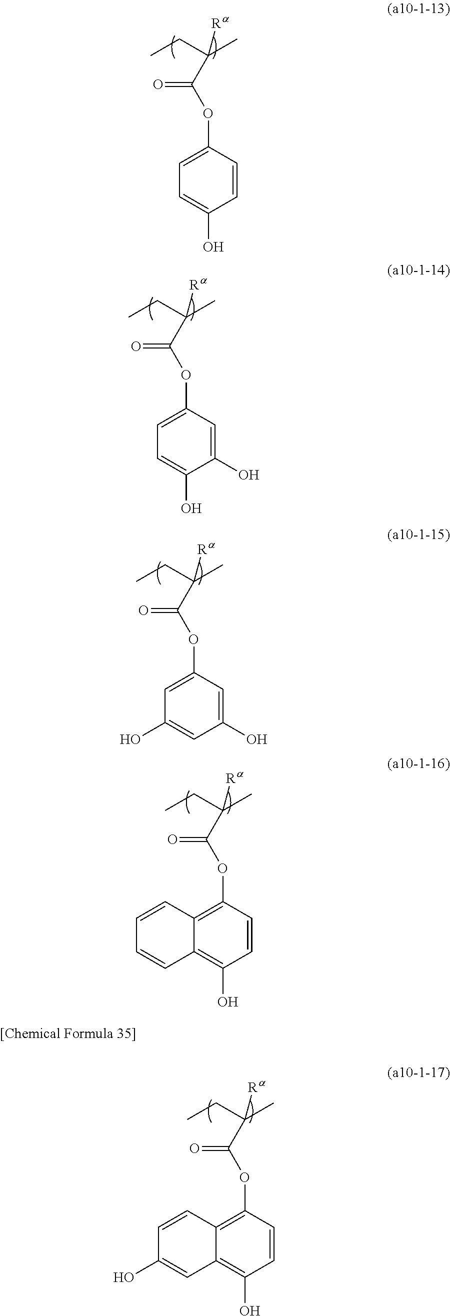

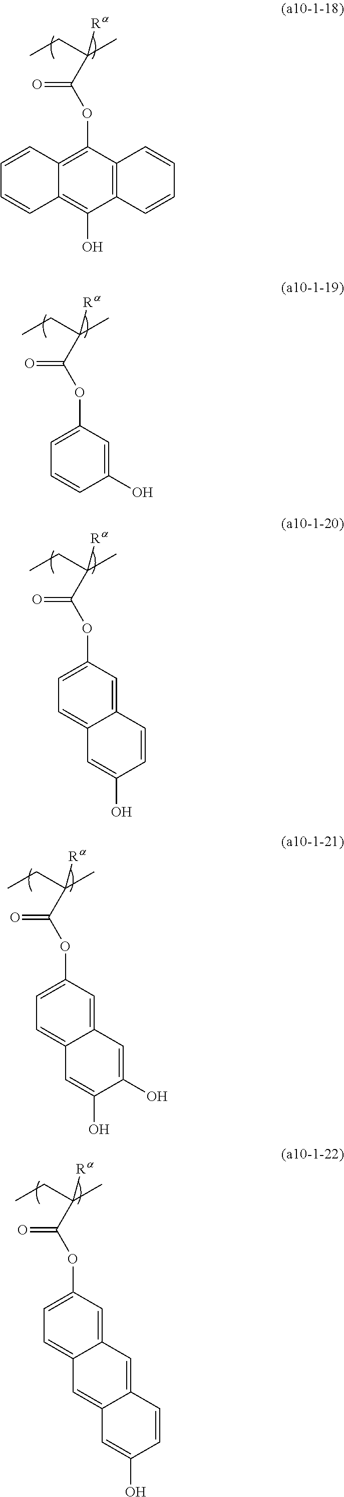

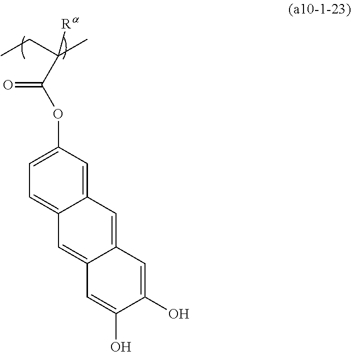

The constitutional unit (a10) included in the component (A1) may be used alone or two or more kinds thereof.

In a case where the component (A1) has the constitutional unit (a10), the proportion of the constitutional unit (a10) in the component (A1) is preferably in a range of 20% to 80% by mole, more preferably in a range of 25% to 75% by mole, still more preferably in a range of 30% to 70% by mole, and particularly preferably in a range of 40% to 60% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the upper limits, the constitutional unit (a10) and other constitutional units are likely to be balanced.

### In Regard to Constitutional Unit (a2):

The component (A1) may further have a constitutional unit (a2) (here, a constitutional unit corresponding to the constitutional unit (a1) is excluded) containing a lactone-containing cyclic group.

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are enhanced, for example, due to the effects of appropriately adjusting the acid diffusion length, increasing the adhesiveness of the resist film to the substrate, appropriately adjusting the solubility during the development, and the like.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

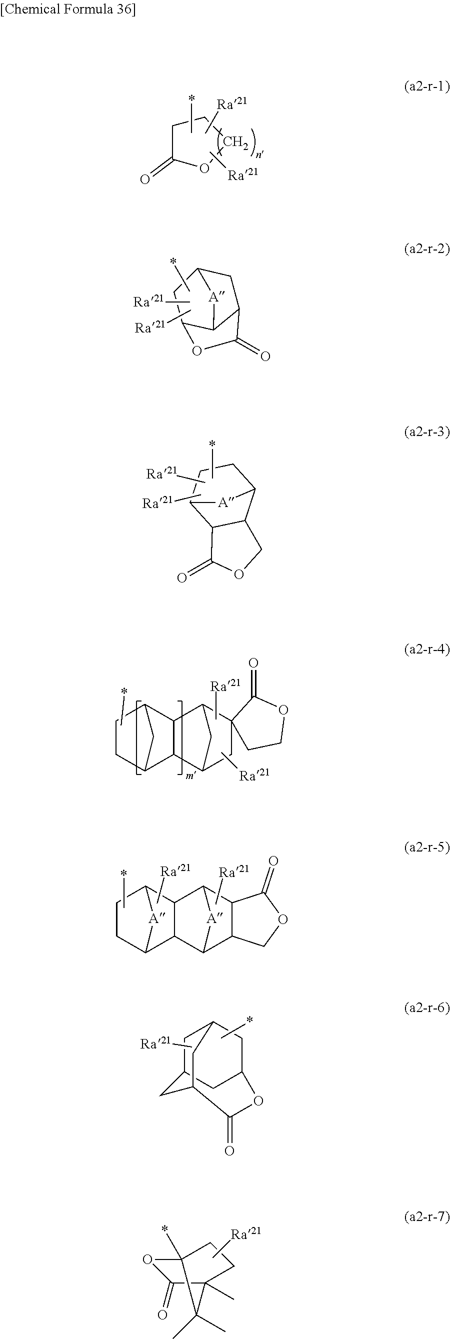

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), it is preferable that the alkyl group as Ra′²¹ is an alkyl group having 1 to 6 carbon atoms. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that each Ra′²¹ independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, and examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

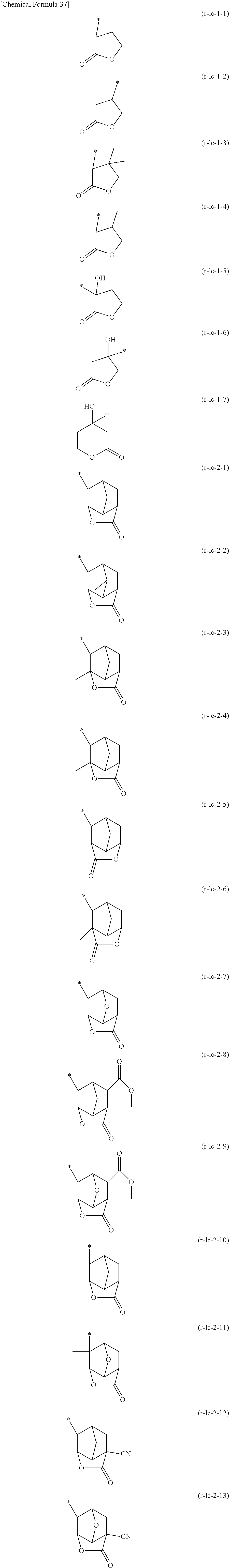

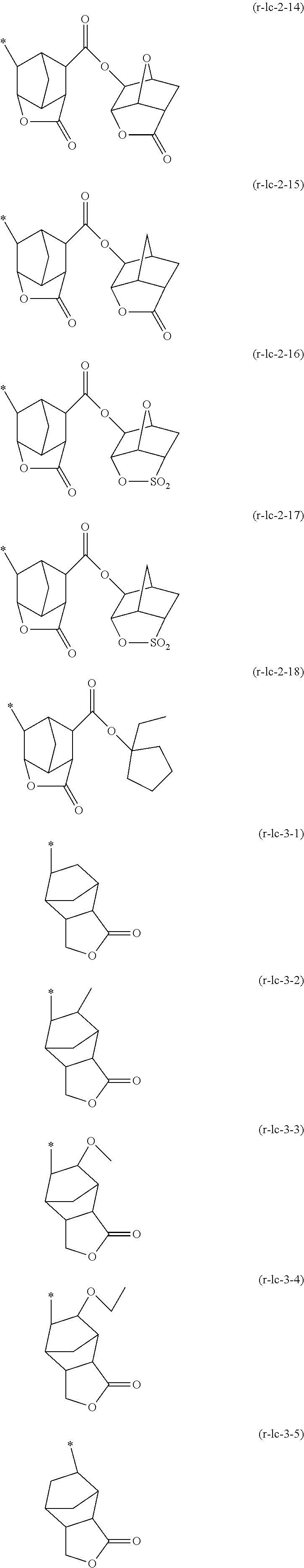

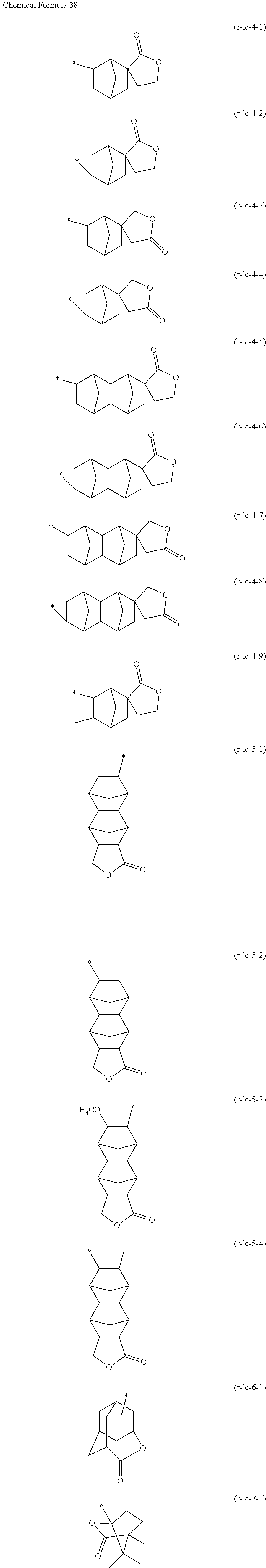

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

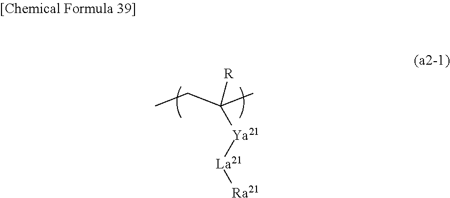

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a heteroatom.

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²¹ represents —COO— or —OCO—.

In Formula (a2-1), Ra²¹ represents a lactone-containing cyclic group. Suitable examples of the lactone-containing cyclic group as Rau include groups each represented by General Formulae (a2-r-1) to (a2-r-7).

The constitutional unit (a2) included in the component (A1) may be used alone or two or more kinds thereof.

In a case where the component (A1) has the constitutional unit (a2), the proportion of the constitutional unit (a2) is preferably 20% by mole or less, more preferably in a range of 1% to 20% by mole, still more preferably in a range of 1% to 15% by mole, and particularly preferably in a range of 1% to 10% by mole with respect to the total amount (100% by mole) of all constitutional units constituting the component (A1).

In a case where the proportion of the constitutional unit (a2) is set to be greater than or equal to the above-described preferable lower limits, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and various lithography characteristics are enhanced.

### In Regard to Constitutional Unit (a8):

The constitutional unit (a8) is a constitutional unit derived from a compound represented by General Formula (a8-1).

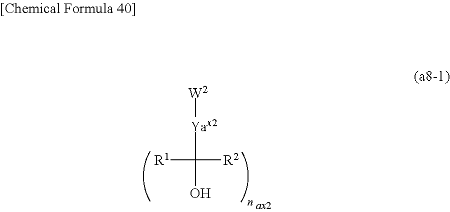

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms, which may have a fluorine atom, or a hydrogen atom. R² and Ya^{x2} may be bonded to each other to form a ring structure by being bonded to each other. nₐₓ₂ represents an integer of 1 to 3.]

The term “polymerizable group” in the polymerizable group-containing group as W² is a group that enables a compound having the polymerizable group to be polymerized by radical polymerization or the like, and refers to a group containing multiple bonds between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by a chemical formula: C(R^{X11})(R^{X12})═C(R^{X13})-Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

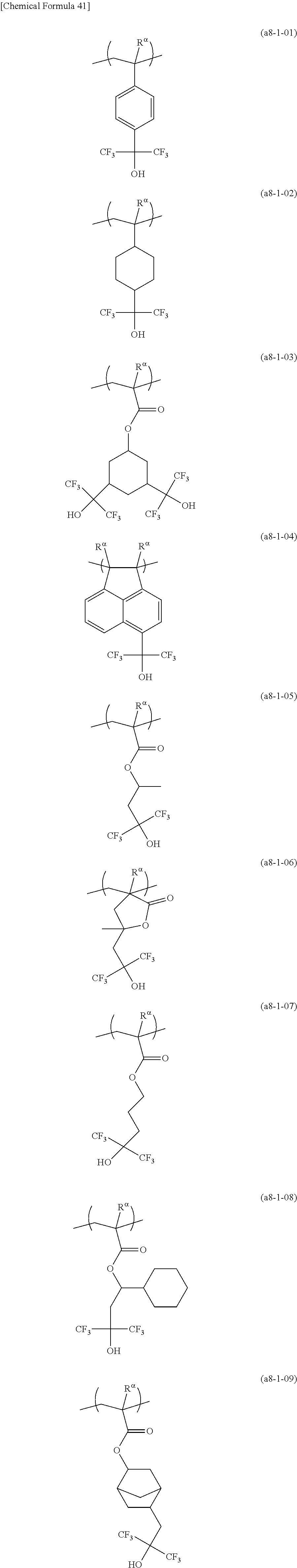

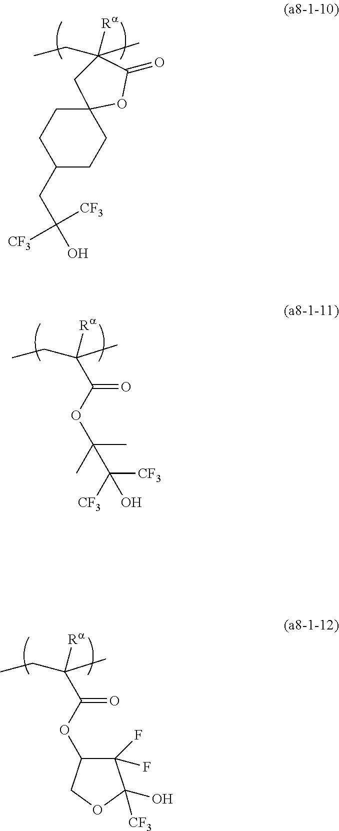

The constitutional unit (a8) contained in the component (A1) may be used alone or two or more kinds thereof.

In a case where the component (A1) has the constitutional unit (a8), the proportion of the constitutional unit (a8) is preferably 20% by mole or less and more preferably in a range of 0% to 10% by mole with respect to the total amount (100% by mole) of all constitutional units constituting the component (A1).

The component (A1) contained in the resist composition may be used alone or in combination of two or more kinds thereof.

In the resist composition of the present embodiment, preferred examples of the component (A1) include a polymer compound having a repeating structure of the constitutional unit (a1). Among the examples, particularly suitable examples of the component (A1) include a polymer compound having a repeating structure of the constitutional unit (a1) and the constitutional unit (a10).

More specifically, preferred examples of the component (A1) include a polymer compound having a repeating structure of a constitutional unit represented by Formula (a1-1) and the constitutional unit (a10), and a polymer compound having a repeating structure of a constitutional unit represented by Formula (a1-3) and the constitutional unit (a10). Among these, from the viewpoint of high sensitivity, a polymer compound having a repeating structure of a constitutional unit represented by Formula (a1-3) and the constitutional unit (a10) is more preferable.

The proportion of the constitutional unit (a1) in the polymer compound having a repeating structure of the constitutional unit (a1) and the constitutional unit (a10) is preferably in a range of 20% to 80% by mole, more preferably in a range of 25% to 75% by mole, still more preferably in a range of 30% to 70% by mole, and particularly preferably in a range of 40% to 60% by mole with respect to the total amount (100% by mole) of all constitutional units constituting the polymer compound.

In addition, the proportion of the constitutional unit (a10) in the polymer compound is preferably in a range of 20% to 80% by mole, more preferably in a range of 25% to 75% by mole, still more preferably in a range of 30% to 70% by mole, and particularly preferably in a range of 40% to 60% by mole with respect to the total amount (100% by mole) of all constitutional units constituting the polymer compound.

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, the component (A1) can be produced by dissolving, in a polymerization solvent, a monomer from which the constitutional unit (a1) is derived and, as necessary, a monomer from which a constitutional unit (for example, the constitutional unit (a10)) other than the constitutional unit (a1) is derived, adding thereto a radical polymerization initiator as described above to carry out polymerization, and then carrying out a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 40000, more preferably in a range of 2000 to 20000, and still more preferably in a range of 3000 to 10000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are satisfactory.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having various excellent lithography characteristics such as high sensitivity, resolution, and roughness reduction is likely to be formed.

In the resist composition of the present embodiment, the content of the component (A) may be adjusted according to the thickness and the like of the resist film intended to be formed.

### <Component (D): Base Component>

In the present embodiment, the component (D) includes a compound (D0) represented by General Formula (d0) (hereinafter, also referred to as “component (D0)”). The component (D0) is a component that can act as a quencher (acid diffusion control agent) which traps the acid generated in the resist composition upon light exposure.

### In Regard to Component (D0)

The component (D0) is a compound represented by General Formula (d0).

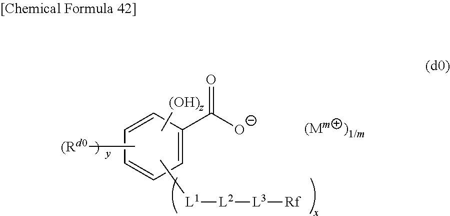

[In the formula, Rf represents a hydrocarbon group which may have a fluorine atom. L² represents a divalent cyclic group which may have a substituent. L¹ and L³ each independently represent a divalent linking group. x represents an integer of 1 to 4. In a case where x represents an integer of 2 or greater, a plurality of (-L¹-L²-L³-Rf)'s may be the same as or different from each other. R^{d0} represents an iodine atom or a bromine atom. y represents an integer of 1 to 4. In a case where y represents an integer of 2 or greater, a plurality of R^{d}0's may be the same as or different from each other. z represents an integer of 0 to 3. Here, 2≤x+y+z≤5 is satisfied. (M^{m+})_{1/m} represents a counter cation. m represents an integer of 1 or greater.]

### {Anion Moiety of Component (D0)}

In Formula (d0), Rf represents a hydrocarbon group which may have a fluorine atom.

The hydrocarbon group as Rf may be an aliphatic hydrocarbon group or an aromatic hydrocarbon group.

### Aliphatic Hydrocarbon Group as Rf

The aliphatic hydrocarbon group may be saturated or unsaturated, but is preferably saturated. Examples of the aliphatic hydrocarbon group include a chain-like aliphatic hydrocarbon group (linear or branched aliphatic hydrocarbon group) and an aliphatic hydrocarbon group having a ring in the structure.

### Linear or Branched Aliphatic Hydrocarbon Group

The linear aliphatic hydrocarbon group has preferably 1 to 10 carbon atoms, more preferably 1 to 8 carbon atoms, and still more preferably 2 to 6 carbon atoms. As the linear aliphatic hydrocarbon group, a linear alkyl group is preferable.

The branched aliphatic hydrocarbon group has preferably 3 to 10 carbon atoms and more preferably 3 to 6 carbon atoms. The branched aliphatic hydrocarbon group is preferably a branched alkyl group.

The linear or branched aliphatic hydrocarbon group may have a fluorine atom as a substituent and preferably has a fluorine atom as a substituent from the viewpoint of the pattern shape (rectangularity). In the linear or branched aliphatic hydrocarbon group, it is preferable that 25% or greater of hydrogen atoms in the aliphatic hydrocarbon group are fluorinated, more preferable that 50% or greater of hydrogen atoms in the aliphatic hydrocarbon group are fluorinated, and still more preferable that 60% or greater of hydrogen atoms in the aliphatic hydrocarbon group are fluorinated.

The linear or branched aliphatic hydrocarbon group may have a substituent other than a fluorine atom. Examples of the substituent other than the fluorine atom include a halogen atom other than a fluorine atom.

### Aliphatic Hydrocarbon Group Containing Ring in Structure Thereof

Examples of the aliphatic hydrocarbon group having a ring in the structure thereof include a cyclic aliphatic hydrocarbon group which may have a substituent having a heteroatom (excluding a fluorine atom) in the ring structure thereof (a group in which one hydrogen atom has been removed from an aliphatic hydrocarbon ring), a group in which the cyclic aliphatic hydrocarbon group is bonded to a terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group.

As the monocyclic alicyclic hydrocarbon group, a group in which one hydrogen atom has been removed from a monocycloalkane is preferable. The monocycloalkane has preferably 3 to 6 carbon atoms, and specific examples thereof include cyclopentane and cyclohexane.

As the polycyclic alicyclic hydrocarbon group, a group in which one hydrogen atom has been removed from a polycycloalkane is preferable. As the polycycloalkane, a group having 7 to 12 carbon atoms is preferable, and specific examples of the polycycloalkane include adamantane, norbornane, isobornane, tricyclodecane, and tetracyclododecane.

The cyclic aliphatic hydrocarbon group may have a fluorine atom or a fluorinated hydrocarbon group as a substituent, and preferably has a fluorine atom or a fluorinated hydrocarbon group as a substituent from the viewpoint of the pattern shape (rectangularity). The fluorinated hydrocarbon group as a substituent here is preferably a fluorinated alkyl group having 1 to 5 carbon atoms and more preferably a fluorinated alkyl group having 1 to 3 carbon atoms.

In the cyclic aliphatic hydrocarbon group, it is preferable that 25% or greater of hydrogen atoms in the cyclic aliphatic hydrocarbon group are fluorinated, more preferable that 50% or greater of hydrogen atoms in the cyclic aliphatic hydrocarbon group are fluorinated, and still more preferable that 60% or greater of hydrogen atoms in the cyclic aliphatic hydrocarbon group are fluorinated.

The cyclic aliphatic hydrocarbon group may have a substituent other than a fluorine atom and a fluorinated hydrocarbon group. Examples of the substituent other than a fluorine atom and a fluorinated hydrocarbon group include a halogen atom other than a fluorine atom and an alkyl group having 1 to 5 carbon atoms.

### Aromatic Hydrocarbon Group as Rf

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include an aromatic hydrocarbon ring such as benzene, naphthalene, anthracene, or phenanthrene, and an aromatic heterocyclic ring in which some carbon atoms constituting the aromatic hydrocarbon ring are substituted with heteroatoms (excluding a fluorine atom). Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an aryl group or a heteroaryl group) obtained by removing one hydrogen atom from the aromatic hydrocarbon ring or aromatic heterocyclic ring, a group obtained by removing one hydrogen atom from an aromatic compound (for example, biphenyl or fluorene) having two or more aromatic rings, and a group (for example, an arylalkyl group such as a benzyl group, a phenethyl group, a 1-naphthylmethyl group, a 2-naphthylmethyl group, a 1-naphthylethyl group, or a 2-naphthylethyl group) in which one hydrogen atom in the aromatic hydrocarbon ring or aromatic heterocyclic ring is substituted with an alkylene group. The number of carbon atoms in the alkylene group bonded to the aromatic hydrocarbon ring or aromatic heterocyclic ring is preferably in a range of 1 to 4, more preferably 1 or 2, and particularly preferably 1.

The aromatic hydrocarbon group may have a fluorine atom or a fluorinated hydrocarbon group as a substituent, and preferably has a fluorine atom or a fluorinated hydrocarbon group as a substituent from the viewpoint of the pattern shape (rectangularity). The fluorinated hydrocarbon group as a substituent here is preferably a fluorinated alkyl group having 1 to 5 carbon atoms and more preferably a fluorinated alkyl group having 1 to 3 carbon atoms.

In the aromatic hydrocarbon group, it is preferable that 25% or greater of hydrogen atoms in the aromatic hydrocarbon group are fluorinated, more preferable that 50% or greater of hydrogen atoms in the aromatic hydrocarbon group are fluorinated, and still more preferable that 60% or greater of hydrogen atoms in the aromatic hydrocarbon group are fluorinated.

The aromatic hydrocarbon group may have a substituent other than a fluorine atom and a fluorinated hydrocarbon group. Examples of the substituent other than a fluorine atom and a fluorinated hydrocarbon group include a halogen atom other than a fluorine atom and an alkyl group having 1 to 5 carbon atoms.

From the viewpoint of easily controlling the distribution of the component (D0) in the resist film (uneven distribution on the surface of the resist film), Rf represents preferably a fluorinated hydrocarbon group, more preferably a chain-like aliphatic hydrocarbon group having a fluorine atom as a substituent or an aromatic hydrocarbon group having a fluorine atom or a fluorinated hydrocarbon group as a substituent, still more preferably a linear aliphatic hydrocarbon group having a fluorine atom as a substituent or an aromatic hydrocarbon group having a fluorine atom or a fluorinated alkyl group as a substituent, and particularly preferably a linear aliphatic hydrocarbon group having a fluorine atom as a substituent and 1 to 10 carbon atoms or an aromatic hydrocarbon group having a fluorine atom or a fluorinated alkyl group having 1 to 3 carbon atoms as a substituent.

In Formula (d0), x represents an integer of 1 to 4, preferably 1 or 2, and more preferably 1 from the viewpoint of synthesis.

In Formula (d0), the total number of fluorine atoms contained in x pieces of Rf's is preferably 5 or more, more preferably 5 or more and 11 or less, still more preferably 5 or more and 10 or less, and particularly preferably 5 or more and 9 or less.

In a case where the total number of fluorine atoms is in the above-described preferable ranges, lithography characteristics such as rectangularity of a pattern shape and roughness reduction are enhanced in the resist pattern formation.

In a case where x represents 2 or greater, the total number of fluorine atoms contained in Rf denotes the number obtained by adding all the fluorine atoms contained in 2 or more Rf's.

In Formula (d0), L² represents a divalent cyclic group which may have a substituent.

The divalent cyclic group as L² may be an aliphatic hydrocarbon group or an aromatic hydrocarbon group, and may also be a polycyclic group or a monocyclic group.

As the aliphatic hydrocarbon group of the monocyclic group as L², a group obtained by removing two or more hydrogen atoms from a monocycloalkane or monocycloalkene is preferable. The monocycloalkane has preferably 3 to 6 carbon atoms, and specific examples thereof include cyclopentane and cyclohexane. The monocycloalkene preferably has 3 to 6 carbon atoms, and specific examples thereof include cyclopentene and cyclohexene.

As the aliphatic hydrocarbon group of the polycyclic group as L², a group obtained by removing two or more hydrogen atoms from a polycycloalkane or polycycloalkene is preferable, a polycycloalkane having 7 to 12 carbon atoms is preferable as the polycycloalkane, and specific examples thereof include adamantane, norbornane, isobornane, tricyclodecane, and tetracyclododecane. In addition, the polycycloalkene is preferably a group having 7 to 12 carbon atoms. Specific examples thereof include adamantene, norbornene, isobornene, tricyclodecene, and tetracyclododecene.

As the aromatic hydrocarbon group as L², a group obtained by removing two or more hydrogen atoms from benzene, naphthalene, anthracene, or phenanthrene is preferable, a group obtained by removing two or more hydrogen atoms from benzene, naphthalene, or anthracene is more preferable, and a group obtained by removing two or more hydrogen atoms from benzene is still more preferable.

Examples of the substituent which may be contained in the divalent cyclic group as L² include the same groups as that for Ra^{x5} described above.

Further, in the cyclic group, some carbon atoms (methylene groups and the like) constituting the cyclic group may be substituted with heteroatom-containing groups.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

That is, the divalent cyclic group which may have a substituent as L² may be a group obtained by removing two or more hydrogen atoms from an aromatic heterocyclic ring such as a pyridine ring or a thiophene ring, or a group obtained by removing two or more hydrogen atoms from an aliphatic heterocyclic ring such as tetrahydrofuran, tetrahydropyran, tetrahydrothiophene, or a lactone ring.

The divalent cyclic group as L² may be a condensed cyclic group having a condensed ring in which an aliphatic hydrocarbon ring and an aromatic ring are condensed. Examples of the condensed ring include those obtained by condensing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed to bicycloalkane is preferable.

In General Formula (d0), among the examples, from the viewpoint of improving the dispersion uniformity of the component (D0) in the resist film, L² represents preferably an aromatic hydrocarbon group which may have a substituent or a polycyclic alicyclic hydrocarbon group which may have a substituent and more preferably an aromatic hydrocarbon group, a group obtained by removing two hydrogen atoms from a polycycloalkane or polycycloalkene, or a condensed cyclic group having a condensed ring in which an aliphatic hydrocarbon ring and an aromatic ring are condensed.

Specifically, L² represents preferably a group represented by any of Chemical Formulae (L²-1) to (L²-8), more preferably a group represented by any of Chemical Formulae (L²-1) to (L²-4), and still more preferably a group represented by any of Chemical Formulae (L²-2), (L²-3), or (L²-4).

Further, the hydrogen atoms of the groups each represented by Chemical Formulae (L²-1) to (L²-8) may be each independently substituted with a substituent. Examples of the substituent include the same substituents as those for Ra^{x}s described above.

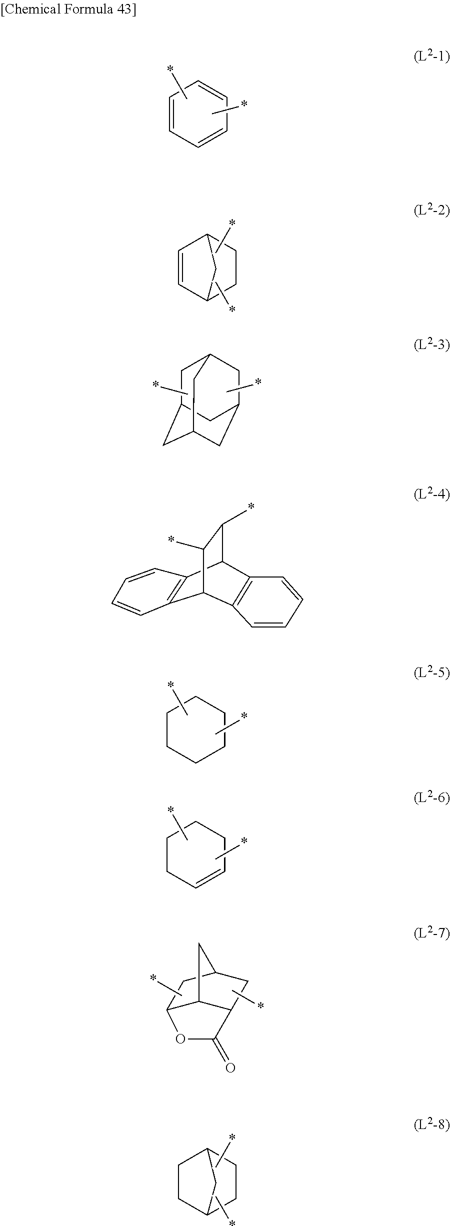

In Formula (d0), L¹ and L³ each independently represent a divalent linking group.

Suitable examples of the divalent linking group as L¹ and L³ include a divalent linking group having an oxygen atom.

In a case where L¹ and L³ represent a divalent linking group having an oxygen atom, L¹ and L³ may have an atom other than the oxygen atom. Examples of the atom other than the oxygen atom include a carbon atom, a hydrogen atom, a sulfur atom, and a nitrogen atom.

Examples of divalent linking groups containing an oxygen atom include non-hydrocarbon-based oxygen atom-containing linking groups such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon-based oxygen atom-containing linking groups with an alkylene group. Further, a sulfonyl group (—SO₂—) may be further linked to the combination.

More specific examples of the divalent linking group as L¹ and L³ include —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, —C(═O)—N(R^{a})—, and combinations of the above-described non-hydrocarbon-based oxygen atom-containing linking groups with an alkylene group. R^{a}'s each independently represent a hydrogen atom or an alkyl group.

In Formula (d0), L¹ represents preferably a divalent linking group having an oxygen atom, more preferably —OCO—, —COO—, or —C(═O)—N(R^{a})—, and particularly preferably —OCO—, —COO—, —NH—C(═O)—, or —C(═O)—NH—.

In Formula (d0), L³ represents preferably a divalent linking group having an oxygen atom and more preferably —OCO—, —COO—, —NH—C(═O)—, —C(═O)—NH—, or —O—C(═O)-alkylene group-O—.

In Formula (d0), R^{d0} represents an iodine atom or a bromine atom and preferably an iodine atom from the viewpoint of more easily increasing the sensitivity. y represents an integer of 1 to 4, preferably 1, 2, or 3 from the viewpoint of synthesis, and more preferably 2 or 3 from the viewpoint of achieving high sensitivity. z represents an integer of 0 to 3, and may be 0, 1, or 2, or may be 0 or 1. From the viewpoints of the solubility in a developing solution and the effect of reducing the roughness, z represents preferably 1 or 2 and more preferably 1.

Specific preferred examples of the anion moiety of the component (D0) are shown below.

Specific examples of the anion moiety in a case where y in Formula (d0) represents 1 include anions each represented by Chemical Formulae (d0-an-101) to (d0-an-113).

Specific examples of the anion moiety in a case where y in Formula (d0) represents 2 include anions each represented by Chemical Formulae (d0-an-201) to (d0-an-242).

Specific examples of the anion moiety in a case where y in Formula (d0) represents 3 include anions each represented by Chemical Formulae (d0-an-301) to (d0-an-310).

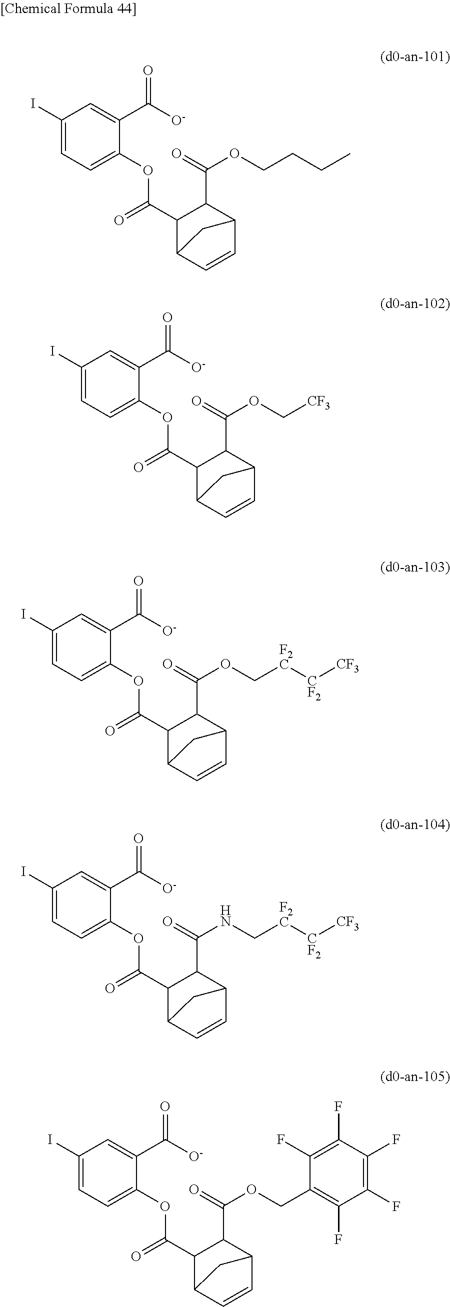 [SMILES: CCCCOC(=O)C1C2C=CC(C2)C1C(=O)Oc1ccc(I)cc1C(=O)[O-] ; O=C([O-])c1cc(I)ccc1OC(=O)C1C2C=CC(C2)C1C(=O)OCC(F)(F)F ; O=C([O-])c1cc(I)ccc1OC(=O)C1C2C=CC(C2)C1C(=O)OCC(F)(F)C(F)(F)C(F)(F)F ; O=C([O-])c1cc(I)ccc1OC(=O)C1C2C=CC(C2)C1C(=O)NCC(F)(F)C(F)(F)C(F)(F)F ; O=C([O-])c1cc(I)ccc1OC(=O)C1C2C=CC(C2)C1C(=O)OCc1c(F)c(F)c(F)c(F)c1F]

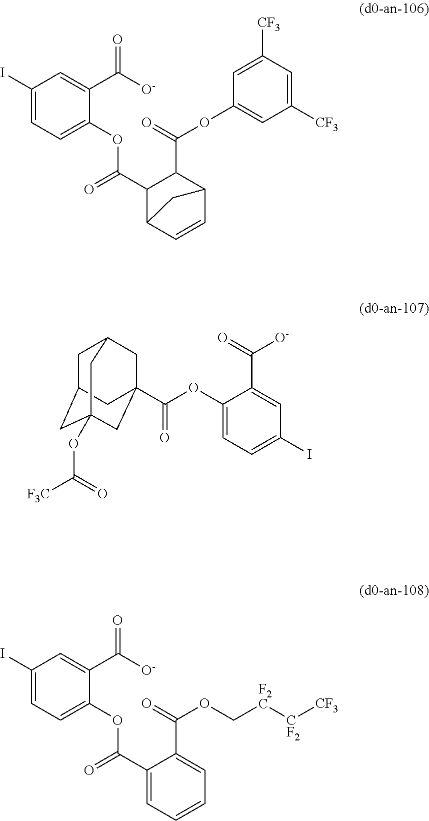 [SMILES: O=C([O-])c1cc(I)ccc1OC(=O)C1C2C=CC(C2)C1C(=O)Oc1cc(C(F)(F)F)cc(C(F)(F)F)c1 ; O=C([O-])c1cc(I)ccc1OC(=O)C12CC3CC(CC(OC(=O)C(F)(F)F)(C3)C1)C2 ; O=C([O-])c1cc(I)ccc1OC(=O)c1ccccc1C(=O)OCC(F)(F)C(F)(F)C(F)(F)F]

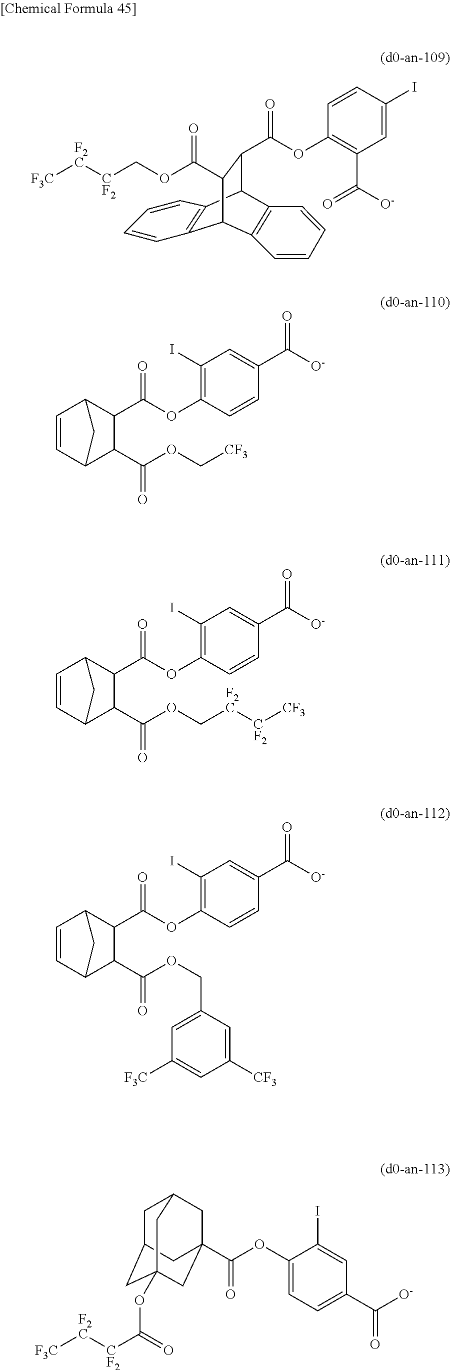 [SMILES: O=C([O-])c1cc(I)ccc1OC(=O)C1C2c3ccccc3C(c3ccccc32)C1C(=O)OCC(F)(F)C(F)(F)C(F)(F)F ; O=C([O-])c1ccc(OC(=O)C2C3C=CC(C3)C2C(=O)OCC(F)(F)F)c(I)c1 ; O=C([O-])c1ccc(OC(=O)C2C3C=CC(C3)C2C(=O)OCC(F)(F)C(F)(F)C(F)(F)F)c(I)c1 ; O=C([O-])c1ccc(OC(=O)C2C3C=CC(C3)C2C(=O)OCc2cc(C(F)(F)F)cc(C(F)(F)F)c2)c(I)c1 ; O=C([O-])c1ccc(OC(=O)C23CC4CC(CC(OC(=O)C(F)(F)C(F)(F)C(F)(F)F)(C4)C2)C3)c(I)c1]

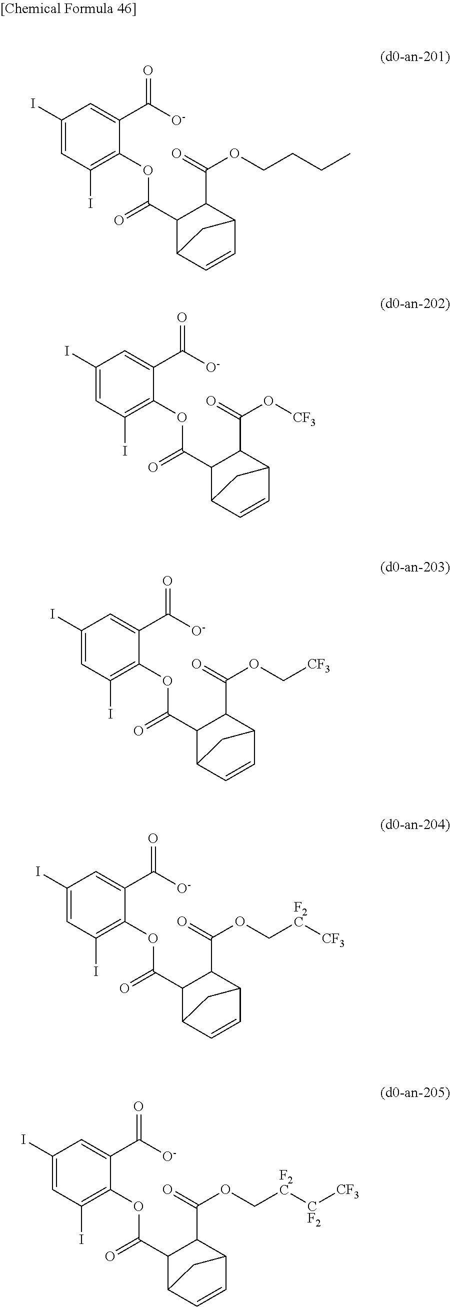 [SMILES: CCCCOC(=O)C1C2C=CC(C2)C1C(=O)Oc1c(I)cc(I)cc1C(=O)[O-] ; O=C([O-])c1cc(I)cc(I)c1OC(=O)C1C2C=CC(C2)C1C(=O)OC(F)(F)F ; O=C([O-])c1cc(I)cc(I)c1OC(=O)C1C2C=CC(C2)C1C(=O)OCC(F)(F)F ; O=C([O-])c1cc(I)cc(I)c1OC(=O)C1C2C=CC(C2)C1C(=O)OCC(F)(F)C(F)(F)F ; O=C([O-])c1cc(I)cc(I)c1OC(=O)C1C2C=CC(C2)C1C(=O)OCC(F)(F)C(F)(F)C(F)(F)F]

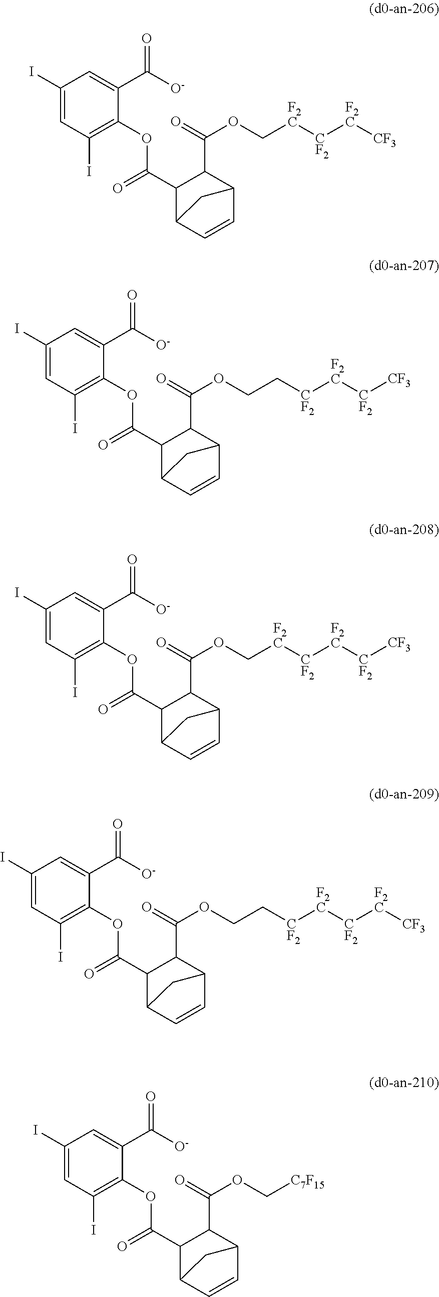

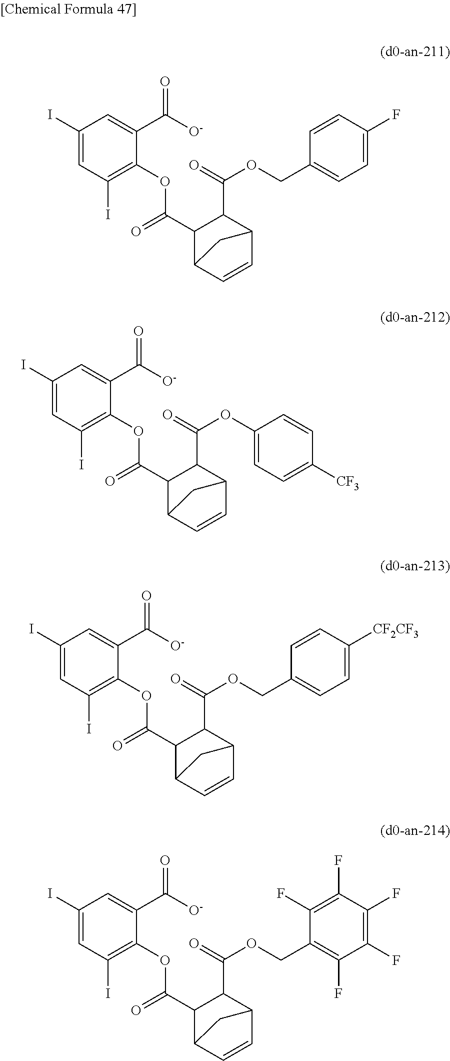

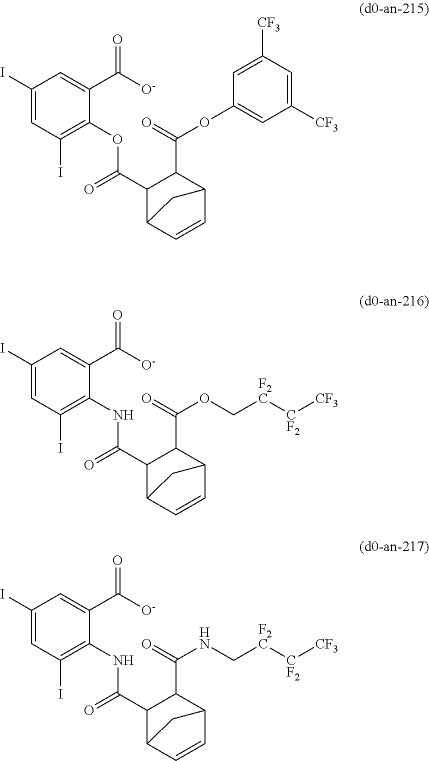 [SMILES: O=C([O-])c1cc(I)cc(I)c1OC(=O)C1C2C=CC(C2)C1C(=O)Oc1cc(C(F)(F)F)cc(C(F)(F)F)c1 ; O=C([O-])c1cc(I)cc(I)c1NC(=O)C1C2C=CC(C2)C1C(=O)OCC(F)(F)C(F)(F)C(F)(F)F ; O=C([O-])c1cc(I)cc(I)c1NC(=O)C1C2C=CC(C2)C1C(=O)NCC(F)(F)C(F)(F)C(F)(F)F]

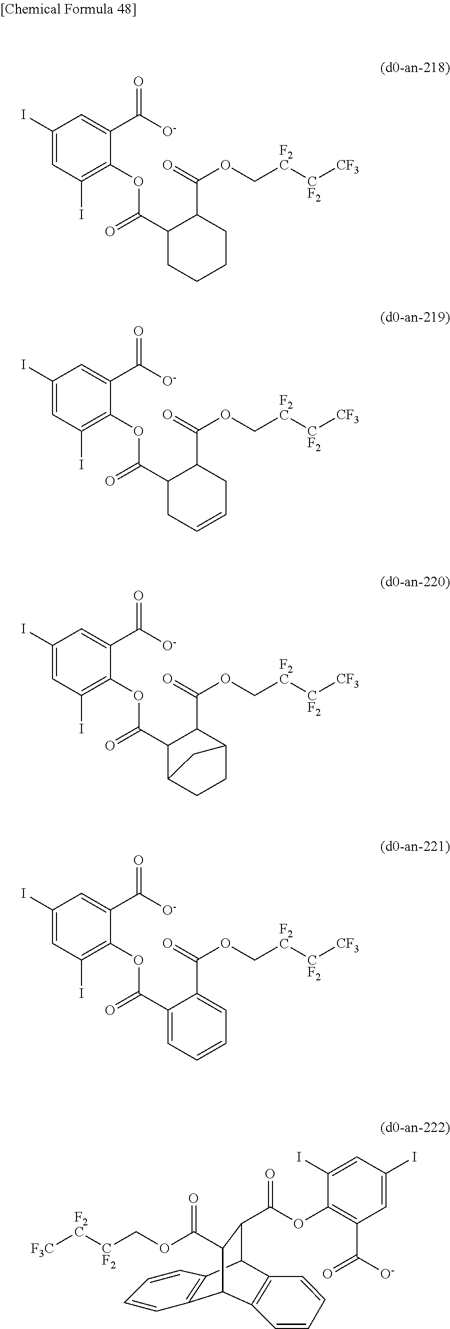 [SMILES: O=C([O-])c1cc(I)cc(I)c1OC(=O)C1CCCCC1C(=O)OCC(F)(F)C(F)(F)C(F)(F)F ; O=C([O-])c1cc(I)cc(I)c1OC(=O)C1CC=CCC1C(=O)OCC(F)(F)C(F)(F)C(F)(F)F ; O=C([O-])c1cc(I)cc(I)c1OC(=O)C1C2CCC(C2)C1C(=O)OCC(F)(F)C(F)(F)C(F)(F)F ; O=C(OCC(F)(F)C(F)(F)C(F)(F)F)c1ccccc1C(=O)Oc1c(I)cc(I)cc1C(=O)[O-] ; O=C([O-])c1cc(I)cc(I)c1OC(=O)C1C2c3ccccc3C(c3ccccc32)C1C(=O)OCC(F)(F)C(F)(F)C(F)(F)F]

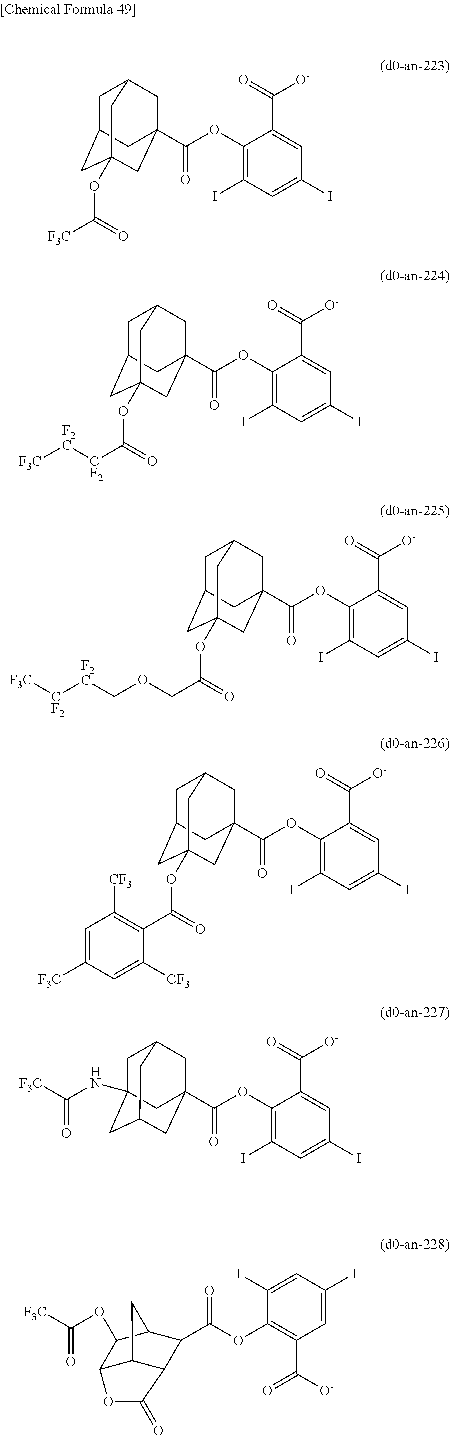 [SMILES: O=C([O-])c1cc(I)cc(I)c1OC(=O)C12CC3CC(CC(OC(=O)C(F)(F)F)(C3)C1)C2 ; O=C([O-])c1cc(I)cc(I)c1OC(=O)C12CC3CC(CC(OC(=O)C(F)(F)C(F)(F)C(F)(F)F)(C3)C1)C2 ; O=C(COCC(F)(F)C(F)(F)C(F)(F)F)OC12CC3CC(C1)CC(C(=O)Oc1c(I)cc(I)cc1C(=O)[O-])(C3)C2 ; O=C([O-])c1cc(I)cc(I)c1OC(=O)C12CC3CC(CC(OC(=O)c4c(C(F)(F)F)cc(C(F)(F)F)cc4C(F)(F)F)(C3)C1)C2 ; O=C([O-])c1cc(I)cc(I)c1OC(=O)C12CC3CC(CC(NC(=O)C(F)(F)F)(C3)C1)C2 ; O=C([O-])c1cc(I)cc(I)c1OC(=O)C1C2CC3C(OC(=O)C31)C2OC(=O)C(F)(F)F]

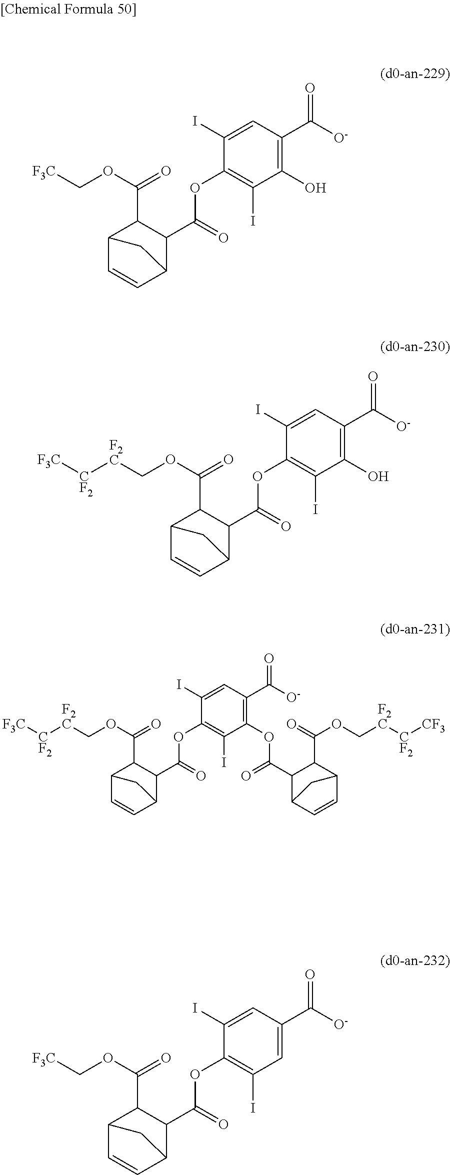 [SMILES: O=C([O-])c1cc(I)c(OC(=O)C2C3C=CC(C3)C2C(=O)OCC(F)(F)F)c(I)c1O ; O=C([O-])c1cc(I)c(OC(=O)C2C3C=CC(C3)C2C(=O)OCC(F)(F)C(F)(F)C(F)(F)F)c(I)c1O ; O=C([O-])c1cc(I)c(OC(=O)C2C3C=CC(C3)C2C(=O)OCC(F)(F)C(F)(F)C(F)(F)F)c(I)c1OC(=O)C1C2C=CC(C2)C1C(=O)OCC(F)(F)C(F)(F)C(F)(F)F ; O=C([O-])c1cc(I)c(OC(=O)C2C3C=CC(C3)C2C(=O)OCC(F)(F)F)c(I)c1]

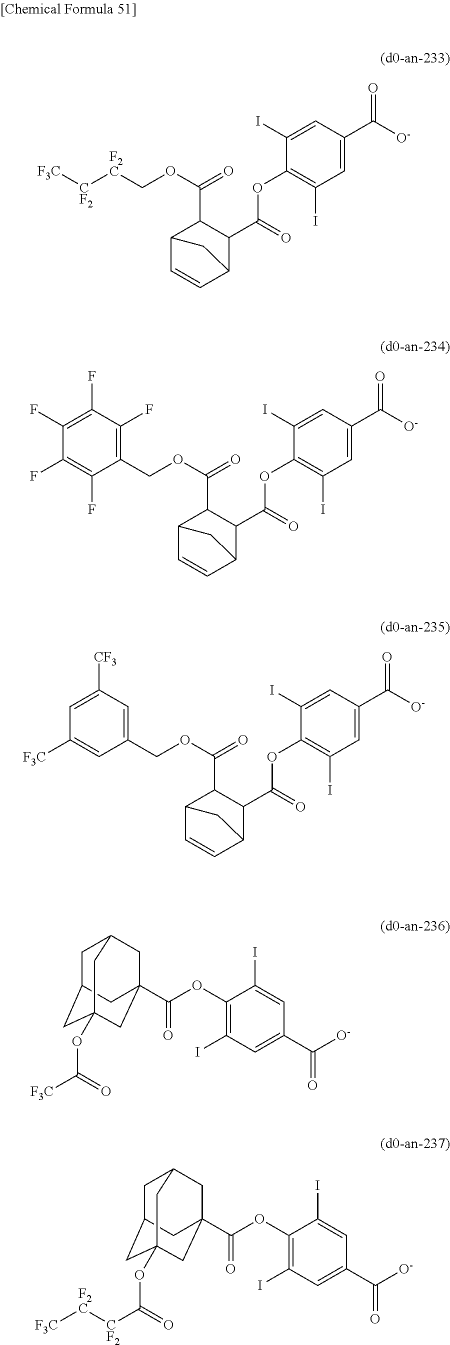 [SMILES: O=C([O-])c1cc(I)c(OC(=O)C2C3C=CC(C3)C2C(=O)OCC(F)(F)C(F)(F)C(F)(F)F)c(I)c1 ; O=C([O-])c1cc(I)c(OC(=O)C2C3C=CC(C3)C2C(=O)OCc2c(F)c(F)c(F)c(F)c2F)c(I)c1 ; O=C([O-])c1cc(I)c(OC(=O)C2C3C=CC(C3)C2C(=O)OCc2cc(C(F)(F)F)cc(C(F)(F)F)c2)c(I)c1 ; O=C([O-])c1cc(I)c(OC(=O)C23CC4CC(CC(OC(=O)C(F)(F)F)(C4)C2)C3)c(I)c1 ; O=C([O-])c1cc(I)c(OC(=O)C23CC4CC(CC(OC(=O)C(F)(F)C(F)(F)C(F)(F)F)(C4)C2)C3)c(I)c1]

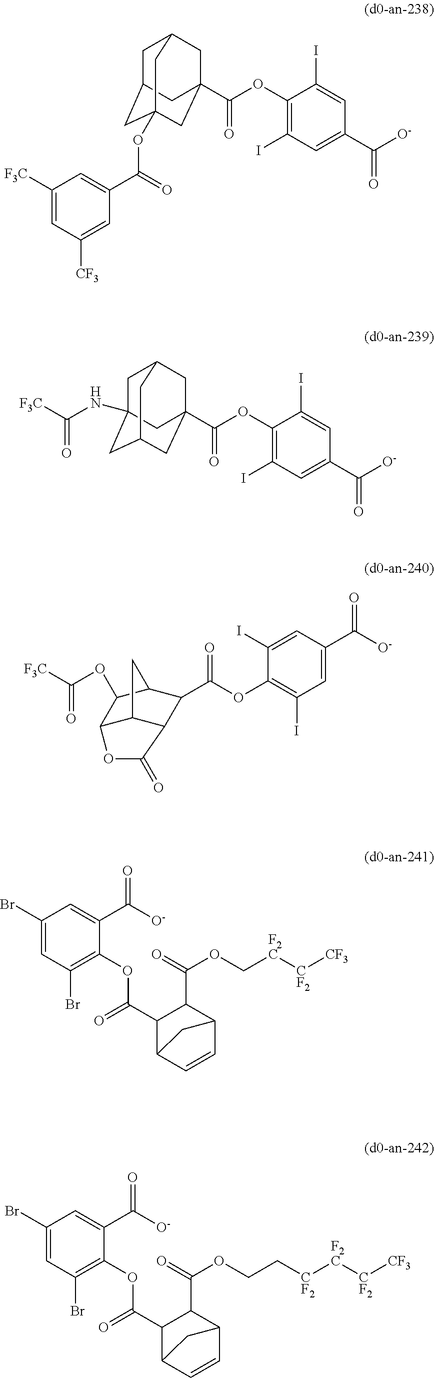 [SMILES: O=C([O-])c1cc(I)c(OC(=O)C23CC4CC(CC(OC(=O)c5cc(C(F)(F)F)cc(C(F)(F)F)c5)(C4)C2)C3)c(I)c1 ; O=C([O-])c1cc(I)c(OC(=O)C23CC4CC(CC(NC(=O)C(F)(F)F)(C4)C2)C3)c(I)c1 ; O=C([O-])c1cc(I)c(OC(=O)C2C3CC4C(OC(=O)C42)C3OC(=O)C(F)(F)F)c(I)c1 ; O=C([O-])c1cc(Br)cc(Br)c1OC(=O)C1C2C=CC(C2)C1C(=O)OCC(F)(F)C(F)(F)C(F)(F)F ; O=C([O-])c1cc(Br)cc(Br)c1OC(=O)C1C2C=CC(C2)C1C(=O)OCCC(F)(F)C(F)(F)C(F)(F)C(F)(F)F]

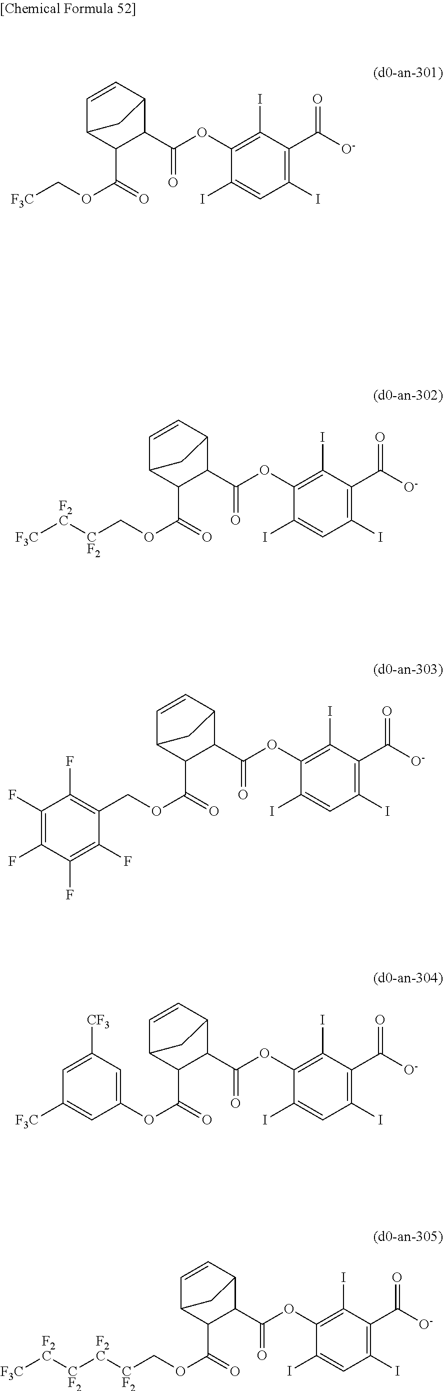 [SMILES: O=C([O-])c1c(I)cc(I)c(OC(=O)C2C3C=CC(C3)C2C(=O)OCC(F)(F)F)c1I ; O=C([O-])c1c(I)cc(I)c(OC(=O)C2C3C=CC(C3)C2C(=O)OCC(F)(F)C(F)(F)C(F)(F)F)c1I ; O=C([O-])c1c(I)cc(I)c(OC(=O)C2C3C=CC(C3)C2C(=O)OCc2c(F)c(F)c(F)c(F)c2F)c1I ; O=C([O-])c1c(I)cc(I)c(OC(=O)C2C3C=CC(C3)C2C(=O)Oc2cc(C(F)(F)F)cc(C(F)(F)F)c2)c1I ; O=C([O-])c1c(I)cc(I)c(OC(=O)C2C3C=CC(C3)C2C(=O)OCC(F)(F)C(F)(F)C(F)(F)C(F)(F)C(F)(F)F)c1I]

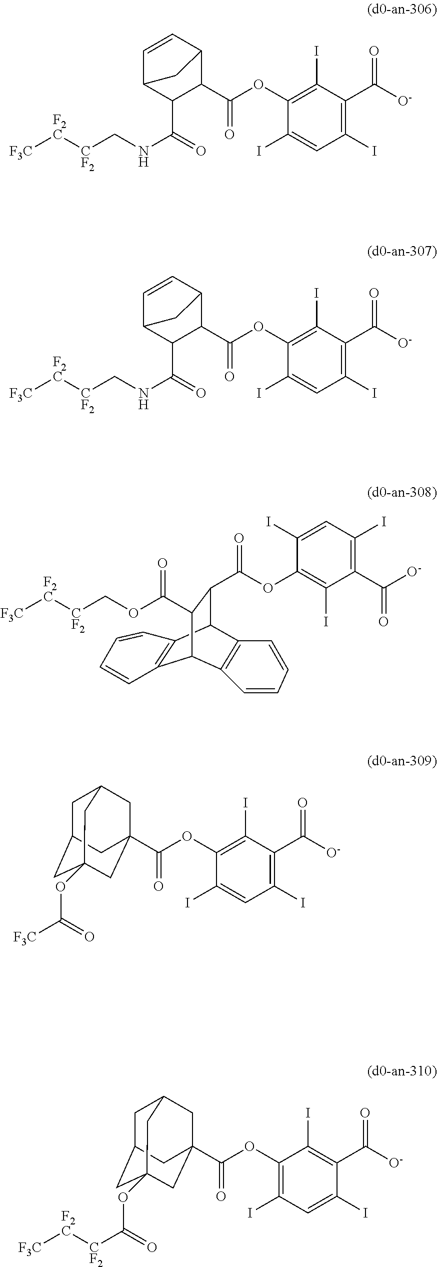 [SMILES: O=C([O-])c1c(I)cc(I)c(OC(=O)C2C3C=CC(C3)C2C(=O)NCC(F)(F)C(F)(F)C(F)(F)F)c1I ; O=C([O-])c1c(I)cc(I)c(OC(=O)C2C3C=CC(C3)C2C(=O)NCC(F)(F)C(F)(F)C(F)(F)F)c1I ; O=C([O-])c1c(I)cc(I)c(OC(=O)C2C3c4ccccc4C(c4ccccc43)C2C(=O)OCC(F)(F)C(F)(F)C(F)(F)F)c1I ; O=C([O-])c1c(I)cc(I)c(OC(=O)C23CC4CC(CC(OC(=O)C(F)(F)F)(C4)C2)C3)c1I ; O=C([O-])c1c(I)cc(I)c(OC(=O)C23CC4CC(CC(OC(=O)C(F)(F)C(F)(F)C(F)(F)F)(C4)C2)C3)c1I]

As the anion moiety of the component (D0), at least one selected from the group consisting of anions each represented by Chemical Formulae (d0-an-101) to (d0-an-113), Chemical Formulae (d0-an-201) to (d0-an-242), and Chemical Formulae (d0-an-301) to (d0-an-310) is preferable.

Among these, as the anion moiety of the component (D0), from the viewpoint of roughness reduction and the pattern shape (rectangularity), an anion corresponding to an anion in which the total number of fluorine atoms contained in Rf in General Formula (d0) is 5 or more and 9 or less is preferable.

Alternatively, among the examples, as the anion moiety of the component (D0), from the viewpoint of achieving high sensitivity, an anion corresponding to an anion in which R^{d0} in General Formula (d0) represents an iodine atom and y represents 2 or 3 is preferable, and an anion represented by any of Chemical Formulae (d0-an-201) to (d0-an-240) and an anion represented by any of Chemical Formulae (d0-an-301) to (d0-an-310) are more preferable.

Alternatively, among the examples, as the anion moiety of the component (D0), from the viewpoints of high sensitivity, roughness reduction, and the pattern shape (rectangularity), an anion corresponding to an anion in which R^{d0} in General Formula (d0) represents an iodine atom, y represents 2 or 3, and the total number of fluorine atoms contained in Rf is 5 or more and 9 or less is particularly preferable.

### {Cation Moiety of Component (D0)}

In Formula (d0), (M^{m+})_{1/m} represents a counter cation, and M^{m+} represents an m-valent cation. m represents an integer of 1 or greater.

Preferred examples of the cation moiety ((M^{m+})_{1/m}) include an onium cation. Among the examples, a sulfonium cation and an iodonium cation are preferable, and an onium cation represented by any of General Formulae (ca-1) to (ca-3) is more preferable.

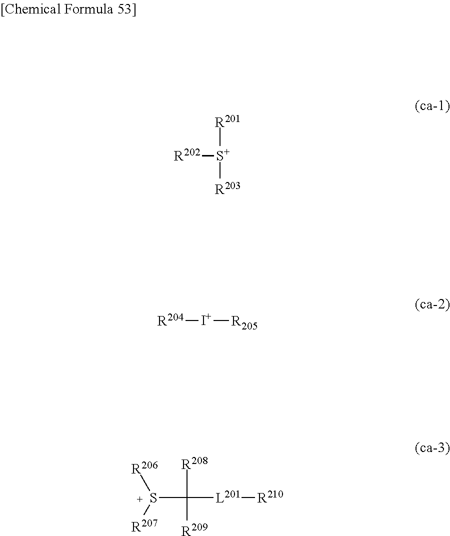

[In the formulae, R²⁰¹ to R²⁰⁷ each independently represent an aryl group, an alkyl group, or an alkenyl group, each of which may have a substituent. R²⁰¹ to R²⁰³ and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms, where a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent which may be contained in R²⁰¹ to R²⁰⁷ and R²¹⁰ include an alkyl group, a halogen atom, a halogenated alkyl group, a carbonyl group, a cyano group, an amino group, an aryl group, and groups each represented by General Formulae (ca-r-1) to (ca-r-7).

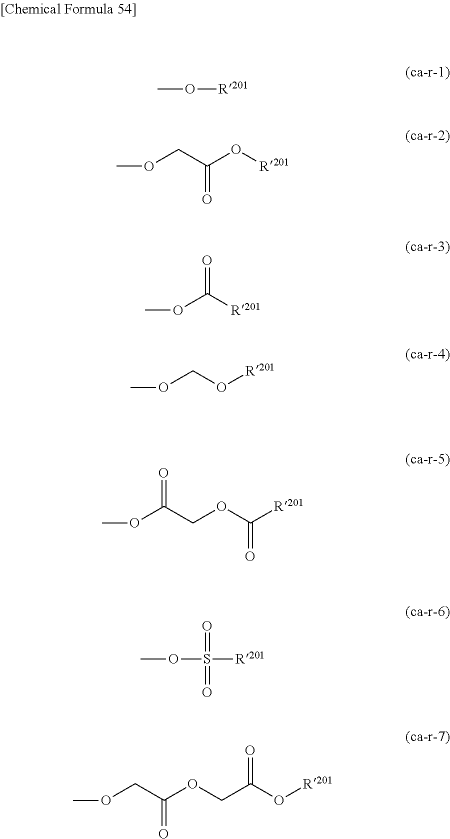

[In the formulae, R′²⁰¹'s each independently represents a hydrogen atom, a cyclic group which may have a substituent, a chain-like alkyl group which may have a substituent, or a chain-like alkenyl group which may have a substituent.]
   Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring-based polycyclic skeleton such as adamantane, norbornane, isobornane, tricyclodecane, or tetracyclododecane; and a polycycloalkane having a condensed ring-based polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Examples thereof include a lactone-containing cyclic group, an —SO₂-containing cyclic group, and another heterocyclic group represented by any of Chemical Formulae (r-hr-1) to (r-hr-16).

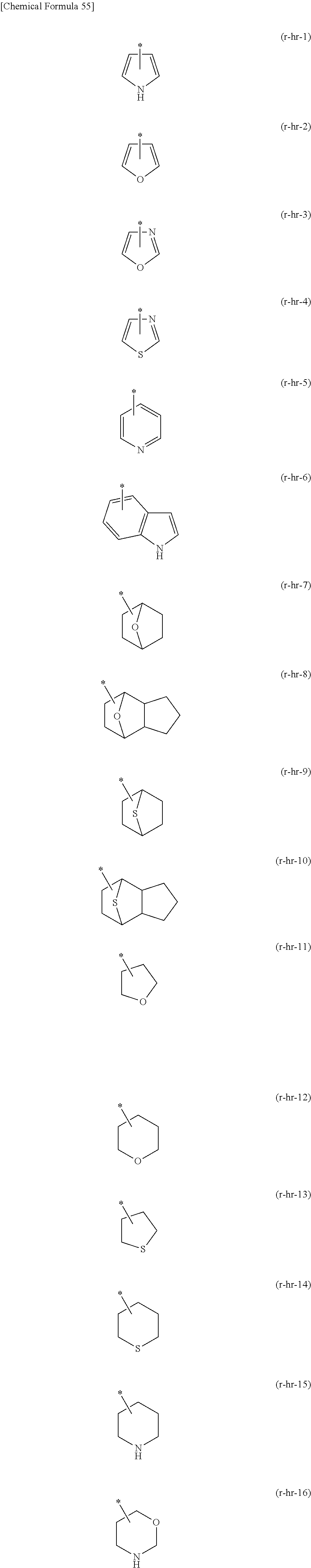

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹

Examples of the cyclic group which may have a substituent, the chain-like alkyl group which may have a substituent, and the chain-like alkenyl group which may have a substituent as R′²⁰¹ include the same groups as those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples include a group obtained by removing one or more hydrogen atoms from a phenyl group, a naphthyl group, or a polycycloalkane, a lactone-containing cyclic group, and a —SO₂-containing cyclic group.

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms, and a phenyl group or a naphthyl group is preferable. As the alkyl group represented by R²¹⁰, a chain-like or cyclic alkyl group having 1 to 30 carbon atoms is preferable.

The alkenyl group as R²¹⁰ preferably has 2 to 10 carbon atoms.

The —SO₂-containing cyclic group as R²¹⁰ is not particularly limited, and any —SO₂-containing cyclic group can be used. Specific examples thereof include groups each represented by General Formulae (b5-r-1) to (b5-r-4), where “—SO₂-containing polycyclic group” is preferable, and a group represented by General Formula (b5-r-1) is more preferable.

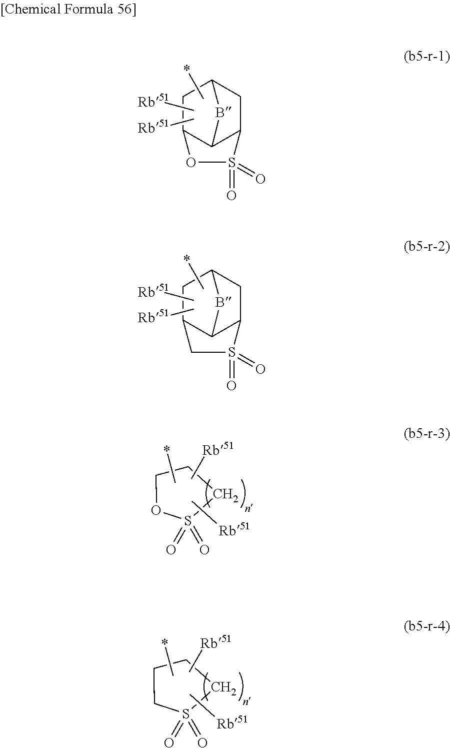

[In the formulae, Rb′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂-containing cyclic group; B″ represents an oxygen atom, a sulfur atom, or an alkylene group having 1 to 5 carbon atoms, which may contain an oxygen atom or a sulfur atom; and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, and among the examples, it is preferable that Rb′⁵¹'s each independently represent a hydrogen atom or a cyano group.

Specific examples of the groups each represented by General Formulae (b5-r-1) to (b5-r-4) are shown below. In the formulae shown below, “Ac” represents an acetyl group.

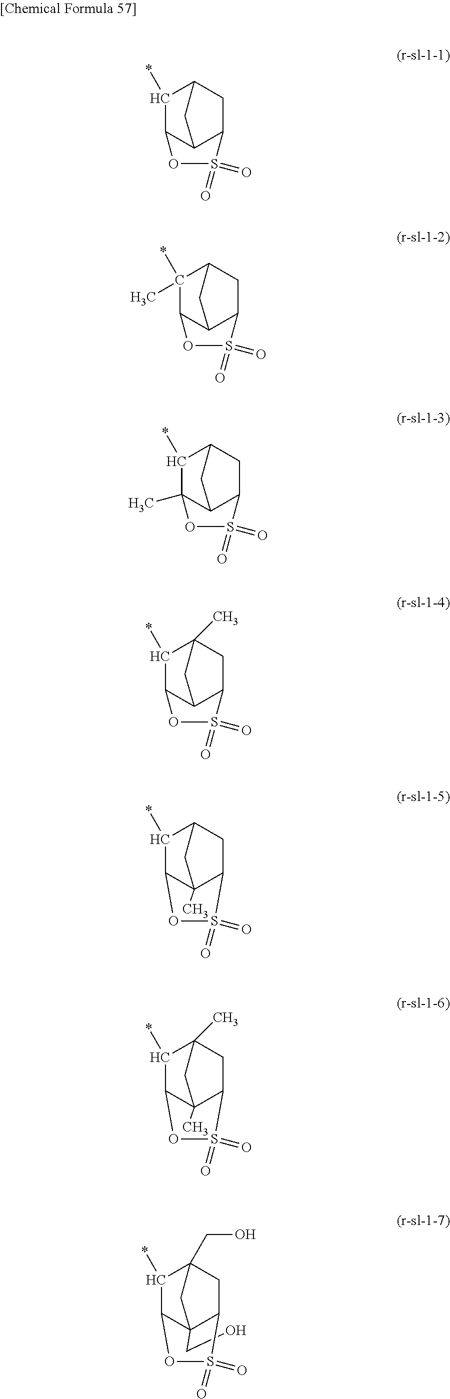

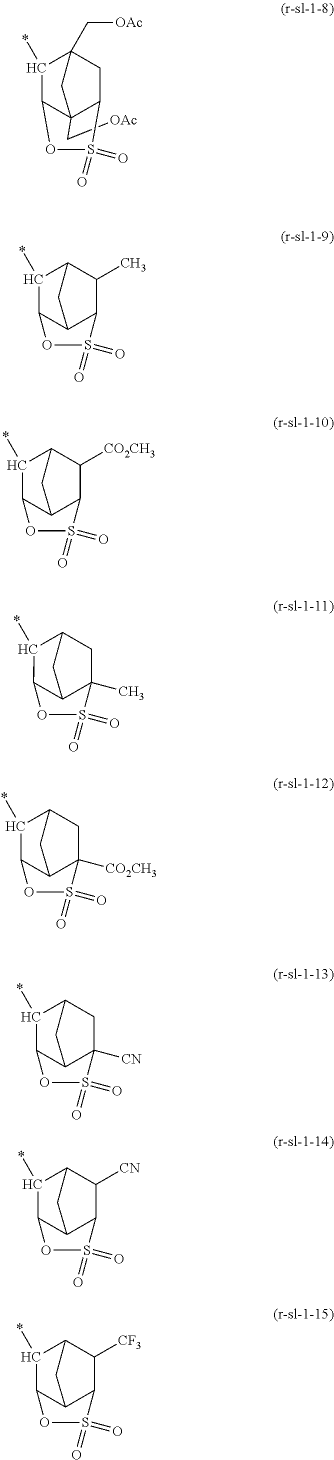

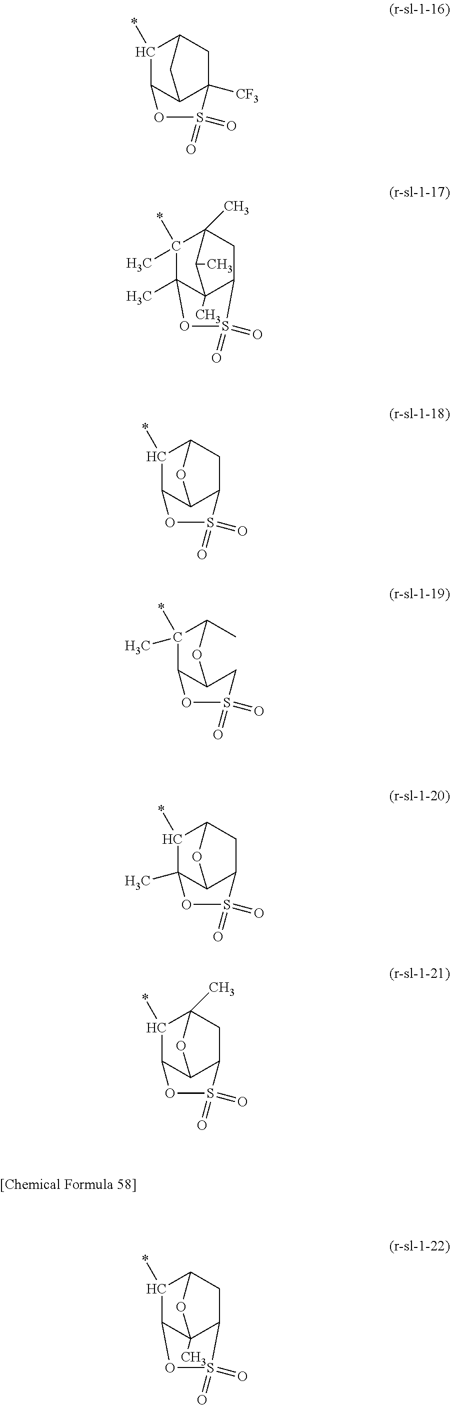

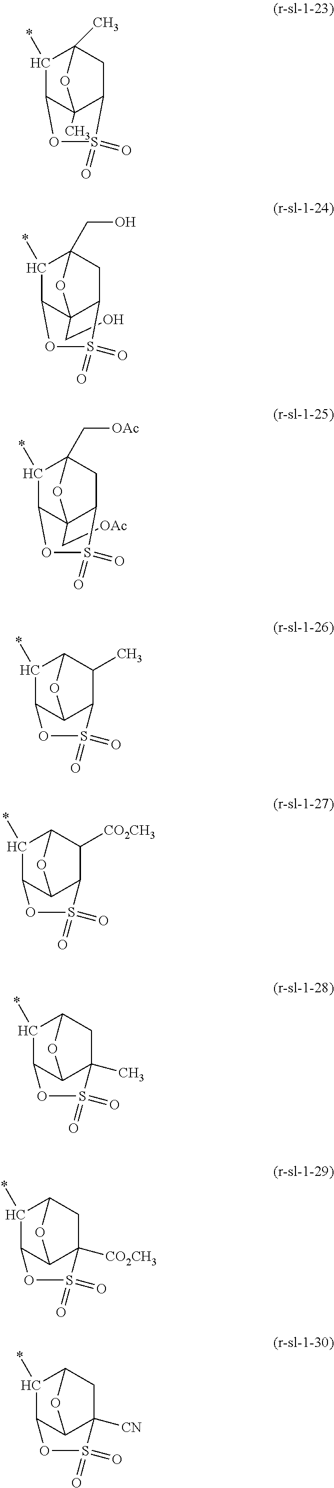

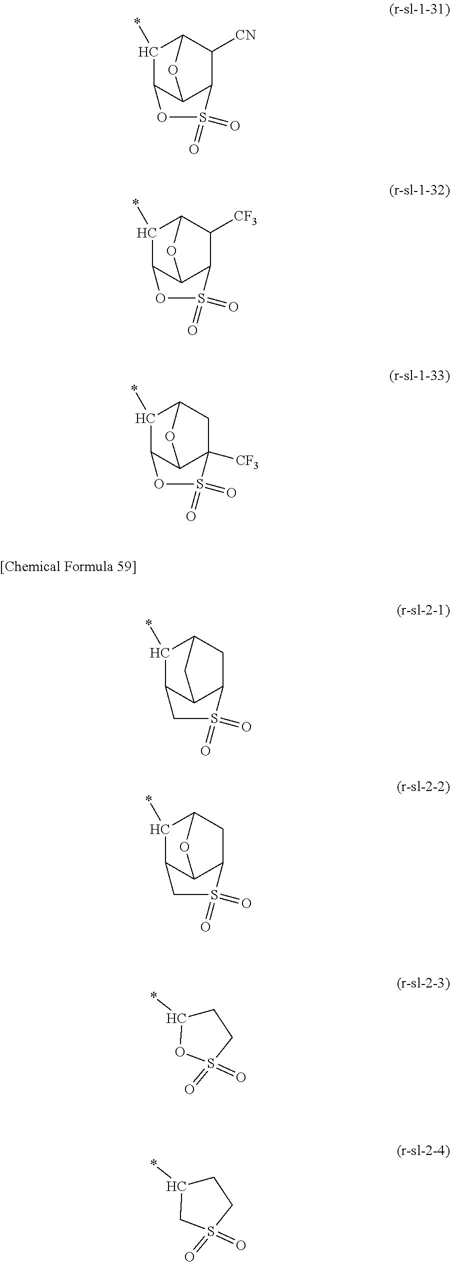

Specific examples of the suitable cation represented by Formula (ca-1) include cations each represented by the following chemical formulae.

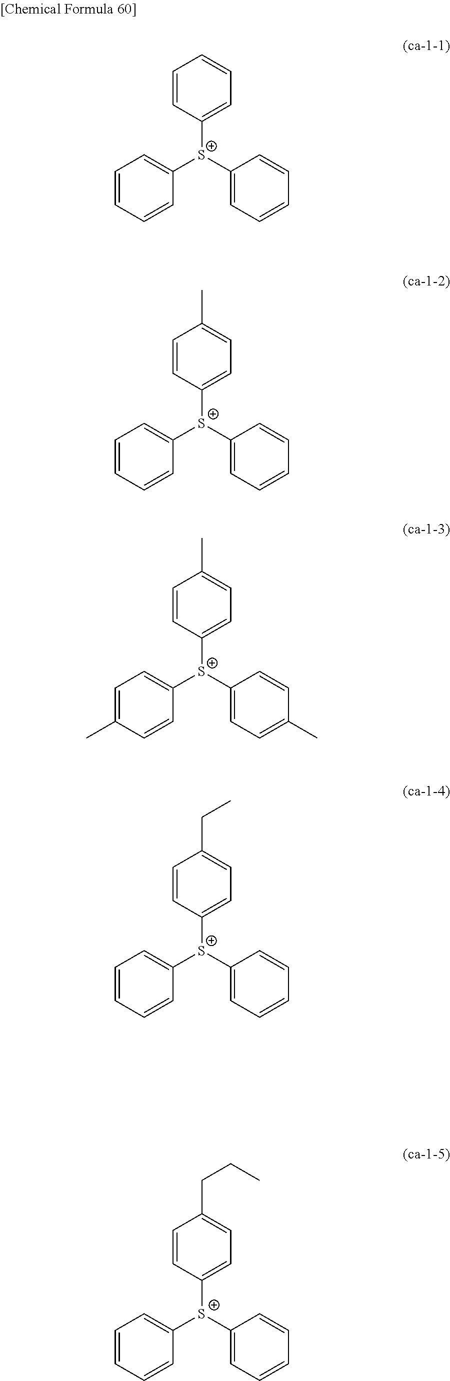

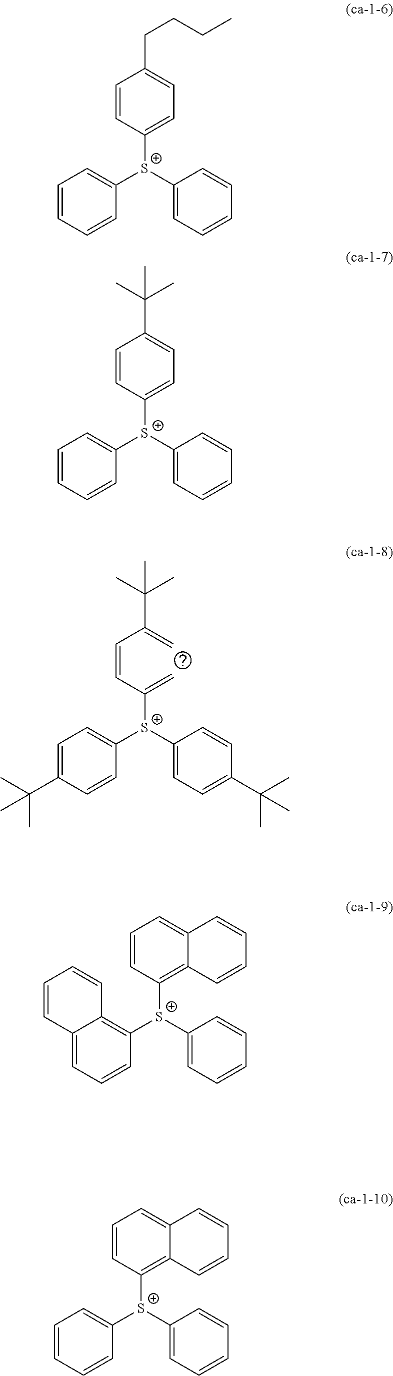 [SMILES: CCCCc1ccc([S+](c2ccccc2)c2ccccc2)cc1 ; CC(C)(C)c1ccc([S+](c2ccccc2)c2ccccc2)cc1 ; C=C(/C=C\C(=C)C(C)(C)C)[S+](c1ccc(C(C)(C)C)cc1)c1ccc(C(C)(C)C)cc1 ; c1ccc([S+](c2cccc3ccccc23)c2cccc3ccccc23)cc1 ; c1ccc([S+](c2ccccc2)c2cccc3ccccc23)cc1]

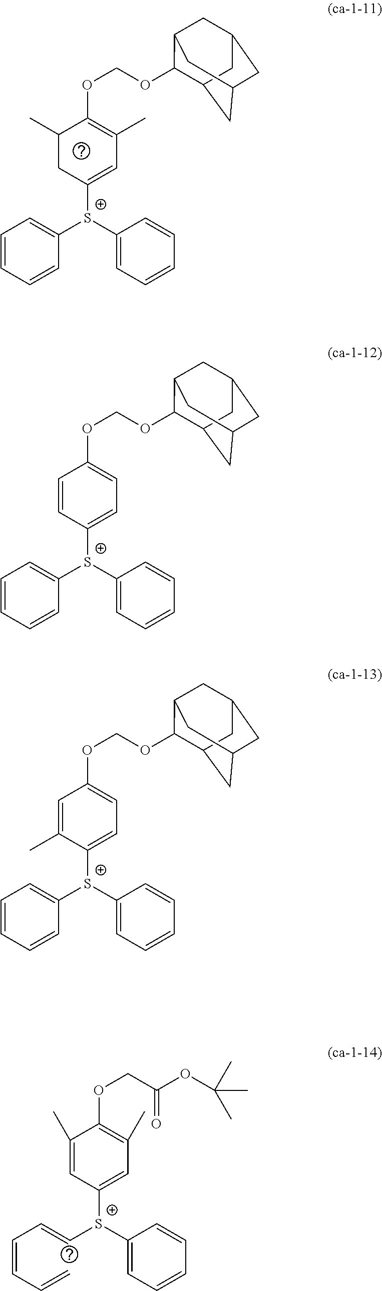 [SMILES: CC1=C(OCOC2C3CC4CC(C3)CC2C4)C(C)CC([S+](c2ccccc2)c2ccccc2)=C1 ; c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; C=C/C=C\C=C\[S+](c1ccccc1)c1cc(C)c(OCC(=O)OC(C)(C)C)c(C)c1]

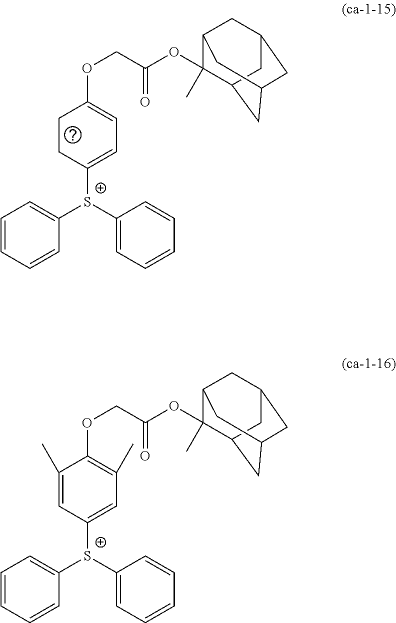 [SMILES: CC1(OC(=O)COC2=CC=C([S+](c3ccccc3)c3ccccc3)CC2)C2CC3CC(C2)CC1C3 ; Cc1cc([S+](c2ccccc2)c2ccccc2)cc(C)c1OCC(=O)OC1(C)C2CC3CC(C2)CC1C3]

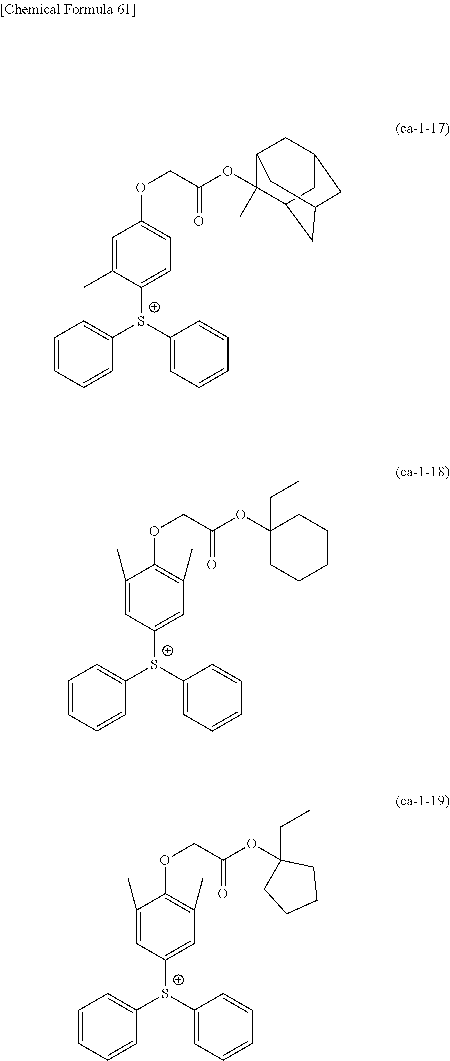 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; CCC1(OC(=O)COc2c(C)cc([S+](c3ccccc3)c3ccccc3)cc2C)CCCC1]

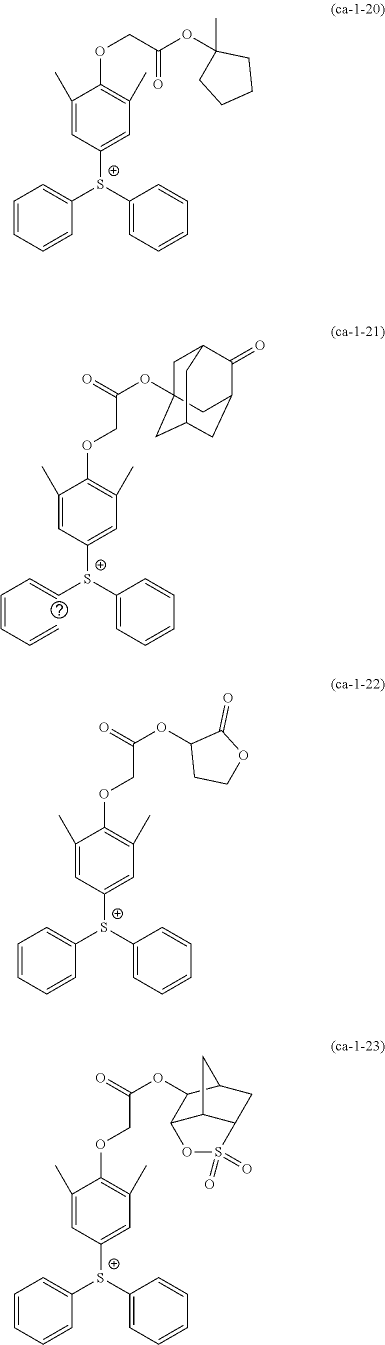 [SMILES: Cc1cc([S+](c2ccccc2)c2ccccc2)cc(C)c1OCC(=O)OC1(C)CCCC1 ; C=C/C=C\C=C\[S+](c1ccccc1)c1cc(C)c(OCC(=O)OC23CC4CC(C2)C(=O)C(C4)C3)c(C)c1 ; Cc1cc([S+](c2ccccc2)c2ccccc2)cc(C)c1OCC(=O)OC1CCOC1=O ; Cc1cc([S+](c2ccccc2)c2ccccc2)cc(C)c1OCC(=O)OC1C2CC3C1OS(=O)(=O)C3C2]

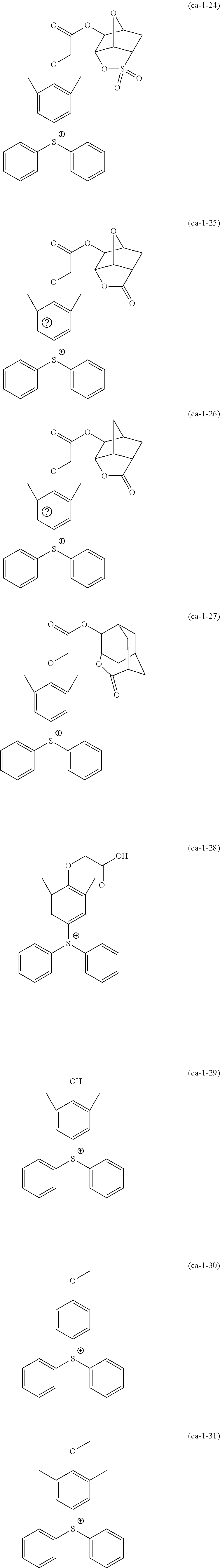

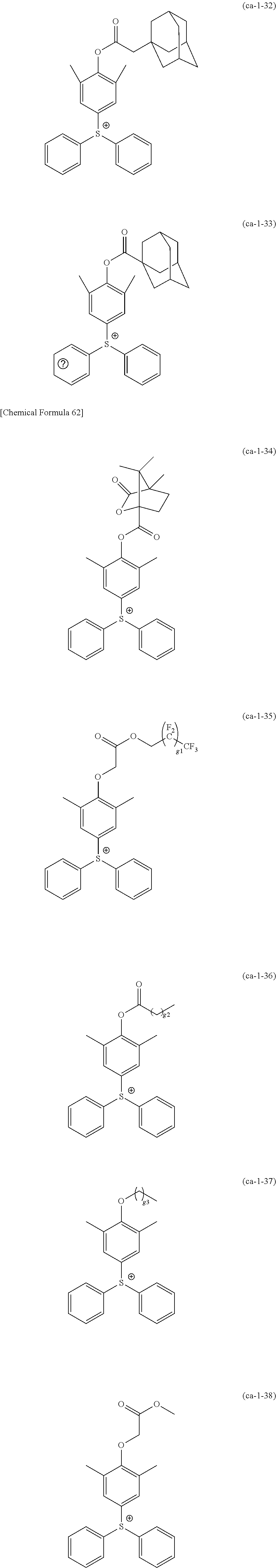

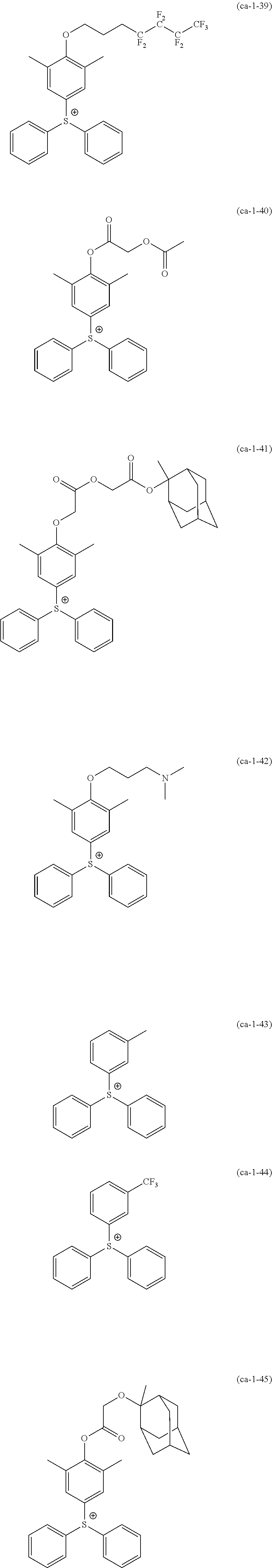

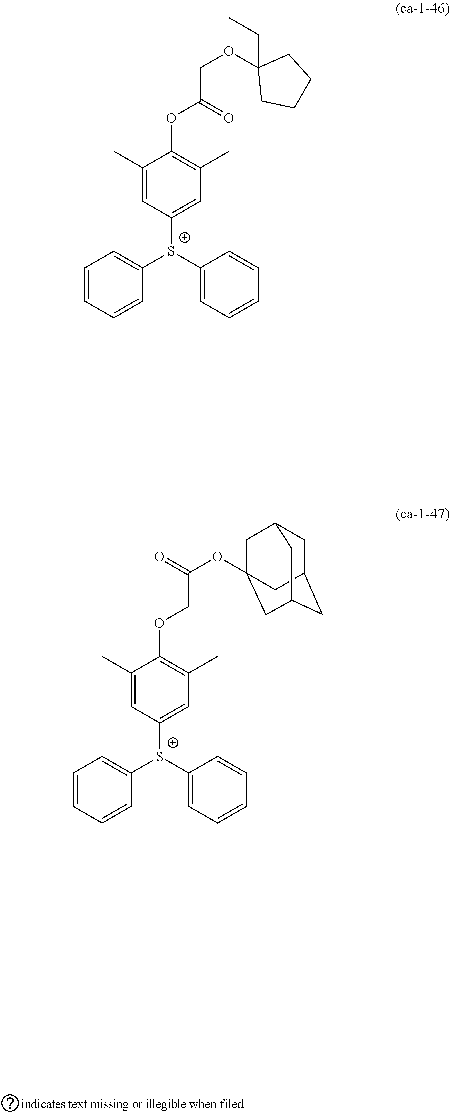 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

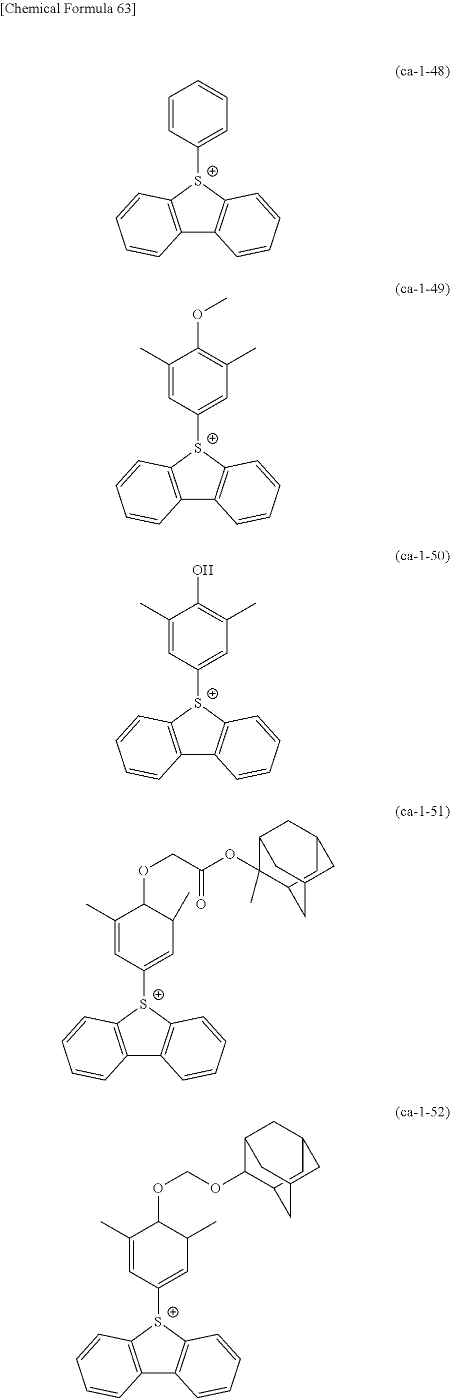 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CC1=CC([s+]2c3ccccc3c3ccccc32)=CC(C)C1OCOC1C2CC3CC(C2)CC1C3]

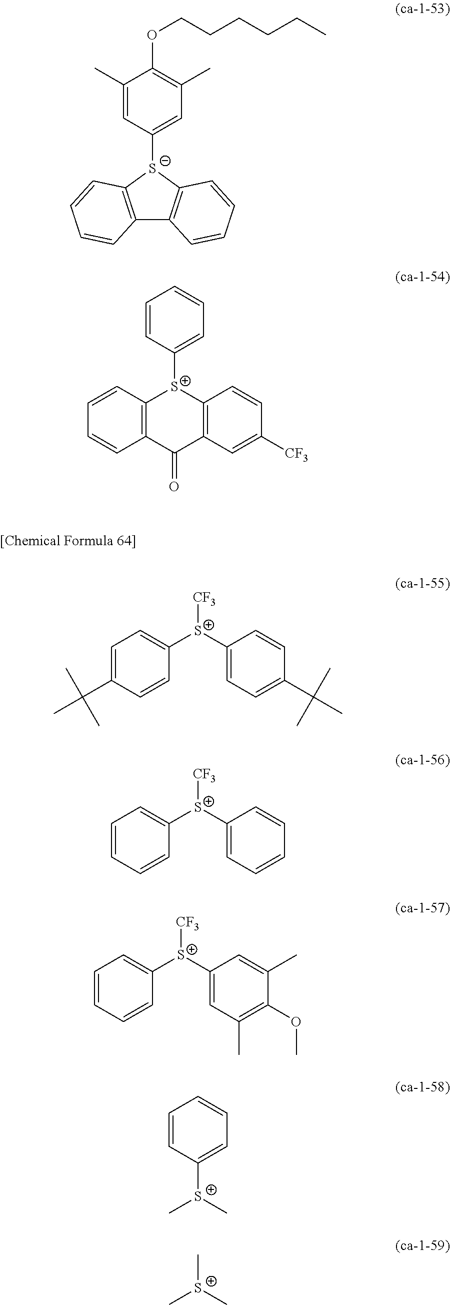

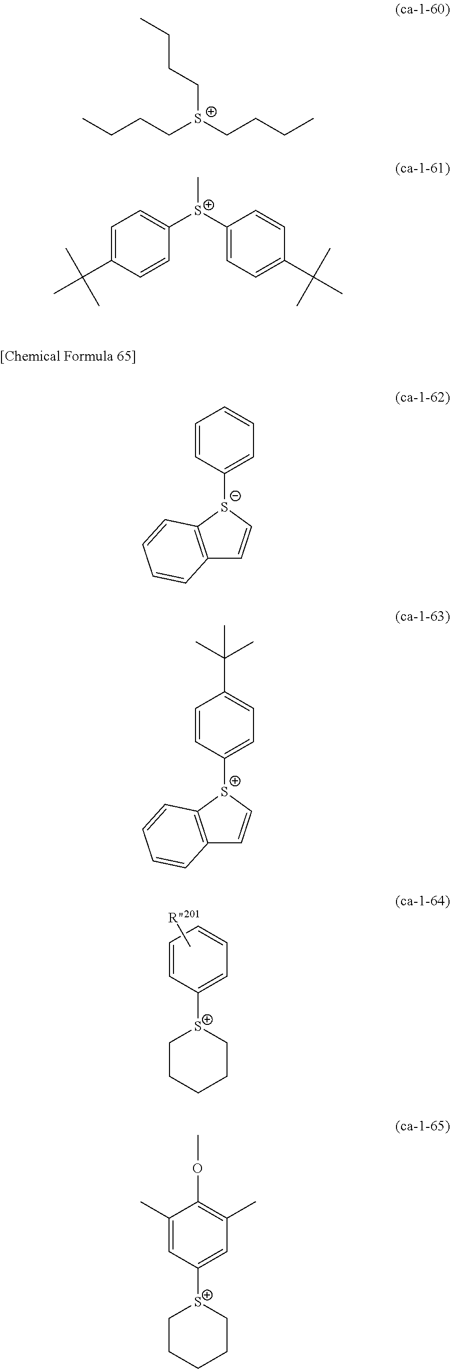

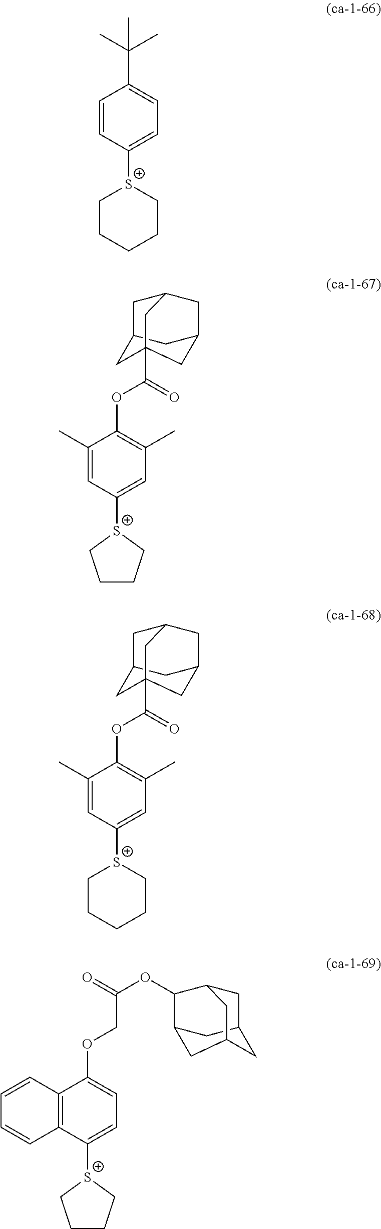 [SMILES: CC(C)(C)c1ccc([S+]2CCCCC2)cc1 ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

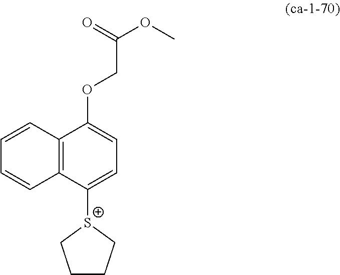 [SMILES: COC(=O)COc1ccc([S+]2CCCC2)c2ccccc12]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

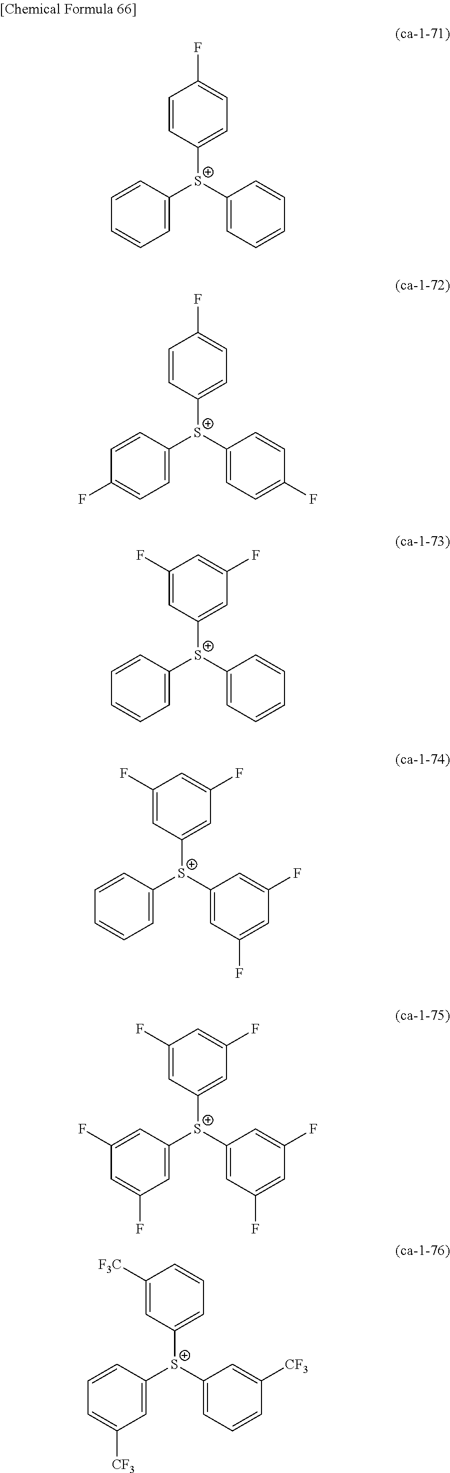 [SMILES: Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1 ; Fc1cc(F)cc([S+](c2ccccc2)c2cc(F)cc(F)c2)c1 ; Fc1cc(F)cc([S+](c2cc(F)cc(F)c2)c2cc(F)cc(F)c2)c1 ; FC(F)(F)c1cccc([S+](c2cccc(C(F)(F)F)c2)c2cccc(C(F)(F)F)c2)c1]

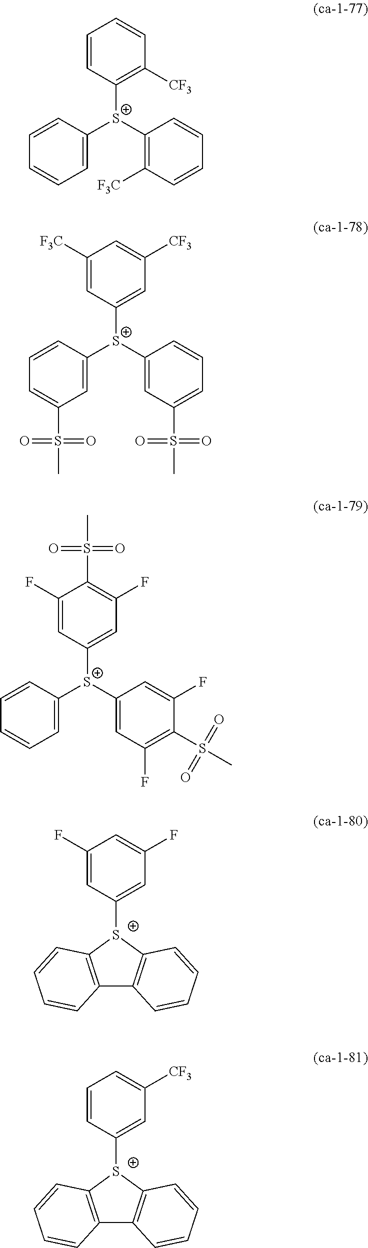 [SMILES: FC(F)(F)c1ccccc1[S+](c1ccccc1)c1ccccc1C(F)(F)F ; CS(=O)(=O)c1cccc([S+](c2cc(C(F)(F)F)cc(C(F)(F)F)c2)c2cccc(S(C)(=O)=O)c2)c1 ; CS(=O)(=O)c1c(F)cc([S+](c2ccccc2)c2cc(F)c(S(C)(=O)=O)c(F)c2)cc1F ; Fc1cc(F)cc(-[s+]2c3ccccc3c3ccccc32)c1 ; FC(F)(F)c1cccc(-[s+]2c3ccccc3c3ccccc32)c1]

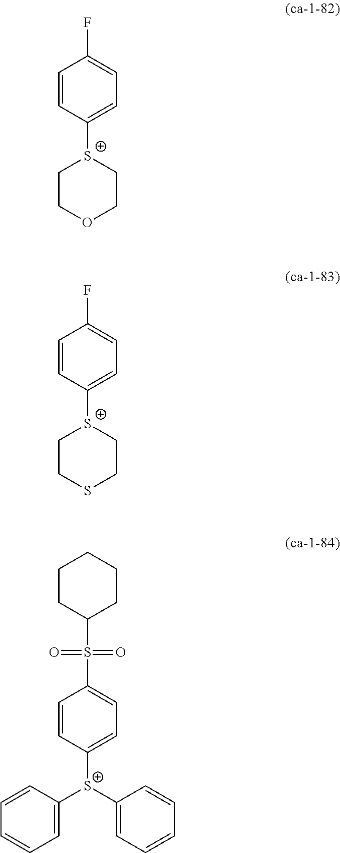 [SMILES: Fc1ccc([S+]2CCOCC2)cc1 ; Fc1ccc([S+]2CCSCC2)cc1 ; O=S(=O)(c1ccc([S+](c2ccccc2)c2ccccc2)cc1)C1CCCCC1]

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

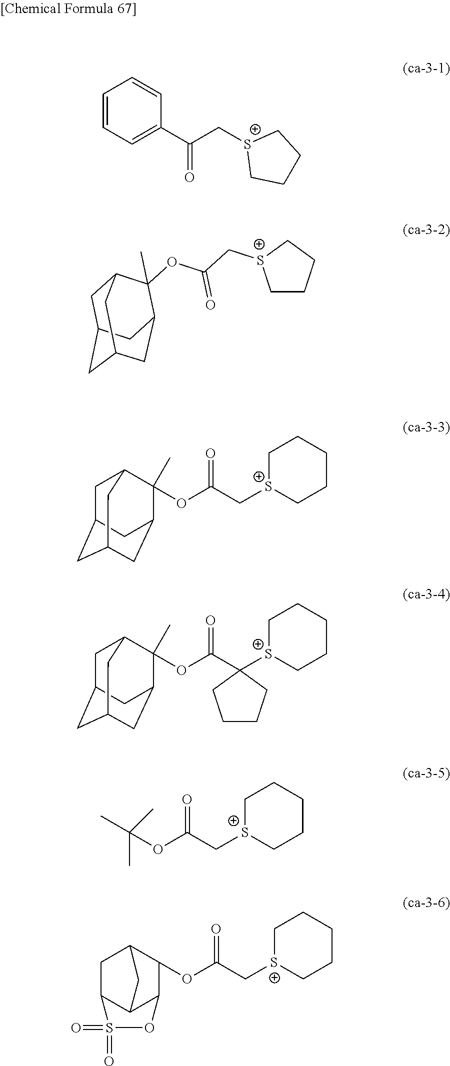 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

As the cation moiety ((M^{m+})_{1/m}), at least one selected from the group consisting of cations each represented by General Formulae (ca-1) to (ca-3) is preferable, and at least one selected from the group consisting of cations each represented by General Formulae (ca-1) and (ca-2) is more preferable. Among these, a cation represented by General Formula (ca-1) is still more preferable.

In particular, from the viewpoint of achieving high sensitivity, the suitable cation represented by Formula (ca-1) preferably has, as a substituent, an electron-withdrawing group such as a fluorine atom, a fluorinated alkyl group, or a sulfonyl group and is, for example, particularly preferably a cation selected from the group consisting of cations each represented by Chemical Formulae (ca-1-44) and (ca-1-71) to (ca-1-84).

Alternatively, the cation moiety ((M^{m+})_{1/m}) is preferably an m-valent sulfonium cation having a fluorine atom, from the viewpoint of achieving high sensitivity. This cation moiety ((M^{m+})_{1/m}) is preferably a cation represented by Formula (ca-1-1).

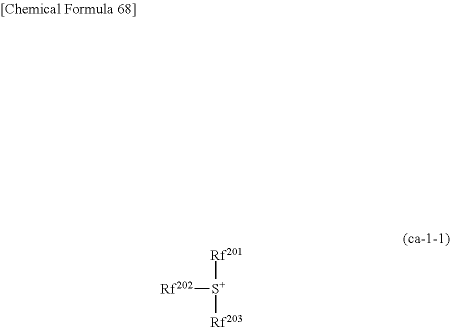

[In the formula, Rf²⁰¹ to Rf²⁰³ each independently represent an aryl group which may have a substituent, an alkyl group which may have a substituent, or an alkenyl group which may have a substituent. Rf²⁰¹ to Rf²⁰³ may be bonded to each other to form a ring together with the sulfur atoms in the formula. Here, at least one of Rf²⁰¹ to Rf²⁰³ contains at least one fluorine atom.]

Rf²⁰¹ to Rf²⁰³ in Formula (ca-1-1) are each have the same as that for R²⁰¹ to R²⁰³ in Formula (ca-1). Here, at least one of Rf²⁰¹ to Rf²⁰³ contains at least one fluorine atom. The cation represented by Formula (ca-1-1) preferably contains three or more fluorine atoms. Any one of Rf²⁰¹ to Rf²⁰³ may have three or more fluorine atoms, or the total number of fluorine atoms in Rf²⁰¹ to Rf²⁰³ may be three or more.

Specific examples of the component (D0) are shown below but are not limited thereto.

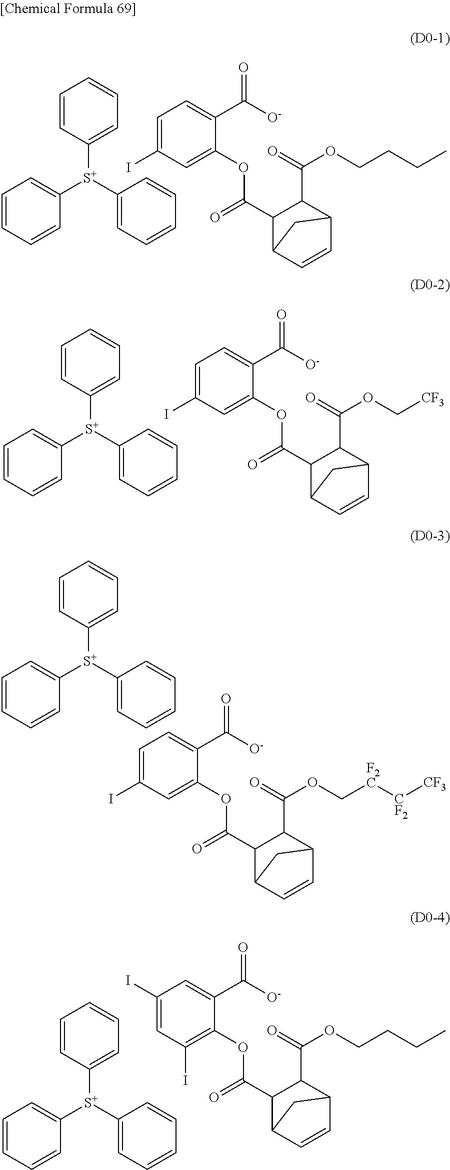

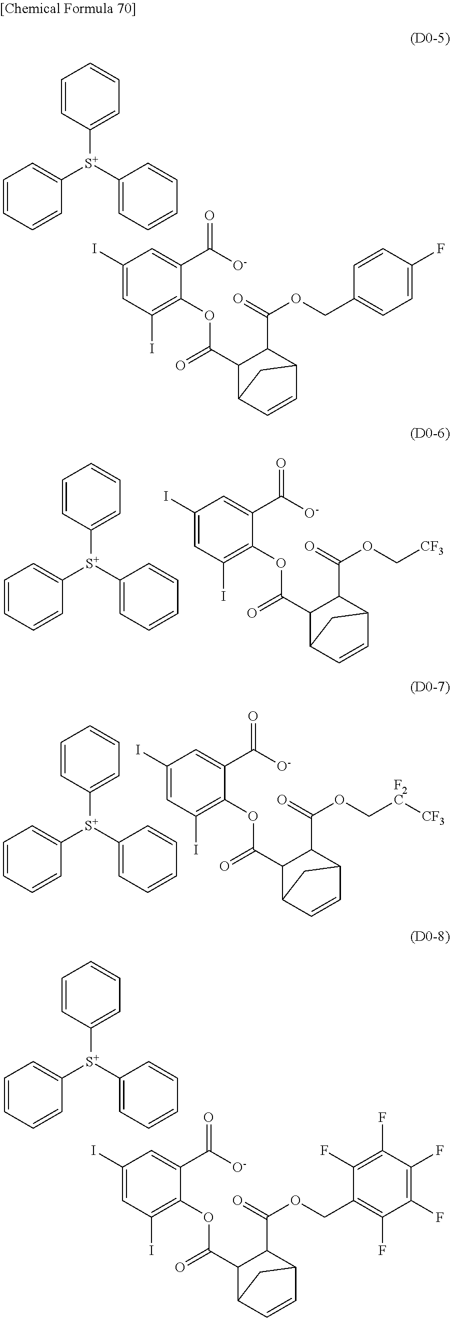

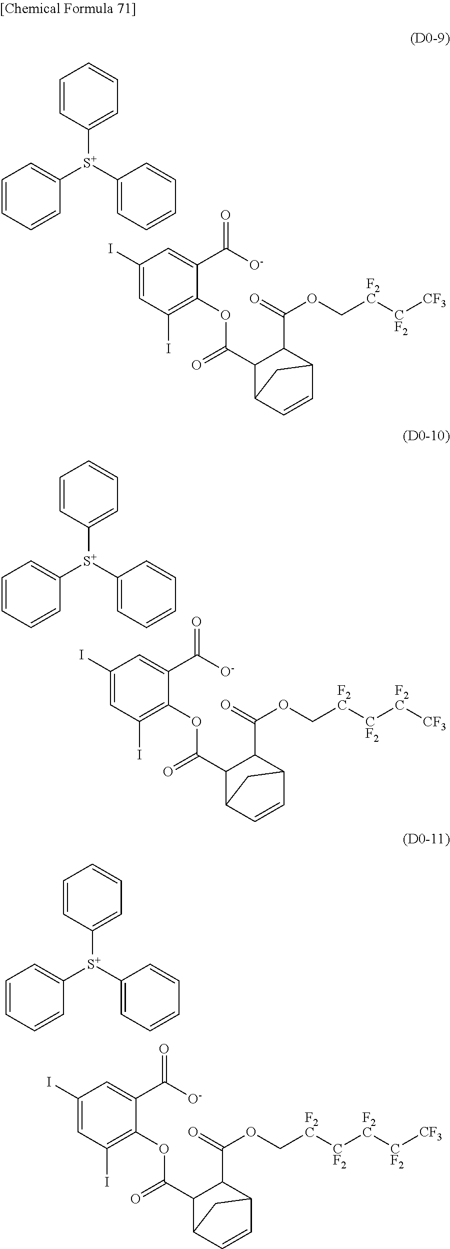 [SMILES: c1ccc([S+](c2ccccc2)c2ccccc2)cc1 ; O=C([O-])c1cc(I)cc(I)c1OC(=O)C1C2C=CC(C2)C1C(=O)OCC(F)(F)C(F)(F)C(F)(F)F ; c1ccc([S+](c2ccccc2)c2ccccc2)cc1 ; O=C([O-])c1cc(I)cc(I)c1OC(=O)C1C2C=CC(C2)C1C(=O)OCC(F)(F)C(F)(F)C(F)(F)C(F)(F)F ; c1ccc([S+](c2ccccc2)c2ccccc2)cc1 ; O=C([O-])c1cc(I)cc(I)c1OC(=O)C1C2C=CC(C2)C1C(=O)OCC(F)(F)C(F)(F)C(F)(F)C(F)(F)C(F)(F)F]

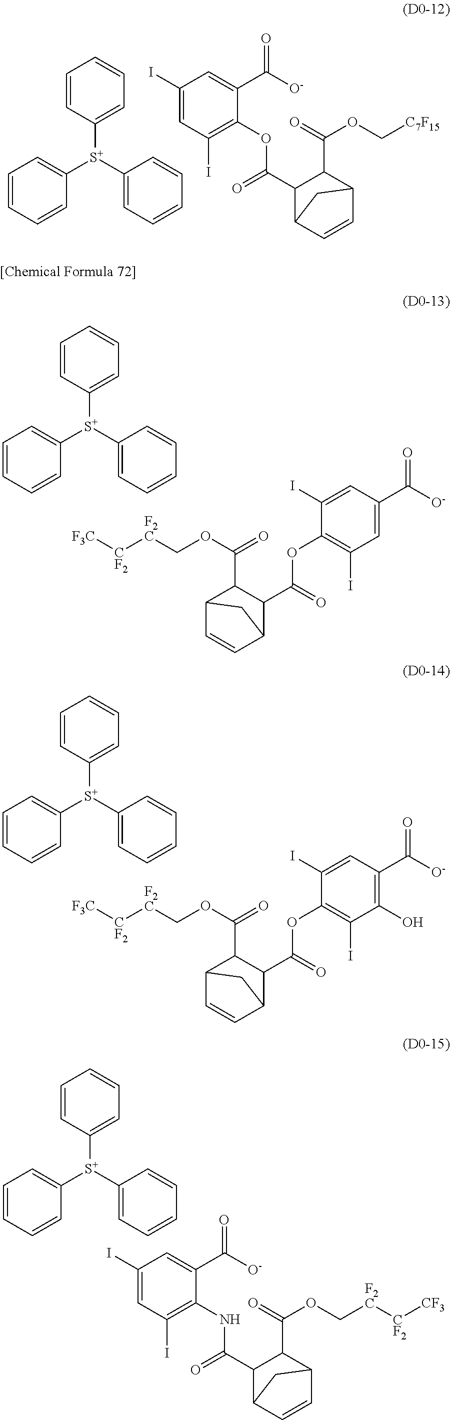

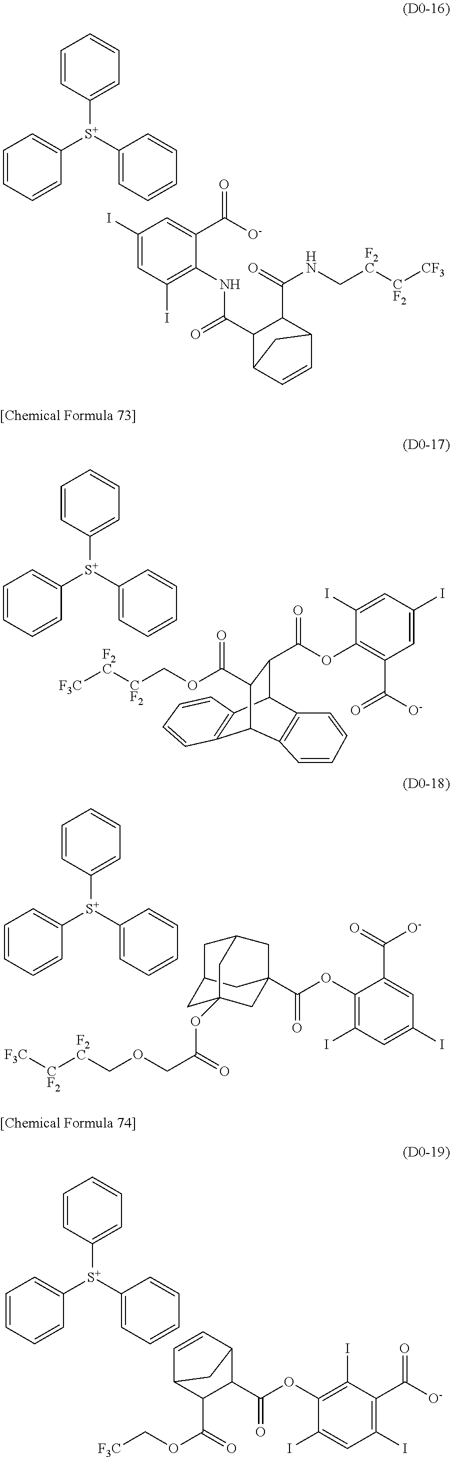

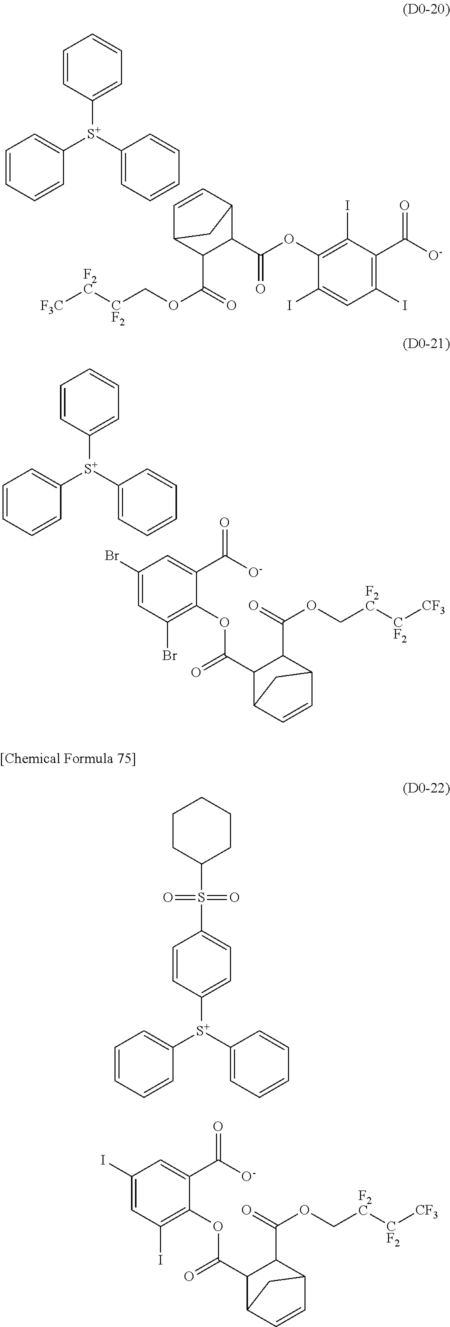 [SMILES: c1ccc([S+](c2ccccc2)c2ccccc2)cc1 ; O=C([O-])c1c(I)cc(I)c(OC(=O)C2C3C=CC(C3)C2C(=O)OCC(F)(F)C(F)(F)C(F)(F)F)c1I ; c1ccc([S+](c2ccccc2)c2ccccc2)cc1 ; O=C([O-])c1cc(Br)cc(Br)c1OC(=O)C1C2C=CC(C2)C1C(=O)OCC(F)(F)C(F)(F)C(F)(F)F ; O=S(=O)(c1ccc([S+](c2ccccc2)c2ccccc2)cc1)C1CCCCC1 ; O=C([O-])c1cc(I)cc(I)c1OC(=O)C1C2C=CC(C2)C1C(=O)OCC(F)(F)C(F)(F)C(F)(F)F]

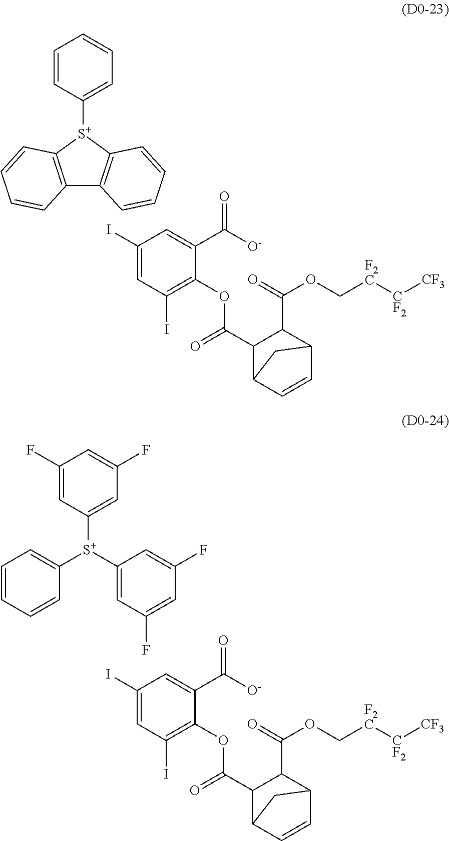 [SMILES: c1ccc(-[s+]2c3ccccc3c3ccccc32)cc1 ; O=C([O-])c1cc(I)cc(I)c1OC(=O)C1C2C=CC(C2)C1C(=O)OCC(F)(F)C(F)(F)C(F)(F)F ; Fc1cc(F)cc([S+](c2ccccc2)c2cc(F)cc(F)c2)c1 ; O=C([O-])c1cc(I)cc(I)c1OC(=O)C1C2C=CC(C2)C1C(=O)OCC(F)(F)C(F)(F)C(F)(F)F]

Among the examples, the component (D0) is preferably a component selected from the group consisting of compounds each represented by Chemical Formulae (D0-1) to (D0-24).

Alternatively, among the example, from the viewpoints of roughness reduction and the pattern shape (rectangularity), the component (D0) is preferably a compound having an anion in which the total number of fluorine atoms contained in Rf in General Formula (d0) is 5 or more and 9 or less and more preferably a compound selected from the group consisting of compounds each represented by Chemical Formulae (D0-3), (D0-7), (D0-9), (D0-10), (D0-13), (D0-14), (D0-17), (D0-18), and (D0-20) to (D0-24).

Alternatively, among the examples, from the viewpoint of achieving high sensitivity, the component (D0) is preferably a compound having an anion in which R^{d0} in General Formula (d0) represents an iodine atom and y represents 2 or 3 and more preferably a compound selected from the group consisting of compounds each represented by Chemical Formulae (D0-4) to (D0-20) and (D0-22) to (D0-24).

Alternatively, among the examples, from the viewpoints of high sensitivity, roughness reduction, and the pattern shape (rectangularity), the component (D0) is particularly preferably a compound selected from the group consisting of compounds each represented by Chemical Formulae (D0-7), (D0-9), (D0-10), (D0-13), (D0-14), (D0-17), (D0-18), (D0-20), and (D0-22) to (D0-24).

In the resist composition according to the present embodiment, the component (D0) may be used alone or may be used in a combination of two or more kinds thereof.

In the resist composition according to the present embodiment, the content of the component (D0) is preferably in a range of 0.5 to 20 parts by mass, more preferably in a range of 1 to 15 parts by mass, still more preferably in a range of 2 to 15 parts by mass, and particularly preferably in a range of 3 to 12.5 parts by mass with respect to 100 parts by mass of the component (A).

In a case where the content of the component (D0) is greater than or equal to the lower limits of the above-described preferable ranges, high sensitivity can be achieved and lithography characteristics such as rectangularity of the pattern shape and roughness reduction are likely to be enhanced in the resist pattern formation. Meanwhile, in a case where the content thereof is less than or equal to the upper limits of the above-described preferable ranges, the sensitivity can be maintained more satisfactorily. Further, it is preferable that each component of the resist composition is dissolved in an organic solvent from the viewpoint that a uniform solution is easily obtained and the storage stability of the resist composition is enhanced.

The proportion of the component (D0) in the entire component (D) is, for example, 50% by mass or greater, preferably 70% by mass or greater, and more preferably 95% by mass or greater. The proportion of the component (D0) in the total amount of the component (D) may be 100% by mass.

The component (D) in the resist composition according to the present embodiment may contain a base component other than the above-described component (D0).

Examples of the base component other than component (D0) include a photodecomposable base (D1) having acid diffusion controllability (hereinafter, referred to as “component (D1)”) which is lost by decomposition upon light exposure and a nitrogen-containing organic compound (D2) (hereinafter, referred to as “component (D2)”) which does not correspond to the component (D1). Among these, a photodecomposable base (component (D1)) is preferable from the viewpoint that all the characteristics of high sensitivity, in-plane uniformity (CDU) of pattern dimensions, fine resolution, and suppression of occurrence of coating defects are likely to be enhanced. The compound described as the component (D1) below may be used as the acid generator component (component (B)) depending on the combination with other compounds.

### In Regard to Component (D1)

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not act as a quencher at the exposed portion of the resist film, but act as a quencher at the unexposed portion of the resist film.

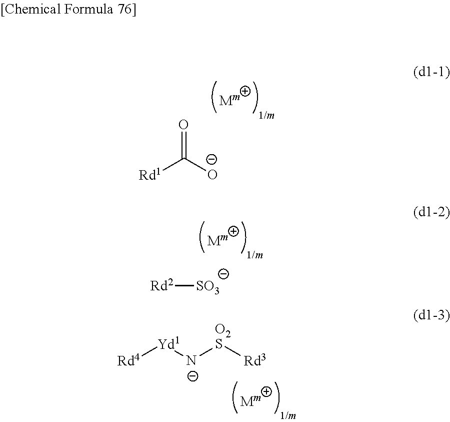

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and M^{m+}s each independently represents an m-valent cation.]
   {Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples of each of the cyclic groups include the same groups as those for R′²⁰¹. Among these, it is preferable that the group as Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-8), an ether bond, an ester bond, and a combination thereof. In a case where an ether bond or an ester bond is included as the substituent, the substituent may be bonded through an alkylene group, and a linking group represented by any of Formulae (y-a1-1) to (y-a1-5) is preferable as the substituent. Further, in a case where the aromatic hydrocarbon group, the aliphatic cyclic group, or the chain-like alkyl group as Rd¹ contains a linking group represented by any of General Formulae (y-a1-1) to (y-a1-7) as a substituent, V″⁰¹ in General Formula (y-a1-1) to (y-a1-7) is bonded to the carbon atom constituting the aromatic hydrocarbon group, the aliphatic cyclic group, or the chain-like alkyl group as Rd¹ in General Formula (d3-1), in Formulae (y-a1-1) to (y-a1-7).

Suitable examples of the aromatic hydrocarbon group include a phenyl group, a naphthyl group, and a polycyclic structure having a bicyclooctane skeleton (a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

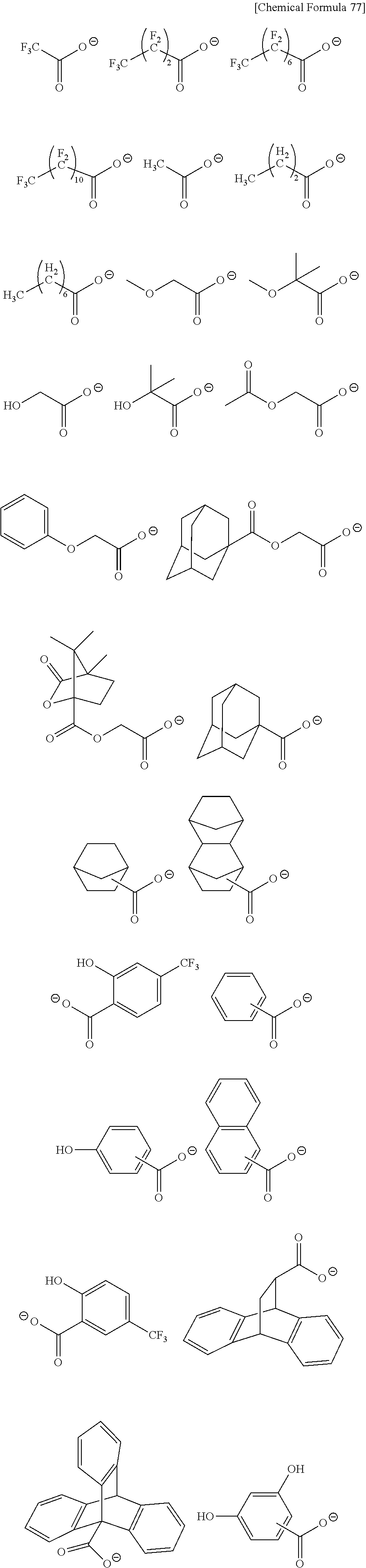

### Cation Moiety

In Formula (d1-1), M^{m+} represents an m-valent cation.

Suitable examples of the cation as M^{m+} include the same cations as those for the cations each represented by General Formulae (ca-1) to (ca-3). Among these, a cation represented by General Formula (ca-1) is more preferable, and cations each represented by Formulae (ca-1-1) to (ca-1-84) are still more preferable.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with fluorine). As a result, the anion of the component (d1-2) becomes an appropriately weak acid anion, thereby improving the quenching ability of the component (D).

Rd² represents preferably a chain-like alkyl group which may have a substituent or an aliphatic cyclic group which may have a substituent, and more preferably an aliphatic cyclic group which may have a substituent.

The chain-like alkyl group has preferably 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same substituents as those that the hydrocarbon group (an aromatic hydrocarbon group, an aliphatic cyclic group, or a chain-like alkyl group) as Rd¹ in Formula (d1-1) may have.

Specific preferred examples of the anion moiety in the component (d1-2) are described below.

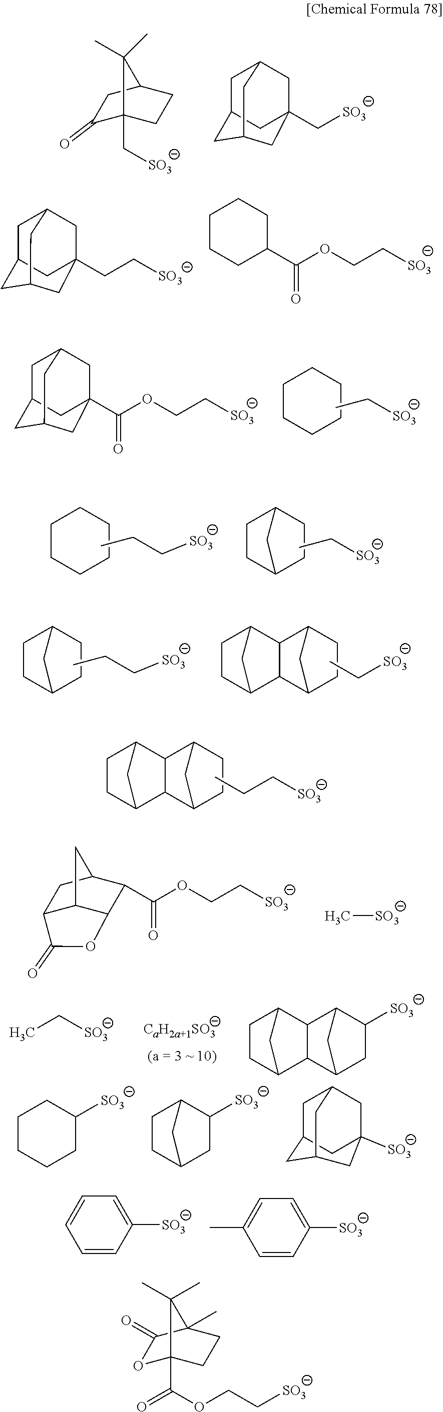

### Cation Moiety

In Formula (d1-2), M^{m+} represents an m-valent cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may further contain an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group. The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

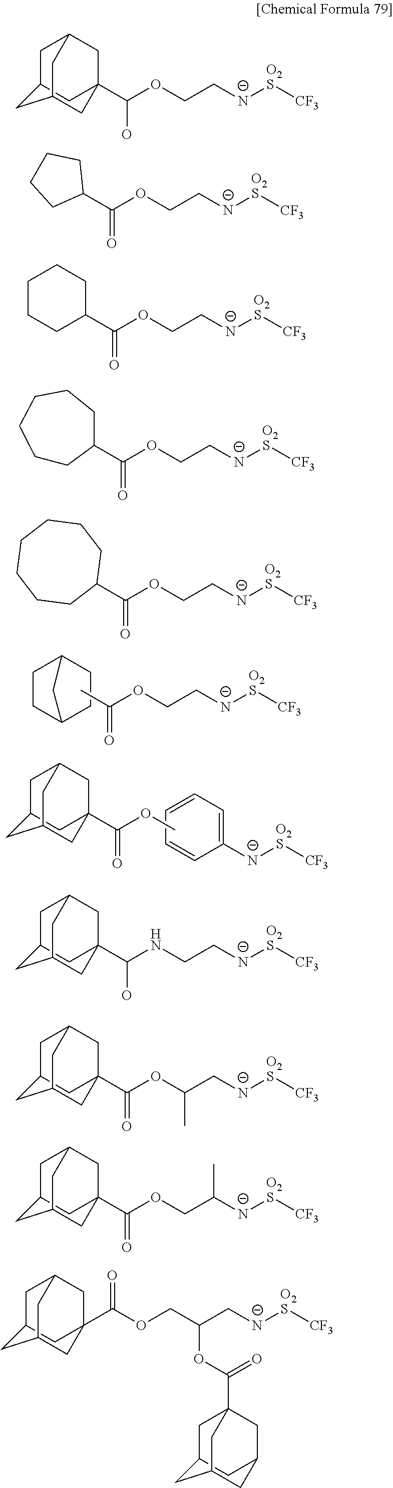

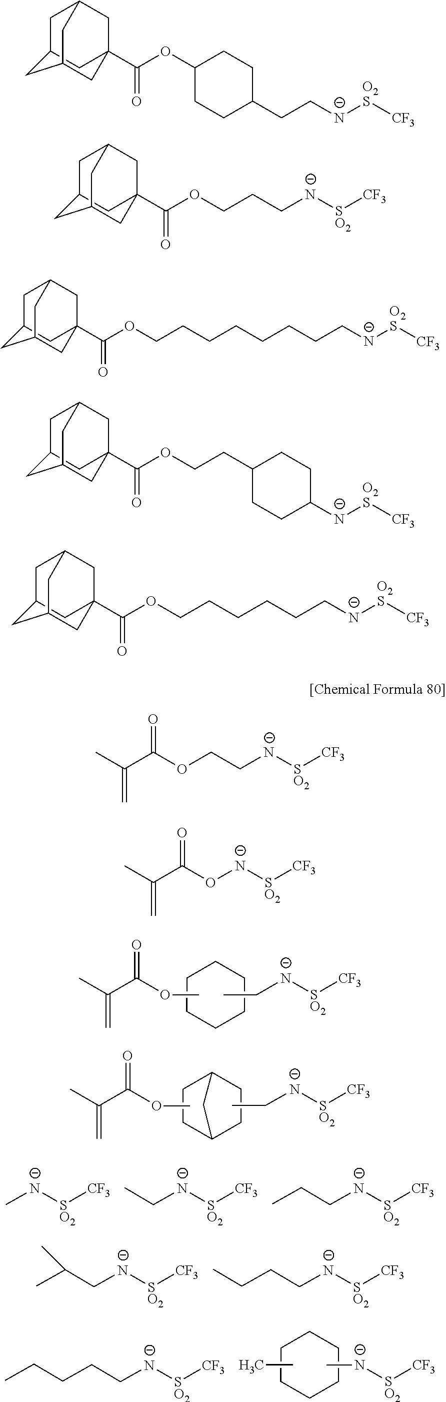

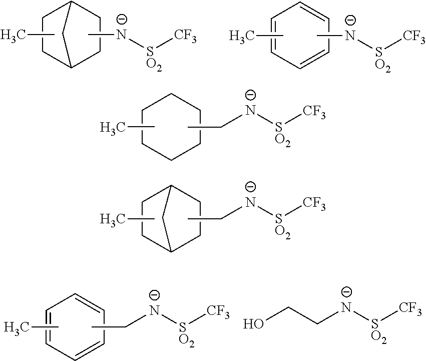

### Cation Moiety

In Formula (d1-3), M^{m+} represents an m-valent cation and has the same definition as that for M^{m+} in Formula (d1-1).

The component (d1-3) may be used alone or in combination of two or more kinds thereof.

As the component (D1), only any one of the above-described components (d1-1) to (d1-3) or a combination of two or more kinds thereof may be used.

In a case where the resist composition contains the component (D1), the content of the component (D1) in the resist composition is preferably in a range of 0.5 to 15 parts by mass, more preferably in a range of 1 to 12 parts by mass, and still more preferably in a range of 2 to 10 parts by mass with respect to 100 parts by mass of the component (A).

It is preferable that the component (D1) contains the component (d1-1).

The content of the component (d1-1) in the total component (D1) is preferably 50% by mass or more, preferably 70% by mass or more, and still more preferably 90% by mass or more, and the component (D1) may consist of only a compound for the component (d1-1).

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as a “component (D2)”) which does not correspond to the component (D1).

The component (D2) is not particularly limited as long as it acts as an acid diffusion control agent and does not correspond to the component (D1), and any known compound may be used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

It is preferable that the aliphatic polycyclic amine has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of the aromatic amine include 4-dimethylaminopyridine, pyrrole, indole, pyrazole, imidazole, and derivatives thereof, tribenzylamine, 2,6-diisopropylaniline, N-tert-butoxycarbonylpyrrolidine, 2,6-di-tert-butylpyridine, and 2,6-tert-butylpyridine.

The component (D2) may be used alone or in combination of two or more kinds thereof.

In a case where the resist composition contains the component (D2), the content of the component (D2) in the resist composition is typically in a range of 0.01 to 5 parts by mass with respect to 100 parts by mass of the component (A). In a case where the content thereof is set to be in the above-described range, the resist pattern shape, the post-exposure temporal stability, and the like are improved.

### <Other Components>

The resist composition according to the present embodiment may further contain other components in addition to the component (A) and the component (D) described above. Examples of the other components include a component (B), a component (E), a component (F), and a component (S), which are described below.

### <<Component (B): Acid Generator Component>>

It is preferable that the resist composition of the present embodiment further contains an acid generator component (B) (here, the compound (D0) is excluded) that generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

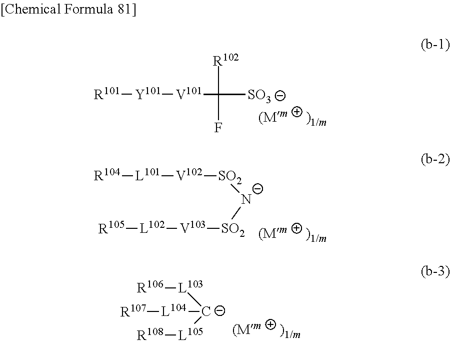

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. Here, both Y¹⁰¹ and V¹⁰¹ do not form a single bond. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring-based polycyclic skeleton such as adamantane, norbornane, isobornane, tricyclodecane, or tetracyclododecane; and a polycycloalkane having a condensed ring-based polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site with respect to Y¹⁰¹ in Formula (b-1).

Examples of the substituent for the cyclic group as R¹⁰¹ include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

An alkoxy group having 1 to 5 carbon atoms is preferable as the alkoxy group serving as the substituent.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰ may be a condensed cyclic group containing a condensed ring in which an aliphatic hydrocarbon ring and an aromatic ring are condensed. Examples of the condensed ring include those obtained by condensing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site with respect to Y¹⁰¹ in Formula (b-1).

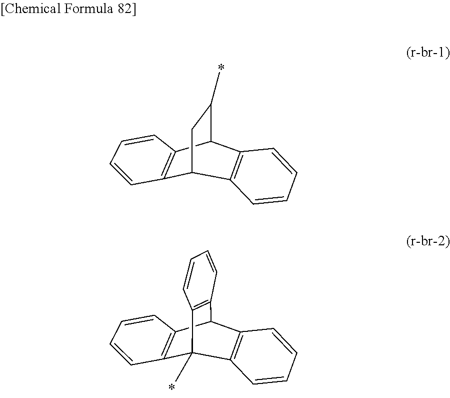

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include the same groups as those for the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-Like Alkyl Group which May have Substituent:

The chain-like alkyl group as R¹⁰ may be linear or branched.

The number of carbon atoms in the linear alkyl group is preferably in a range of 1 to 20, more preferably in a range of 1 to 15, and most preferably in a range of 1 to 10.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹.

In Formula (b-1), Y¹⁰¹ represents a single bond or a divalent linking group having an oxygen atom.

In a case where Y¹⁰1 represents a divalent linking group having an oxygen atom, Y¹⁰¹ may have an atom other than the oxygen atom. Examples of atoms other than an oxygen atom include a carbon atom, a hydrogen atom, a sulfur atom, and a nitrogen atom.

Examples of the divalent linking group having an oxygen atom include linking groups each represented by General Formulae (y-a1-1) to (y-a1-8).

In Formula (b-1), V¹⁰¹ represents a single bond, an alkylene group, or a fluorinated alkylene group. It is preferable that the alkylene group and the fluorinated alkylene group as V¹⁰¹ have 1 to 4 carbon atoms. Among these, it is preferable that V¹⁰¹ represents a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰1 represents a single bond, specific examples of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

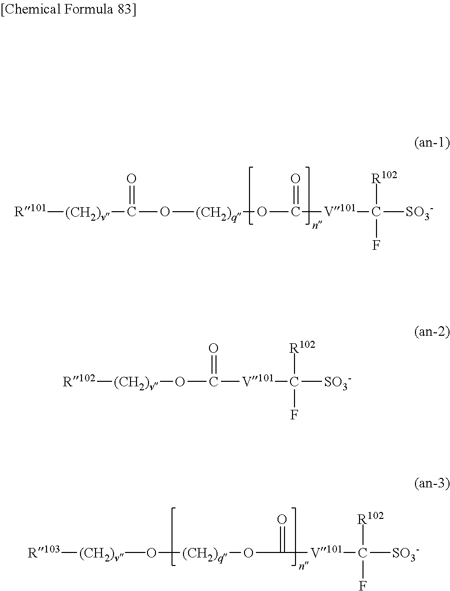

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R′¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group which may have a substituent as R″¹⁰¹ and R″¹⁰³, the groups described as the aromatic hydrocarbon group in the cyclic hydrocarbon group as R¹⁰¹ in Formula (b-1) are preferable. Examples of the substituent include the same substituents as those which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light with a wavelength of 250 nm or less or electron beams is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples of each of the groups include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include those for R¹⁰¹ in Formula (b-1).

In Formula (b-3), L¹⁰³ to L¹⁰⁵ each independently represent a single bond, —CO—, or —SO₂—.

Among the examples, as the anion moiety of the component (B), an anion in the component (b-1) is preferable.

### {Cation Moiety}

In Formulae (b-1), (b-2), and (b-3), M′^{m+} represents an m-valent onium cation. Among them, a sulfonium cation and an iodonium cation are preferable.

m represents an integer of 1 or greater.

Preferred examples of the cation moiety ((M′^{m+})_{1/m}) include the same cations as those for the cations each represented by General Formulae (ca-1) to (ca-3). Among these, a cation represented by General Formula (ca-1) is more preferable, and cations each represented by Formulae (ca-1-1) to (ca-1-84) are still more preferable.

In the resist composition according to the present embodiment, the component (B) may be used alone or in combination of two or more kinds thereof.

In a case where the resist composition contains the component (B), the content of the component (B) in the resist composition is preferably less than 50 parts by mass, more preferably in a range of 5 to 40 parts by mass, and still more preferably in a range of 10 to 40 parts by mass with respect to 100 parts by mass of the component (A).

In a case where the content of the component (B) is set to be in the above-described preferable ranges, pattern formation can be sufficiently carried out. Further, it is preferable that each component of the resist composition is dissolved in an organic solvent from the viewpoint that a uniform solution is easily obtained and the storage stability of the resist composition is enhanced.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape, the post-exposure temporal stability, and the like, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). Within the above range, the lithography characteristics are further improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

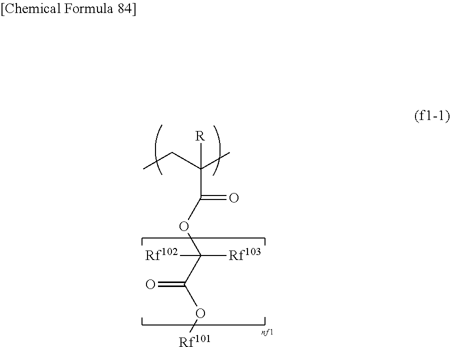

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R.

Among the examples, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and particularly preferably 60% or greater thereof are fluorinated from the viewpoint of increasing the hydrophobicity of the resist film during immersion exposure.

Among examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is satisfactory.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition according to the present embodiment may be produced by dissolving the resist materials in an organic solvent component (hereinafter, referred to as a “component (S)”).

In the resist composition of the present embodiment, the component (S) may be used alone or in the form of a mixed solvent of two or more kinds thereof. Among these, propylene glycol monomethyl ether acetate (PGMEA), propylene glycol monomethyl ether (PGME), γ-butyrolactone, ethyl lactate (EL), or cyclohexanone is preferable.

Further, a mixed solvent obtained by mixing PGMEA with a polar solvent is also preferable as the component (S). The blending ratio (mass ratio) may be appropriately determined in consideration of the compatibility or the like between PGMEA and the polar solvent.

A mixed solvent of at least one selected from PGMEA and EL, and γ-butyrolactone is also preferable as the (S) component. In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

After the resist material is dissolved in the component (S), impurities and the like may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition of the present embodiment described above contains the base material component (A) and the compound (D0) represented by General Formula (d0) (component (D0).

The component (D0) generates a carboxylic acid upon light exposure and can act as a quencher (acid diffusion control agent) in an unexposed portion of the resist film. In the component (D0), a hydrocarbon group (Rf) which may have a fluorine atom is bonded to a benzene ring of the anion moiety via an iodine atom or a bromine atom (R^{d0}) and a linking group (-L¹-L²-L³-).

The iodine atom or the bromine atom (R^{d0}) has high hydrophobicity and large absorption of EUV with a wavelength of 13.5 nm. In the present embodiment, since the anion moiety of the component (D0) has an iodine atom or a bromine atom (R^{d0}), the hydrophobicity of the unexposed portion of the resist film is increased, and the dissolution contrast is likely to be obtained. In addition, since the anion moiety of the component (D0) has an iodine atom or a bromine atom (R^{d0}), the absorption with respect to light exposure is increased.

However, the amount of light reaching the bottom portion of the resist film (the interface with the substrate) is likely to be reduced. That is, the amount of the acid to be generated in the resist film is relatively small at the bottom portion of the resist film and relatively large at the upper portion of the resist film. Meanwhile, in the present embodiment, the anion moiety of the component (D0) contains a hydrocarbon group (Rf) which may have a fluorine atom via a linking group (-L¹-L²-L³-) including a divalent cyclic group (L²) which may have a substituent. Therefore, the component (D0) is likely to be unevenly distributed in the upper portion of the resist film. In this manner, a difference in the amount of each acid generated upon light exposure is reduced at the bottom portion and the upper portion of the resist film. In addition, since the component (D0) contains a divalent cyclic group (L²) which may have a substituent, the component (D0) has a relatively bulky structure, the diffusion length of the acid is appropriately suppressed, the affinity for the base material component is also increased, and thus the component (D0) is likely to be uniformly distributed in the resist film.

Further, the component (D0) is designed such that a hydroxy group can be optionally bonded to the benzene ring of the anion moiety. In this manner, the solubility in a developing solution can be controlled.

According to the resist composition of the present embodiment, it is assumed that both high sensitivity and roughness reduction can be achieved and a pattern having a satisfactory shape with high rectangularity can be formed, due to the synergistic action described above.

### (Resist Pattern Formation Method)

A resist pattern formation method according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method by performing processes as described below is an exemplary example.

First, a support is coated with the resist composition of the present embodiment using a spinner or the like, and a bake (post apply bake (PAB)) treatment is performed, for example, under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing or the like with an electron beam without using a mask pattern, a bake treatment (post-exposure bake (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. A bake treatment (post bake) may be conducted after the developing treatment in some cases.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, for example, copper, aluminum, nickel, or gold can be used.

The wavelength to be used for light exposure is not particularly limited and the exposure can be conducted using radiation such as an ArF excimer laser, a KrF excimer laser, an F₂ excimer laser, extreme ultraviolet (EUV) rays, vacuum ultraviolet rays (VUV), electron beams (EB), X-rays, and soft X-rays.

The resist pattern formation method of the present embodiment is particularly useful in a case where the step of exposing the resist film to light includes an operation of exposing the resist film to extreme ultraviolet (EUV) rays or electron beams (EB).

The method of exposing the resist film to light may be general exposure (dry exposure) conducted in air or an inert gas such as nitrogen, or liquid immersion lithography.

The liquid immersion lithography is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, for example, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or in combination of two or more kinds thereof. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the resist pattern formation method of the present embodiment described above, since the above-described resist composition is used, both high sensitivity and roughness reduction can be achieved and a resist pattern having a satisfactory shape with high rectangularity can be formed in a case of forming a resist pattern.

Various materials that are used in the resist composition according to the above-described embodiment and the resist pattern formation method according to the above-described embodiment (for example, a resist solvent, a developing solution, a rinse solution, a composition for forming an antireflection film, and a composition for forming a top coat) preferably do not contain impurities such as a metal, a metal salt containing halogen, an acid, an alkali, and a component containing a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, and particularly preferably 10 ppt or less, where it is most preferable that the impurities are substantially free (less than or equal to the detection limit of the measuring device).

(Compound Represented by General Formula (d0))

A compound according to a third aspect of the present invention is a compound represented by General Formula (d0) (compound (D0)).

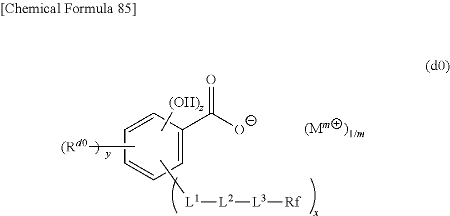

[In the formula, Rf represents a hydrocarbon group which may have a fluorine atom. L² represents a divalent cyclic group which may have a substituent. L¹ and L³ each independently represent a divalent linking group. x represents an integer of 1 to 4. In a case where x represents an integer of 2 or greater, a plurality of (-L¹-L²-L³-Rf)'s may be the same as or different from each other. R^{d0} represents an iodine atom or a bromine atom. y represents an integer of 1 to 4. In a case where y represents an integer of 2 or greater, a plurality of R^{d0}'s may be the same as or different from each other. z represents an integer of 0 to 3. Here, 2≤x+y+z≤5 is satisfied. (M^{m+})_{1/m} represents a counter cation. m represents an integer of 1 or greater.]

The compound represented by General Formula (d0) (compound (D0)) is the same as the component (D0) in the resist composition according to the first aspect of the present invention described above.

The compound of the present embodiment is preferably a compound in which Rf in Formula (d0) represents a fluorinated hydrocarbon group.

Alternatively, the compound of the present embodiment is preferably a compound in which the total number of fluorine atoms contained in x pieces of Rf's in Formula (d0) is 5 or more and 9 or less.

Alternatively, the compound of the present embodiment is preferably a compound in which y in Formula (d0) represents 2 or 3.

Specific examples of the compound of the present embodiment include a compound in which the anion moiety is an anion represented by any of Chemical Formulae (d0-an-101) to (d0-an-113) (in a case where y in Formula (d0) represents 1), a compound in which the anion moiety is an anion represented by any of Chemical Formulae (d0-an-201) to (d0-an-242) (in a case where y in Formula (d0) represents 2), and a compound in which the anion moiety is an anion represented by any of Chemical Formulae (d0-an-301) to (d0-an-310) (in a case where y in Formula (d0) represents 3).

Examples of the counter cation (M^{m+})_{1/m} include an onium cation and a metal cation. Examples of the onium cation include a sulfonium cation, an iodonium cation, and an ammonium cation. Examples of the metal cation include alkali metal ions such as sodium ions and lithium ions, alkaline earth metal ions, rubidium ions, strontium ions, and yttrium ions.

Specific suitable examples of the compound according to the present embodiment include compounds each represented by Chemical Formulae (D0-1) to (D0-24).

[Method of Producing Compound Represented by General Formula (d0)]

The component (D0) can be produced by a known production method.

As an embodiment of the method of producing the component (D0), a production method for a compound in a case where L¹ in General Formula (d0) represents —O—C(═O)— (ester bond) will be described below.

### First Step:

First, a compound (CA0) represented by General Formula (CA0) is used as a starting material, and the compound (CA0) reacts with a tertiary alcohol to obtain a first intermediate (H01) represented by General Formula (H01).

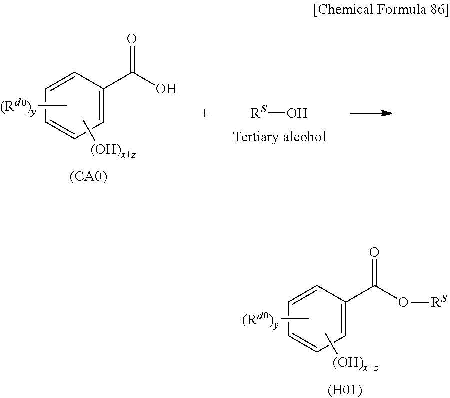

[In the formulae, R^{d0} represents an iodine atom or a bromine atom. y represents an integer of 1 to 4. In a case where y represents an integer of 2 or greater, a plurality of R^{d}0's may be the same as or different from each other. x represents an integer of 1 to 4. z represents an integer of 0 to 3. Here, 2≤x+y+z≤5 is satisfied. R^{S} represents a tertiary alkyl group.]

In the first step, the number of bonds and the bonding positions of the hydroxy group, and the number of bonds and the bonding positions of the iodine atom or the bromine atom (R^{d0}) of the compound (CA0) as a starting material are appropriately selected according to the target compound (component (D0)).

In the formulae, R^{d0}, x, y, and z each have the same definition as that for R^{d0}, x, y, and z in General Formula (d0).

As the tertiary alcohol (R^{S}—OH), tert-butyl alcohol, 1-methylcyclopentanol, and the like can be used.

In the reaction between the compound (CA0) and the tertiary alcohol, a condensing agent is appropriately selected and used. Examples of the condensing agent include dicyclohexylcarbodiimide, diisopropylcarbodiimide (DIC), 1-ethyl-3-(3-dimethylaminopropyl) carbodiimide hydrochloride, and carbonyldiimidazole (CDI).

The organic solvent in a case where the compound (CA0) reacts with a tertiary alcohol may be an organic solvent which can dissolve the compound (CA0), does not react with the compound (CA0), and has satisfactory compatibility with a tertiary alcohol, and examples thereof include dichloromethane, dichloroethane, chloroform, tetrahydrofuran (THF), N,N-dimethylformamide, acetonitrile, propionitrile, N,N′-dimethylacetamide, and dimethyl sulfoxide.

In the first step, the reaction temperature is, for example, in a range of 40° C. to 80° C., and the reaction time is, for example, 1 hour or longer and 10 hours or shorter.

### Second Step:

A second intermediate (H02) represented by General Formula (H02) is prepared.

In Formula (H02), Rf, L², and L³ each have the same definition as that for Rf, L², and L³ in General Formula (d0).

As the second intermediate (H02), a commercially available product may be used in a case of being available, or a synthesized product may be used.

In a case where a synthesized product is used, the second intermediate (H02) can be obtained by alkylating or fluoroalkylating anhydrous acetic acid having the same structure as the structure of the divalent cyclic group (L²) which may have a substituent. Examples of the reagent for alkylation or fluoroalkylation include compounds each represented by Chemical Formulae (e1) to (e10).

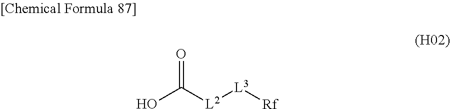

[In the formula, Rf represents a hydrocarbon group which may have a fluorine atom. L² represents a divalent cyclic group which may have a substituent. L³ represents a divalent linking group.]

### Third Step:

Next, the first intermediate (H01) and the second intermediate (H02) react with each other in the presence of a base to obtain a third intermediate (H03) represented by General Formula (H03).

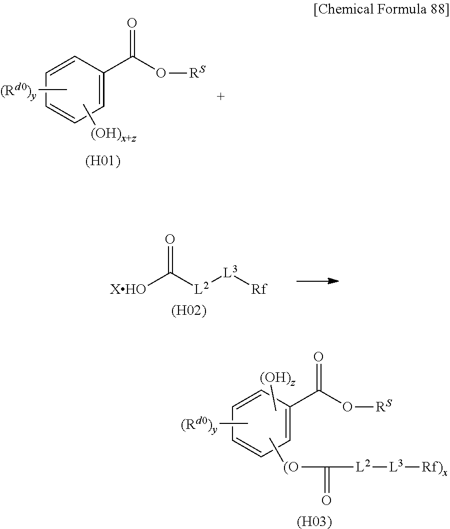

[In the formulae, R^{d0} represents an iodine atom or a bromine atom. y represents an integer of 1 to 4. In a case where y represents an integer of 2 or greater, a plurality of R^{d0}'s may be the same as or different from each other. x represents an integer of 1 to 4. z represents an integer of 0 to 3. Here, 2≤x+y+z≤5 is satisfied. R^{S} represents a tertiary alkyl group.

Rf represents a hydrocarbon group which may have a fluorine atom. L² represents a divalent cyclic group which may have a substituent. L³ represents a divalent linking group.]

In the third step, the structure and the like of the second intermediate (H02) are appropriately selected according to the target compound (component (D0)).

In the formulae, R^{d0}, x, y, z, Rf, L², and L³ each have the same definition as that for R^{d0}, x, y, z, Rf, L², and L³ in General Formula (d0).

Specific examples of the base include sodium hydride, K₂CO₃, Cs₂CO₃, lithium diisopropylamide (LDA), triethylamine, and 4-dimethylaminopyridine.

In the reaction between the first intermediate (H01) and the second intermediate (H02), a condensing agent is appropriately selected and used. Examples of the condensing agent include dicyclohexylcarbodiimide, diisopropylcarbodiimide (DIC), 1-ethyl-3-(3-dimethylaminopropyl) carbodiimide hydrochloride, and carbonyldiimidazole (CDI).

The organic solvent in a case of reacting the first intermediate (H01) with the second intermediate (H02) may be any organic solvent that can dissolve the first intermediate (H01) and the second intermediate (H02) and does not react with the first intermediate (H01) and the second intermediate (H02), and examples thereof include dichloromethane, dichloroethane, chloroform, tetrahydrofuran, N,N-dimethylformamide, acetonitrile, propionitrile, N,N′-dimethylacetamide, and dimethyl sulfoxide.

In the third step, the reaction temperature is, for example, in a range of 0° C. to 50° C., and the reaction time is, for example, 1 hour or longer and 24 hours or shorter.

### Fourth Step:

Next, the third intermediate (H03) is hydrolyzed to obtain a precursor (D′0).

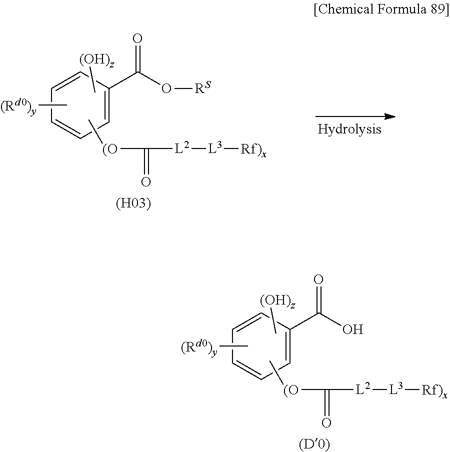

[In the formulae, R^{d0} represents an iodine atom or a bromine atom. y represents an integer of 1 to 4. In a case where y represents an integer of 2 or greater, a plurality of R^{d0}'s may be the same as or different from each other. x represents an integer of 1 to 4. z represents an integer of 0 to 3. Here, 2≤x+y+z≤5 is satisfied. R^{S} represents a tertiary alkyl group.

Rf represents a hydrocarbon group which may have a fluorine atom. L² represents a divalent cyclic group which may have a substituent. L³ represents a divalent linking group.]

In the hydrolysis of the third intermediate (H03), an acid can be used as a catalyst. As this acid, for example, trifluoroacetic acid (TFA), methanesulfonic acid, or the like can be used.

The organic solvent in a case of hydrolyzing the third intermediate (H03) may be any organic solvent that can dissolve the third intermediate and the acid catalyst and does not react with the third intermediate and the acid catalyst, and examples thereof include dichloromethane, dichloroethane, chloroform, tetrahydrofuran, N,N-dimethylformamide, acetonitrile, propionitrile, N,N′-dimethylacetamide, and dimethyl sulfoxide.

In the fourth step, the reaction temperature is, for example, in a range of 10° C. to 60° C., and the reaction time is, for example, 1 hour or longer and 24 hours or shorter.

### Fifth Step:

Next, a target compound represented by General Formula (d0′) is obtained by carrying out a salt exchange reaction between the precursor (D′0) and a compound (S0) represented by General Formula (S0).

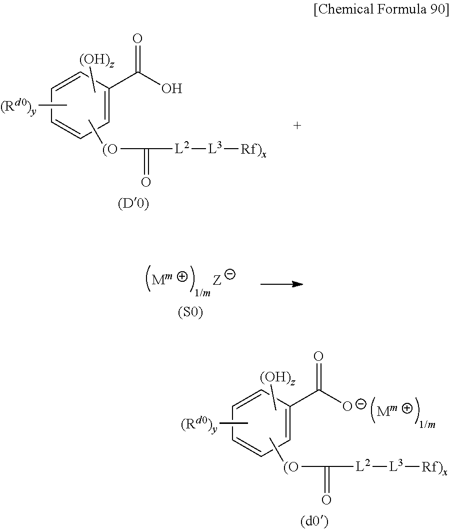

[In the formulae, R^{d0} represents an iodine atom or a bromine atom. y represents an integer of 1 to 4. In a case where y represents an integer of 2 or greater, a plurality of R^{d0}'s may be the same as or different from each other. x represents an integer of 1 to 4. z represents an integer of 0 to 3. Here, 2≤x+y+z≤5 is satisfied. R^{S} represents a tertiary alkyl group.

Rf represents a hydrocarbon group which may have a fluorine atom. L² represents a divalent cyclic group which may have a substituent. L³ represents a divalent linking group.

M^{m+} represents an m-valent cation. m represents an integer of 1 or greater. Z-represents a counter anion.]

In the formula, examples of the counter anion as Z⁻ include ions that can be an acid having a lower acidity than that of a precursor represented by General Formula (D′0), and specific examples thereof include a halogen ion such as a bromine ion or a chloride ion; and BF₄⁻, AsF₆⁻, SbF₆⁻, PF₆⁻, and ClO₄⁻. Among these, Z⁻ represents preferably a halogen ion and more preferably a bromine ion.

In the formulae, M^{m+} and m each have the same definition as that for M^{m+} and m in General Formula (d0).

In the fifth step, a base may be used in the salt exchange reaction between the precursor (D′0) and the compound (S0). Examples of the base include tetramethylammonium hydroxide (TMAH), sodium hydride, K₂CO₃, Cs₂CO₃, lithium diisopropylamide (LDA), triethylamine, and 4-dimethylaminopyridine.

The solvent in a case of carrying out the salt exchange reaction between the precursor (D′0) and the compound (S0) may be an organic solvent or a mixed solvent of an organic solvent and water. Examples of the organic solvent include a ketone-based solvent such as cyclohexanone, methyl ethyl ketone, diethyl ketone, or methyl isobutyl ketone, an ether-based solvent such as diethyl ether, t-butyl methyl ether, or diisopropyl ether, a halogen-based solvent such as tetrahydrofuran, 1,3-dioxolane, dichloromethane, or 1,2-dichloroethane, an ester-based solvent such as ethyl acetate or propylene glycol monomethyl ether acetate, propionitrile, and a mixed solvent thereof.

In the fifth step, the reaction temperature is, for example, in a range of 0° C. to 50° C., and the reaction time is, for example, 10 minutes or longer and 5 hours or shorter.

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by general organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

As raw materials, reagents, and the like used in each step, commercially available products may be used, or synthesized products may be used.

In the above-described method of producing the component (D0), the method of producing a compound in a case where L¹ in General Formula (d0) represents —O—C(═O)-(ester bond) has been described. However, for example, in the method of producing a compound in a case where L¹ in General Formula (d0) represents —C(═O)—NH— (amide bond), a compound in which x pieces of hydroxy groups (—OH) in the compound (CA0) are replaced with an amino group (—NH₂) may be used as a starting material.

The compound according to the present embodiment described above is a suitable compound as the base component for a resist composition according to the first aspect of the present invention described above. In addition, the compound according to the present embodiment can be used as an acid diffusion control agent according to a fourth aspect described below.

### (Acid Diffusion Control Agent)

An acid diffusion control agent according to the fourth aspect of the present invention contains the above-described compound (compound (D0)) according to the above-described third aspect.

The acid diffusion control agent of the present embodiment is particularly suitable as the base component for the resist composition according to the first aspect.

Such an acid diffusion control agent is useful as an acid diffusion control agent for a chemically amplified resist composition.

Since the compound according to the third aspect described above has a carboxylate anion in the anion moiety, an acid with relatively weak acid strength as compared with a fluorinated alkylsulfonate anion or the like contained in an anion moiety of an acid generator that is typically used in a chemically amplified resist composition is generated upon light exposure.

In a case where such an acid diffusion control agent is used in the chemically amplified resist composition, the lithography characteristics such as the pattern shape (rectangularity) and roughness reduction are further improved in the resist pattern formation. Particularly in the resist pattern formation using EB or an EUV light source, the lithography characteristics such as the sensitivity, the pattern shape (rectangularity), and the roughness reduction are further improved by using such an acid diffusion control agent.

### EXAMPLES

Hereinafter, the present invention will be described in more detail based on examples, but the present invention is not limited to these examples.

In present Examples, a compound represented by Chemical Formula (X-1-1) is denoted by a “compound (X-1-1)”, and compounds represented by other chemical formulae are also denoted in the same manner.

### <Production of First Intermediate>

### Production Example 1-1

A three-neck flask having a volume of 300 mL was charged with 1,1′-carbonyldiimidazole (CDI) (8.60 g, 41.7 mmol) and tetrahydrofuran (THF) (20 g), a solution obtained by dissolving 4-iodosalicylic acid (ca1) (10.0 g, 37.9 mmol) in THF (20 g) was added dropwise thereto over 30 minutes, and the solution was stirred for 1 hour. Thereafter, tert-butyl alcohol (3.37 g, 45.5 mmol) was added to the solution, and the solution was allowed to react at 65° C. for 3 hours. After the solution was cooled, ultrapure water (250 g) and methyl tert-butyl ether (MTBE) (150 g) were added thereto, the solution was stirred for 30 minutes, and the aqueous layer was removed. Thereafter, the organic layer was washed with ultrapure water (100 g) three times, the solvent was distilled off from the organic layer, the obtained crude product was dissolved again in THF (20 g), methanol (80 g) was added dropwise thereto, and the precipitated solid was filtered and dried under reduced pressure, thereby obtaining 9.46 g of a first intermediate (pre-a01).

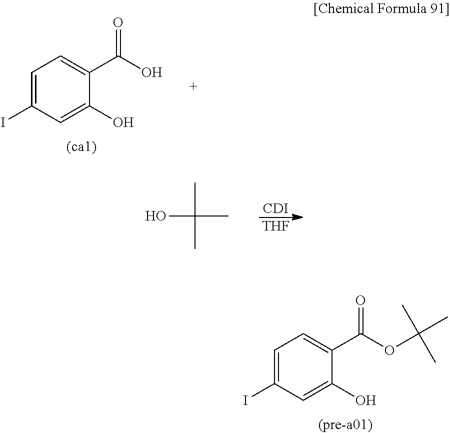

### Production Examples 1-2 to 1-7

4-lodosalicylic acid (ca1) (10.0 g, 37.9 mmol) serving as a starting material was changed to each of the following starting materials (ca2) to (ca7) at the equimolar ratio to obtain first intermediates (pre-a02) to (pre-a07) in the same manner as in (Production Example 1-1) described above.

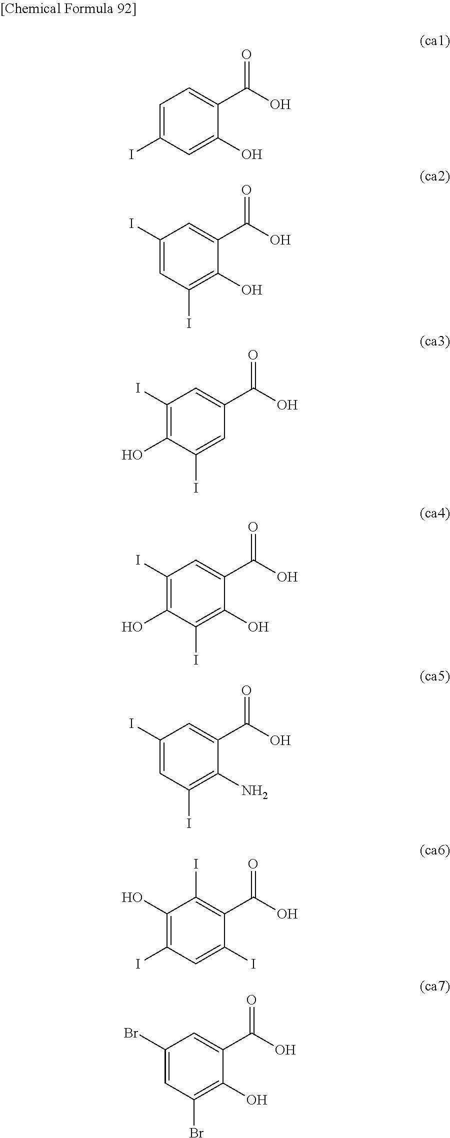

The obtained first intermediates (pre-a01) to (pre-a07) are shown below.

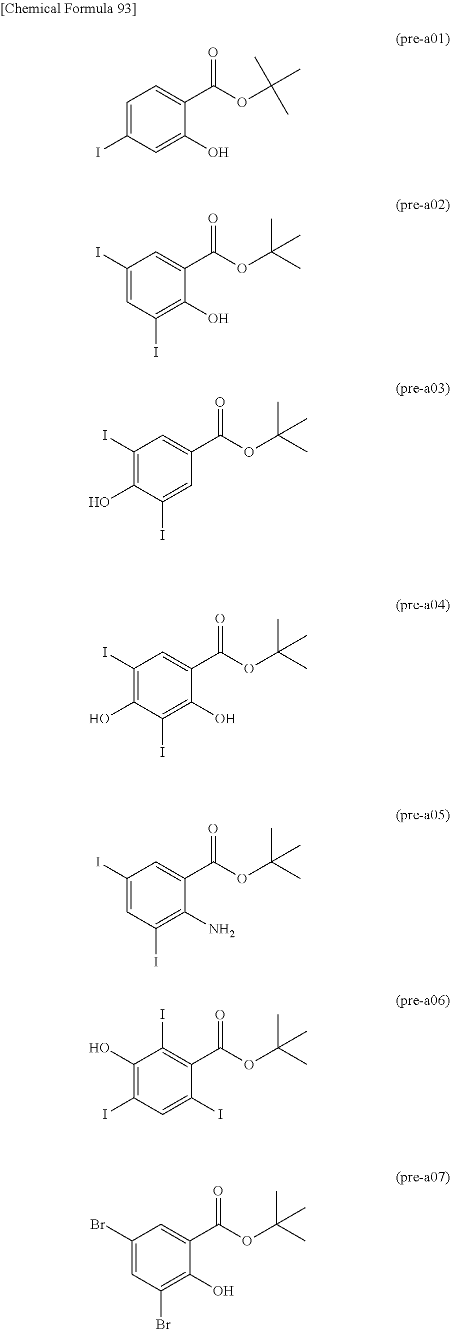

### <Production of Second Intermediate>

### Production Example 2-1

A three-neck flask having a volume of 300 mL was charged with 60% sodium hydride (NaH) (2.92 g, 73.1 mmol) and THF (30 g), 1-butanol (e1) (4.51 g, 60.9 mmol) was added dropwise thereto over 30 minutes under ice cooling, and the mixture was allowed to react for 30 minutes. Thereafter, a solution obtained by dissolving a 5-norbornene-2,3-dicarboxylic anhydride (da1) (10.0 g, 60.9 mmol) in THF (20 g) was added dropwise thereto over 20 minutes, and the solution was stirred at room temperature for 7 hours. The reaction solution was added dropwise to a 5 wt % hydrochloric acid aqueous solution (64.33 g) under ice cooling, the reaction solution was stirred for 1 hour, and the aqueous layer was removed. Thereafter, the solvent was distilled off from the organic layer, the obtained crude product was recrystallized in methanol, and the precipitated solid was filtered and dried under reduced pressure, thereby obtaining 8.7 g of a second intermediate (pre-b01).

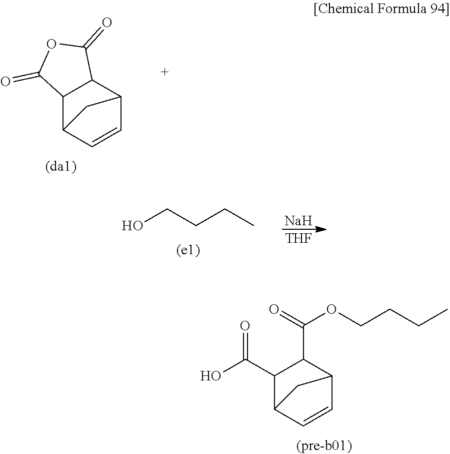

### Production Examples 2-2 to 2-11

Second intermediates (pre-b02) to (pre-b11) were obtained in the same manner as in (Production Example 2-1) described above by reacting the following dicarboxylic anhydride (da1), the following dicarboxylic anhydride (da2), and the following reagents (e2) to (e10) in the combinations shown below.

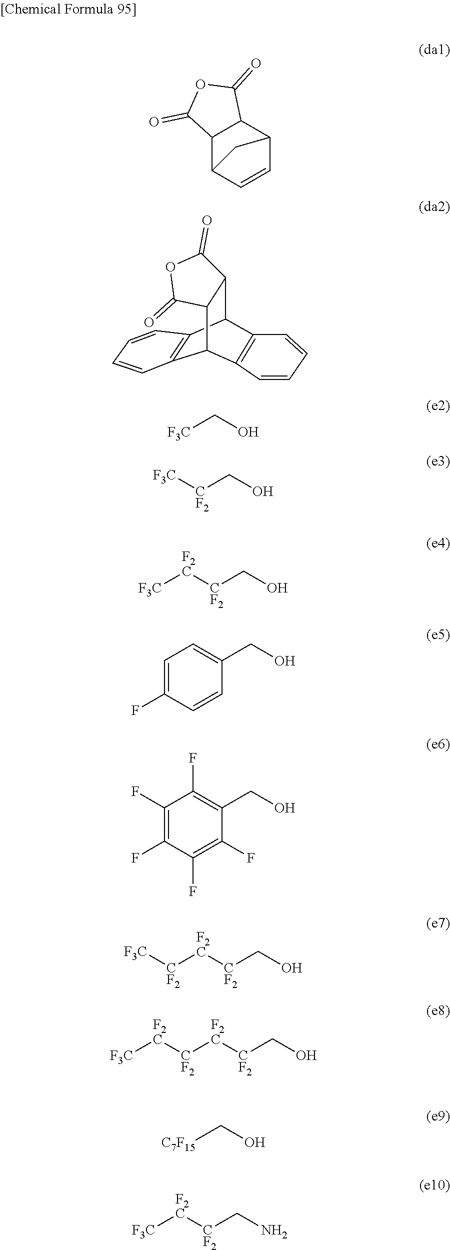

Second intermediate (pre-b02): combination of dicarboxylic anhydride (da1) and reagent (e2)
Second intermediate (pre-b03): combination of dicarboxylic anhydride (da1) and reagent (e3)
Second intermediate (pre-b04): combination of dicarboxylic anhydride (da1) and reagent (e4)
Second intermediate (pre-b05): combination of dicarboxylic anhydride (da1) and reagent (e5)
Second intermediate (pre-b06): combination of dicarboxylic anhydride (da1) and reagent (e6)
Second intermediate (pre-b07): combination of dicarboxylic anhydride (da1) and reagent (e7)
Second intermediate (pre-b08): combination of dicarboxylic anhydride (da1) and reagent (e8)
Second intermediate (pre-b09): combination of dicarboxylic anhydride (da1) and reagent (e9)
Second intermediate (pre-b10): combination of dicarboxylic anhydride (da1) and reagent (e10)
Second intermediate (pre-b11): combination of dicarboxylic anhydride (da2) and reagent (e4)

The obtained second intermediates (pre-b01) to (pre-b11) are shown below.

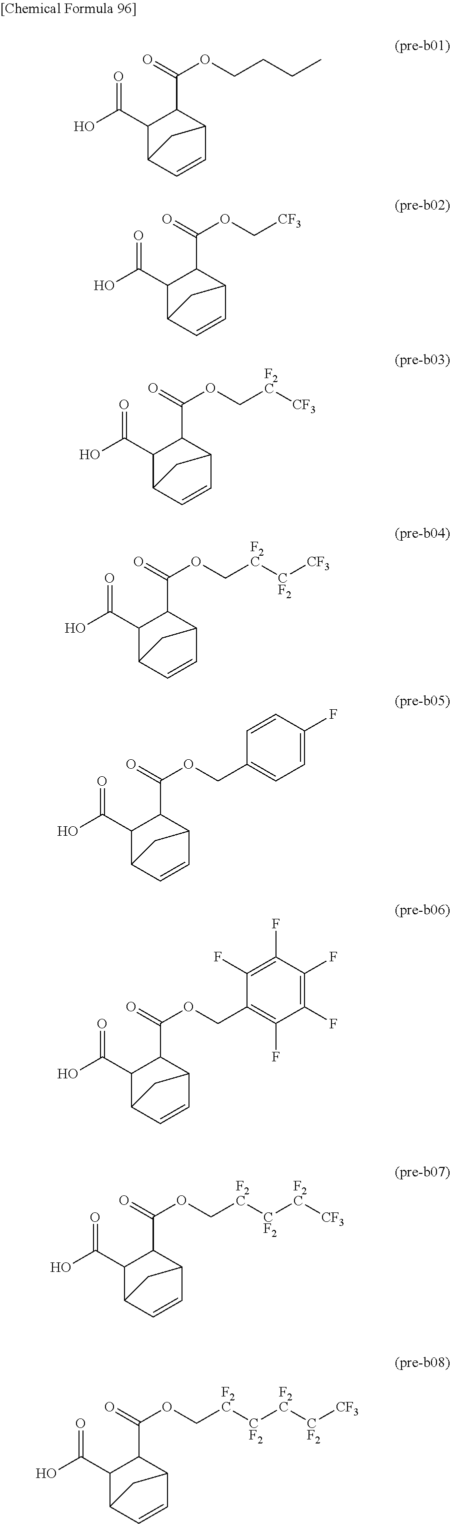

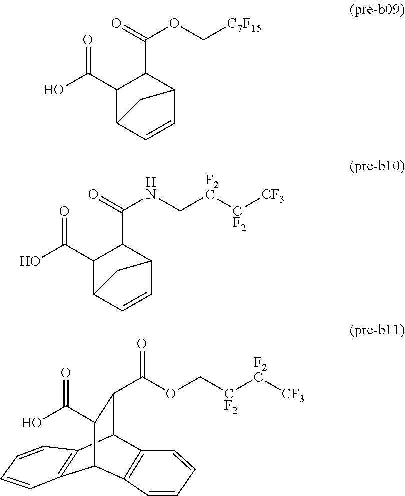

### Production Example 2-12

A three-neck flask having a volume of 200 mL was charged with 2-((2,2,3,3,4,4,4-heptafluorobutyl)oxy)acetic acid (20.0 g, 77.5 mmol), thionyl chloride (18.4 g, 155 mmol), and N,N-dimethylformamide (DMF) (40 g), and the mixture was stirred at 70° C. for 4 hours. Thereafter, 15.4 g of a compound (cac1) was obtained by distilling off the solvent.

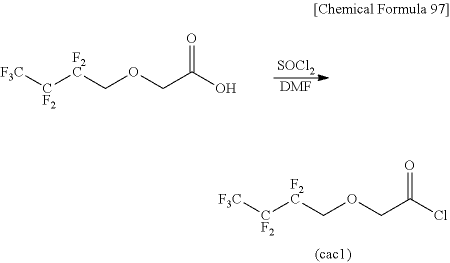

A three-neck flask having a volume of 500 mL was charged with 3-hydroxy-1-adamantanecarboxylic acid (7.8 g, 40.0 mmol), THU (54.6 g), and triethylamine (6.1 g), a solution obtained by dissolving the compound (cac1) (12.1 g, 44.1 mmol) in THF (15.6 g) was added dropwise thereto under ice cooling, and the solution was stirred at room temperature for 3 hours. The solvent of the reaction solution was distilled off, the concentrate was dissolved in dichloromethane (80 g), and the solution was washed with a 5 wt % hydrochloric acid aqueous solution (80 g) twice and with ultrapure water (80 g) three times, and the solvent was distilled off from the organic layer. The obtained crude product was recrystallized with methanol, and the precipitated solid was filtered and dried under reduced pressure, thereby obtaining 14.1 g of a second intermediate (pre-b12).

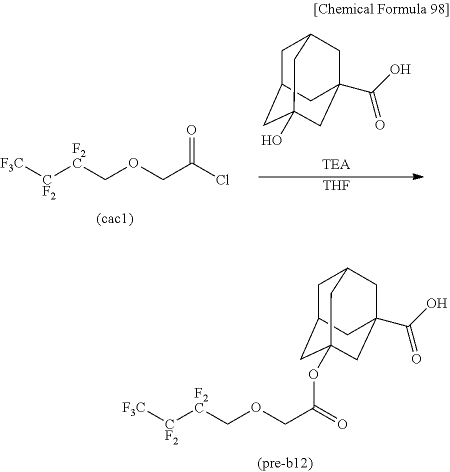 [SMILES: O=C(Cl)COCC(F)(F)C(F)(F)C(F)(F)F ; O=C(O)C12CC3CC(CC(O)(C3)C1)C2 ; O=C(COCC(F)(F)C(F)(F)C(F)(F)F)OC12CC3CC(C1)CC(C(=O)O)(C3)C2 ; "THF"]

### <Production of Third Intermediate>

### Production Example 3-1

A three-neck flask having a volume of 300 mL was charged with the second intermediate (pre-b01) (7.1 g, 30.0 mmol), dichloromethane (71 g), diisopropylcarbodiimide (4.5 g, 36.0 mmol), and dimethylaminopyridine (0.37 g, 3.0 mmol), and the mixture was stirred at room temperature for 30 minutes. Thereafter, the first intermediate (pre-a01) (9.12 g, 28.5 mmol) was added thereinto, and the mixture was stirred at room temperature for 5 hours. The reaction solution was filtered, the solvent was distilled off from the filtrate, the concentrate was dissolved in acetonitrile (16 g) and methanol (32 g), MTBE (160 g) was added dropwise thereto, and the precipitated solid was filtered. After this operation was repeated three times, the residue was dried under reduced pressure, thereby obtaining 12.0 g of a third intermediate (pre-c01).

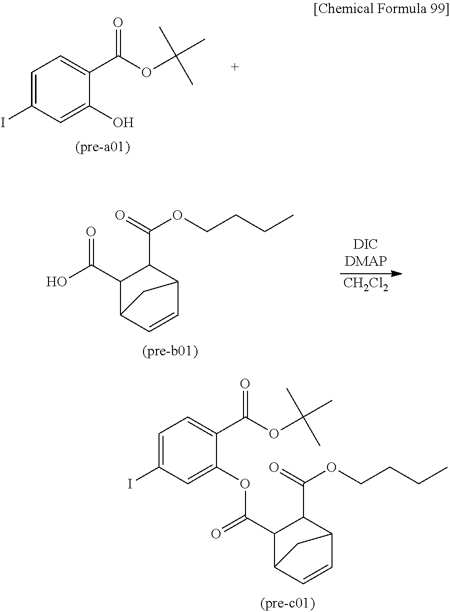 [SMILES: CC(C)(C)OC(=O)c1ccc(I)cc1O ; CCCCOC(=O)C1C2C=CC(C2)C1C(=O)O ; "CH2Cl2" ; CCCCOC(=O)C1C2C=CC(C2)C1C(=O)Oc1cc(I)ccc1C(=O)OC(C)(C)C]

### Production Examples 3-2 to 3-21

Third intermediates (pre-c02) to (pre-c21) were obtained by reacting the first intermediates (pre-a01) to (pre-a07) and second intermediates (pre-b01) to (pre-b12) in the combinations shown below in the same manner as in (Production Example 3-1) described above.
Third intermediate (pre-c02): combination of first intermediate (pre-a01) and second intermediate (pre-b02)
Third intermediate (pre-c03): combination of first intermediate (pre-a01) and second intermediate (pre-b04)
Third intermediate (pre-c04): combination of first intermediate (pre-a02) and second intermediate (pre-b01)
Third intermediate (pre-c05): combination of first intermediate (pre-a02) and second intermediate (pre-b05)
Third intermediate (pre-c06): combination of first intermediate (pre-a02) and second intermediate (pre-b02)
Third intermediate (pre-c07): combination of first intermediate (pre-a02) and second intermediate (pre-b03)
Third intermediate (pre-c08): combination of first intermediate (pre-a02) and second intermediate (pre-b06)
Third intermediate (pre-c09): combination of first intermediate (pre-a02) and second intermediate (pre-b04)
Third intermediate (pre-c10): combination of first intermediate (pre-a02) and second intermediate (pre-b07)
Third intermediate (pre-c11): combination of first intermediate (pre-a02) and second intermediate (pre-b08)
Third intermediate (pre-c12): combination of first intermediate (pre-a02) and second intermediate (pre-b09)
Third intermediate (pre-c13): combination of first intermediate (pre-a03) and second intermediate (pre-b04)
Third intermediate (pre-c14): combination of first intermediate (pre-a04) and second intermediate (pre-b04)
Third intermediate (pre-c15): combination of first intermediate (pre-a05) and second intermediate (pre-b04)
Third intermediate (pre-c16): combination of first intermediate (pre-a05) and second intermediate (pre-b10)
Third intermediate (pre-c17): combination of first intermediate (pre-a02) and second intermediate (pre-b111)
Third intermediate (pre-c18): combination of first intermediate (pre-a02) and second intermediate (pre-b12)
Third intermediate (pre-c19): combination of first intermediate (pre-a06) and second intermediate (pre-b02)
Third intermediate (pre-c20): combination of first intermediate (pre-a06) and second intermediate (pre-b04)
Third intermediate (pre-c21): combination of first intermediate (pre-a07) and second intermediate (pre-b07)

The obtained third intermediates (pre-c01) to (pre-c21) are shown below.

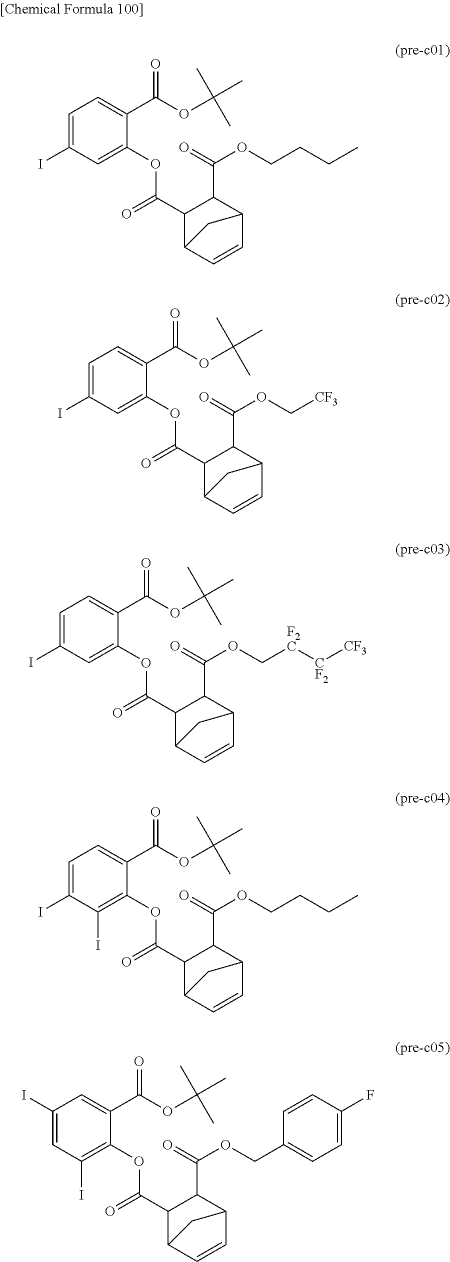 [SMILES: CCCCOC(=O)C1C2C=CC(C2)C1C(=O)Oc1cc(I)ccc1C(=O)OC(C)(C)C ; CC(C)(C)OC(=O)c1ccc(I)cc1OC(=O)C1C2C=CC(C2)C1C(=O)OCC(F)(F)F ; CC(C)(C)OC(=O)c1ccc(I)cc1OC(=O)C1C2C=CC(C2)C1C(=O)OCC(F)(F)C(F)(F)C(F)(F)F ; CCCCOC(=O)C1C2C=CC(C2)C1C(=O)Oc1c(C(=O)OC(C)(C)C)ccc(I)c1I ; CC(C)(C)OC(=O)c1cc(I)cc(I)c1OC(=O)C1C2C=CC(C2)C1C(=O)OCc1ccc(F)cc1]

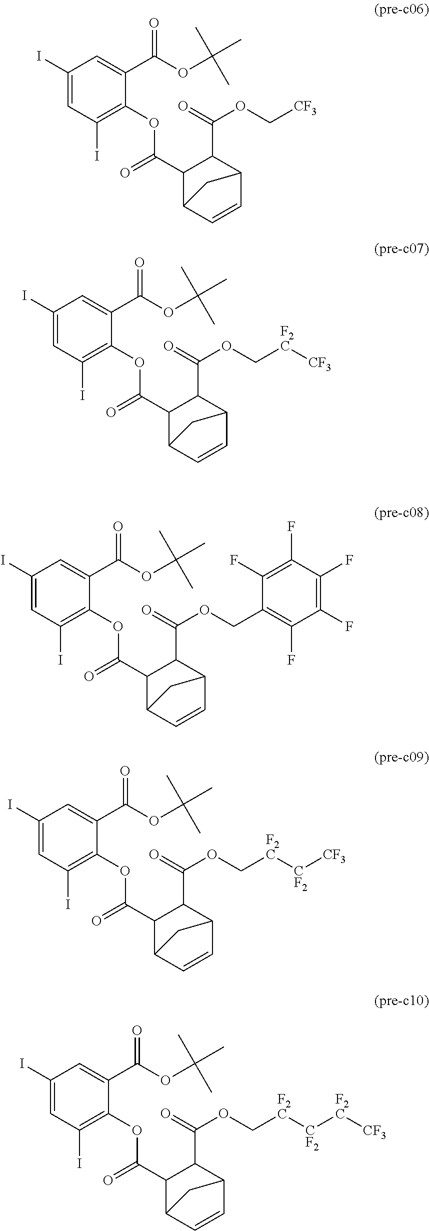 [SMILES: CC(C)(C)OC(=O)c1cc(I)cc(I)c1OC(=O)C1C2C=CC(C2)C1C(=O)OCC(F)(F)F ; CC(C)(C)OC(=O)c1cc(I)cc(I)c1OC(=O)C1C2C=CC(C2)C1C(=O)OCC(F)(F)C(F)(F)F ; CC(C)(C)OC(=O)c1cc(I)cc(I)c1OC(=O)C1C2C=CC(C2)C1C(=O)OCc1c(F)c(F)c(F)c(F)c1F ; CC(C)(C)OC(=O)c1cc(I)cc(I)c1OC(=O)C1C2C=CC(C2)C1C(=O)OCC(F)(F)C(F)(F)C(F)(F)F ; CC(C)(C)OC(=O)c1cc(I)cc(I)c1OC(=O)C1C2C=CC(C2)C1C(=O)OCC(F)(F)C(F)(F)C(F)(F)C(F)(F)F]

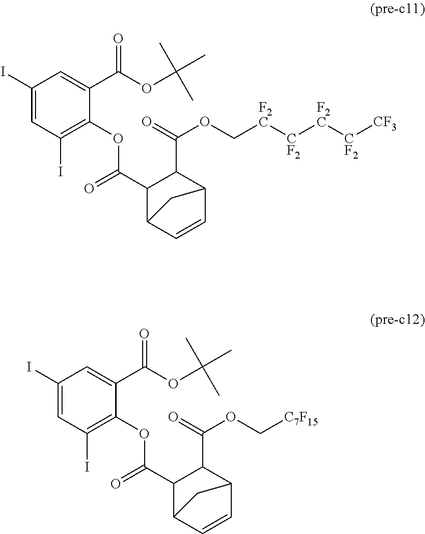

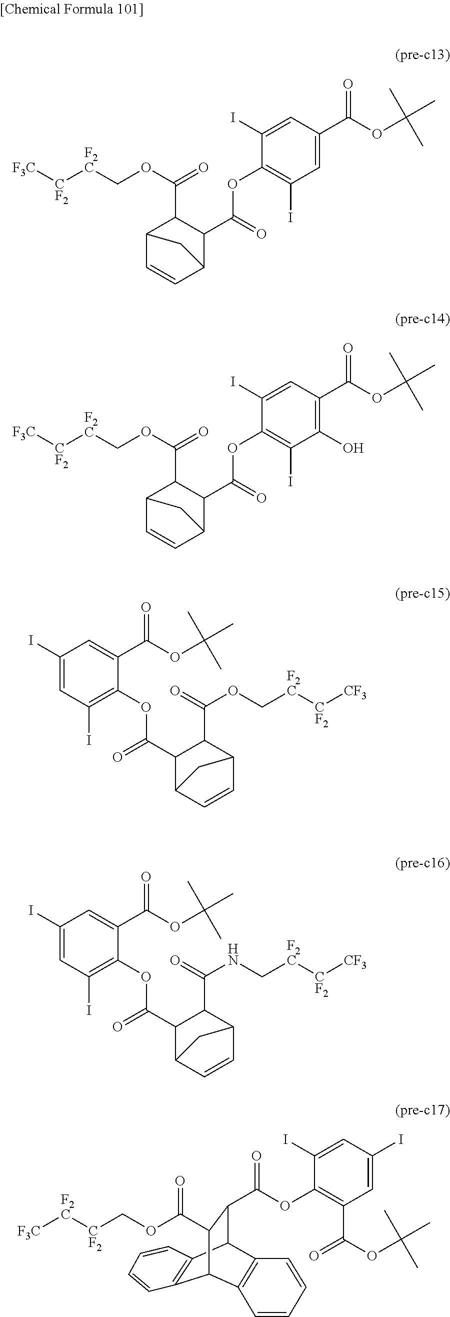 [SMILES: CC(C)(C)OC(=O)c1cc(I)c(OC(=O)C2C3C=CC(C3)C2C(=O)OCC(F)(F)C(F)(F)C(F)(F)F)c(I)c1 ; CC(C)(C)OC(=O)c1cc(I)c(OC(=O)C2C3C=CC(C3)C2C(=O)OCC(F)(F)C(F)(F)C(F)(F)F)c(I)c1O ; CC(C)(C)OC(=O)c1cc(I)cc(I)c1OC(=O)C1C2C=CC(C2)C1C(=O)OCC(F)(F)C(F)(F)C(F)(F)F ; CC(C)(C)OC(=O)c1cc(I)cc(I)c1OC(=O)C1C2C=CC(C2)C1C(=O)NCC(F)(F)C(F)(F)C(F)(F)F ; CC(C)(C)OC(=O)c1cc(I)cc(I)c1OC(=O)C1C2c3ccccc3C(c3ccccc32)C1C(=O)OCC(F)(F)C(F)(F)C(F)(F)F]

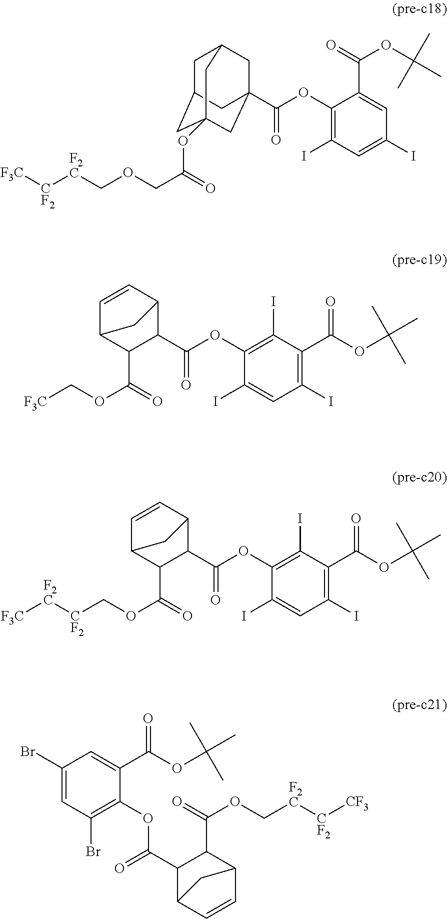 [SMILES: CC(C)(C)OC(=O)c1cc(I)cc(I)c1OC(=O)C12CC3CC(CC(OC(=O)COCC(F)(F)C(F)(F)C(F)(F)F)(C3)C1)C2 ; CC(C)(C)OC(=O)c1c(I)cc(I)c(OC(=O)C2C3C=CC(C3)C2C(=O)OCC(F)(F)F)c1I ; CC(C)(C)OC(=O)c1c(I)cc(I)c(OC(=O)C2C3C=CC(C3)C2C(=O)OCC(F)(F)C(F)(F)C(F)(F)F)c1I ; CC(C)(C)OC(=O)c1cc(Br)cc(Br)c1OC(=O)C1C2C=CC(C2)C1C(=O)OCC(F)(F)C(F)(F)C(F)(F)F]

### <Production of Precursor (D′)>

### Production Example 4-1

A three-neck flask having a volume of 200 mL was charged with the third intermediate (pre-c01) (10.8 g, 20.0 mmol), dichloromethane (100 g), and trifluoroacetic acid (TFA) (22.8 g, 200 mmol), and the mixture was stirred at 30° C. for 8 hours. After the reaction, ultrapure water (100 g) was added thereto, the solution was stirred for 30 minutes, and the aqueous layer was removed. Thereafter, the organic layer was washed with ultrapure water (100 g) three times, and the solvent was distilled off from the organic layer, thereby obtaining 8.8 g of a precursor (D′-01).

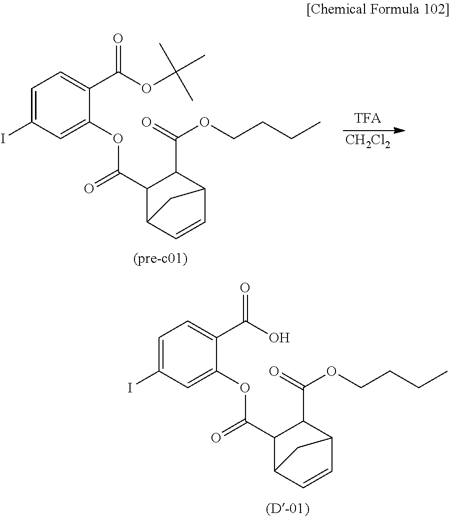

### Production Examples 4-2 to 4-21

Precursors (D′-02) to (D′-21) were obtained by reacting each of the third intermediates (pre-c02) to (pre-c21) with trifluoroacetic acid (TFA) in the same manner as in (Production Example 4-1).

The obtained precursors (D′-01) to (D′-21) are shown below.

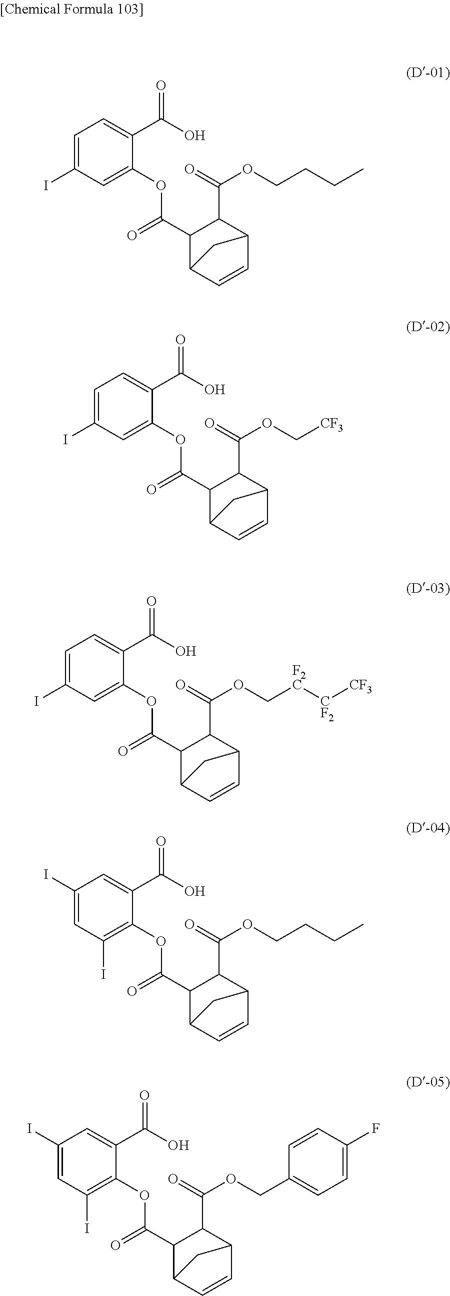 [SMILES: CCCCOC(=O)C1C2C=CC(C2)C1C(=O)Oc1cc(I)ccc1C(=O)O ; O=C(O)c1ccc(I)cc1OC(=O)C1C2C=CC(C2)C1C(=O)OCC(F)(F)F ; O=C(O)c1ccc(I)cc1OC(=O)C1C2C=CC(C2)C1C(=O)OCC(F)(F)C(F)(F)C(F)(F)F ; CCCCOC(=O)C1C2C=CC(C2)C1C(=O)Oc1c(I)cc(I)cc1C(=O)O ; O=C(O)c1cc(I)cc(I)c1OC(=O)C1C2C=CC(C2)C1C(=O)OCc1ccc(F)cc1]

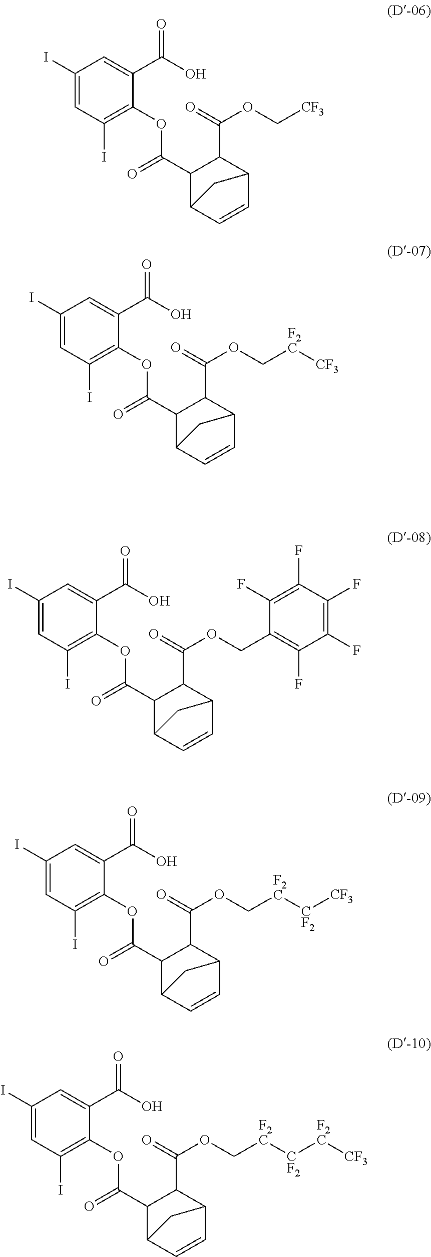 [SMILES: O=C(O)c1cc(I)cc(I)c1OC(=O)C1C2C=CC(C2)C1C(=O)OCC(F)(F)F ; O=C(O)c1cc(I)cc(I)c1OC(=O)C1C2C=CC(C2)C1C(=O)OCC(F)(F)C(F)(F)F ; O=C(O)c1cc(I)cc(I)c1OC(=O)C1C2C=CC(C2)C1C(=O)OCc1c(F)c(F)c(F)c(F)c1F ; O=C(O)c1cc(I)cc(I)c1OC(=O)C1C2C=CC(C2)C1C(=O)OCC(F)(F)C(F)(F)C(F)(F)F ; O=C(O)c1cc(I)cc(I)c1OC(=O)C1C2C=CC(C2)C1C(=O)OCC(F)(F)C(F)(F)C(F)(F)C(F)(F)F]

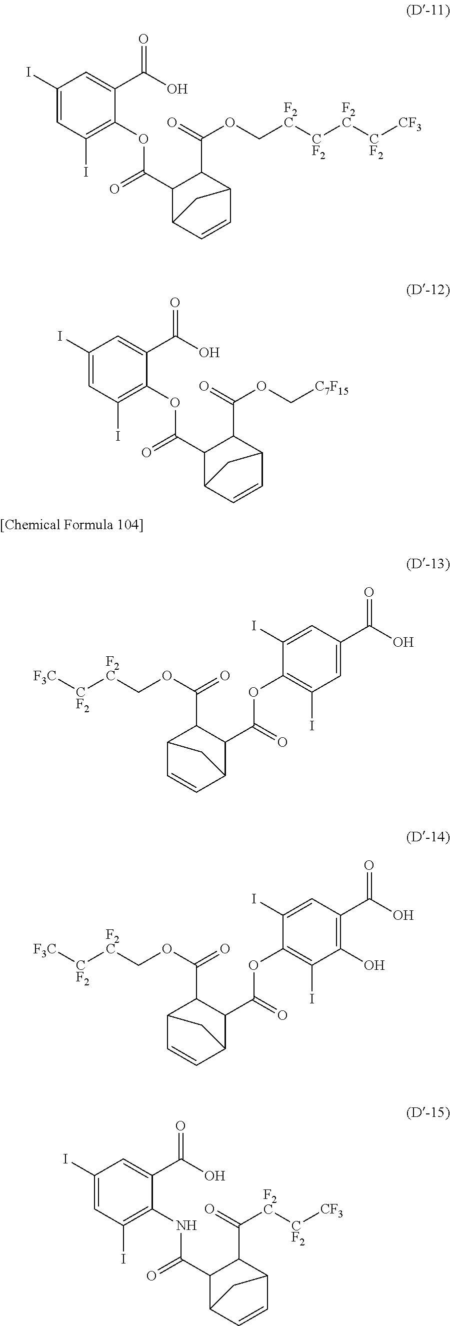

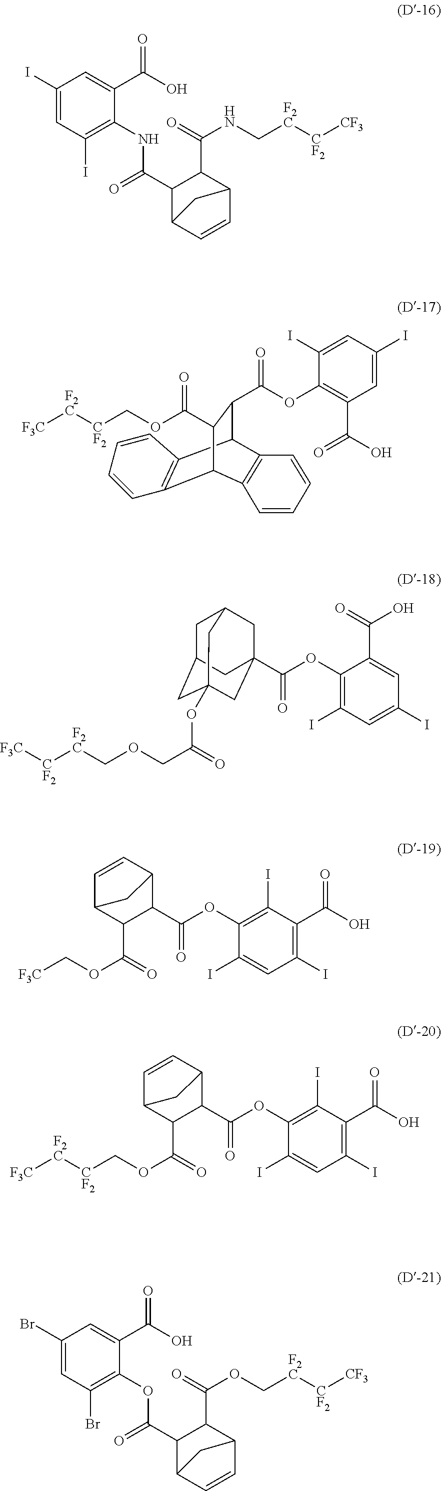 [SMILES: O=C(O)c1cc(I)cc(I)c1NC(=O)C1C2C=CC(C2)C1C(=O)NCC(F)(F)C(F)(F)C(F)(F)F ; O=C(O)c1cc(I)cc(I)c1OC(=O)C1C2c3ccccc3C(c3ccccc32)C1C(=O)OCC(F)(F)C(F)(F)C(F)(F)F ; O=C(COCC(F)(F)C(F)(F)C(F)(F)F)OC12CC3CC(C1)CC(C(=O)Oc1c(I)cc(I)cc1C(=O)O)(C3)C2 ; O=C(O)c1c(I)cc(I)c(OC(=O)C2C3C=CC(C3)C2C(=O)OCC(F)(F)F)c1I ; O=C(O)c1c(I)cc(I)c(OC(=O)C2C3C=CC(C3)C2C(=O)OCC(F)(F)C(F)(F)C(F)(F)F)c1I ; O=C(O)c1cc(Br)cc(Br)c1OC(=O)C1C2C=CC(C2)C1C(=O)OCC(F)(F)C(F)(F)C(F)(F)F]

<Production of compound (D0)>

### Production Example 5-1

The precursor (D′-01) (6.0 g, 12.4 mmol) and the compound A (4.7 g, 13.6 mmol) were dissolved in dichloromethane (50 g), a 5% tetramethylannonium hydroxide (TMAH) aqueous solution (25 g) was added thereto, and the solution was allowed to react at room temperature for 30 minutes. After completion of the reaction, the aqueous phase was removed, and the organic phase was washed with ultrapure water (50 g) five times. 8.4 g of a compound (D0-1) was obtained by distilling off the solvent from the organic phase.

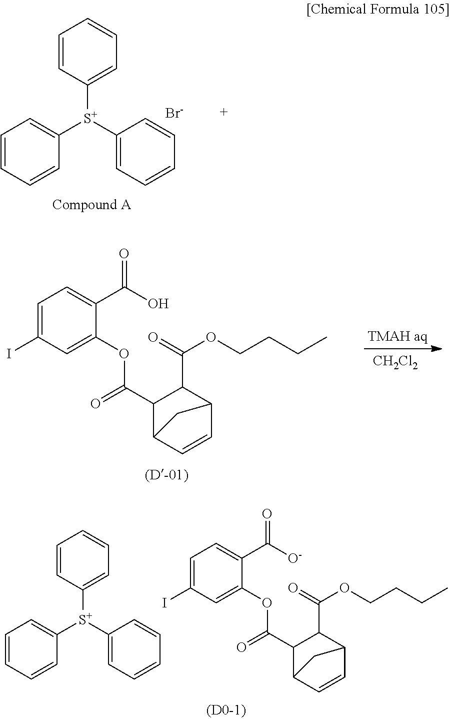

### Production Examples 5-2 to 5-24

Target compounds (D0-2) to (D0-24) were obtained by reacting the precursors (D′-02) to (D′-21) and the following compounds A to D for salt exchange in the combinations shown below in the same manner as in (Production Example 5-1).

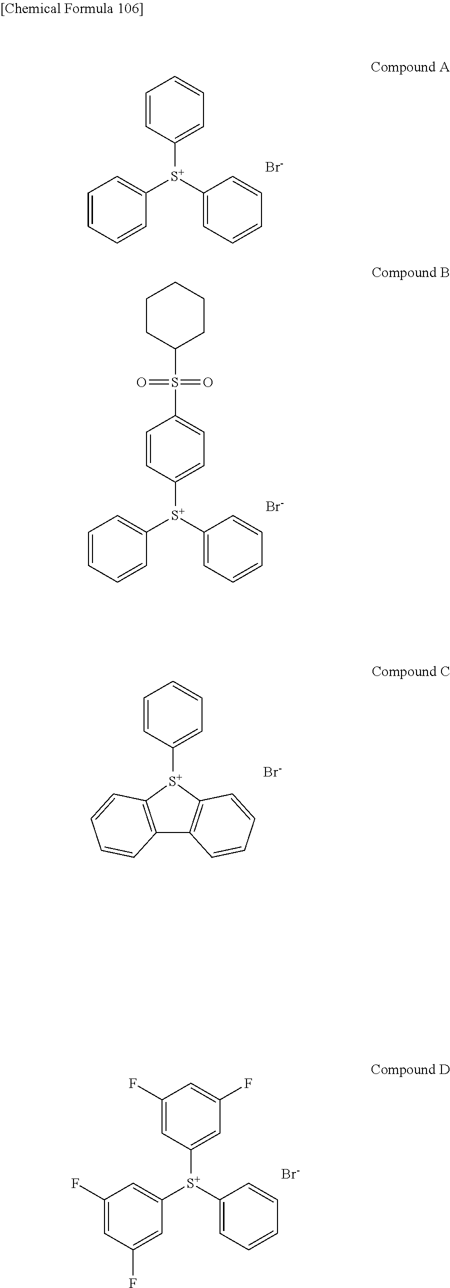

Each of the obtained compounds was subjected to NMR measurement, and the structure thereof was identified from the following analysis results.

Compound (D0-1): combination of precursor (D′-01) and compound A for salt exchange

¹H-NMR (DMSO, 400 MHz): δ (ppm)=7.91 (1H, dd), 7.72 (1H, d), 7.62 (1H, d), 7.42-7.28 (15H, m), 6.08-6.02 (2H, m), 4.06 (2H, t), 3.75-3.63 (2H, m), 3.04-2.96 (2H, m), 1.79-1.71 (1H, m), 1.58-1.30 (5H, m), 0.90 (3H, t)

Compound (D0-2): combination of precursor (D′-02) and compound A for salt exchange

¹H-NMR (DMSO, 400 MHz): δ (ppm)=7.91 (1H, dd), 7.72 (1H, d), 7.62 (1H, d), 7.42-7.28 (15H, m), 6.08-6.02 (2H, m), 4.60 (2H, dd), 3.75-3.63 (2H, m), 3.03-2.97 (2H, m), 1.79-1.71 (1H, m), 1.54-1.46 (1H, m)

Compound (D0-3): combination of precursor (D′-03) and compound A for salt exchange

¹H-NMR (DMSO, 400 MHz): δ (ppm)=7.91 (1H, dd), 7.72 (1H, d), 7.62 (1H, d), 7.42-7.28 (15H, m), 6.09-6.02 (2H, m), 4.39 (2H, t), 3.76-3.64 (2H, m), 3.03-2.97 (2H, m), 1.80-1.72 (1H, m), 1.55-1.47 (1H, m)

Compound (D0-4): combination of precursor (D′-04) and compound A for salt exchange

¹H-NMR (DMSO, 400 MHz): δ (ppm)=8.30 (1H, d), 8.18 (1H, d), 7.42-7.28 (15H, m), 6.08-6.02 (2H, m), 4.06 (2H, t), 3.75-3.63 (2H, m), 3.04-2.96 (2H, m), 1.79-1.71 (1H, m), 1.58-1.30 (5H, m), 0.90 (3H, t)

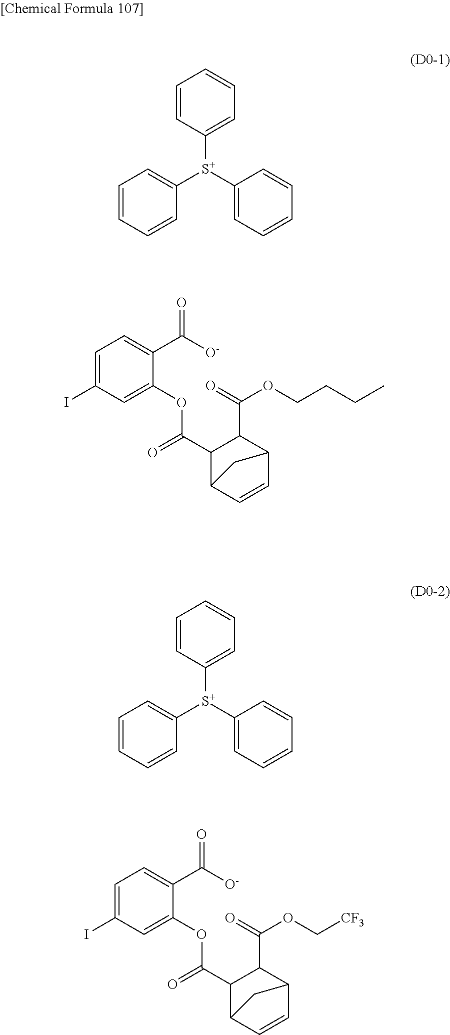 [SMILES: c1ccc([S+](c2ccccc2)c2ccccc2)cc1 ; CCCCOC(=O)C1C2C=CC(C2)C1C(=O)Oc1cc(I)ccc1C(=O)[O-] ; c1ccc([S+](c2ccccc2)c2ccccc2)cc1 ; O=C([O-])c1ccc(I)cc1OC(=O)C1C2C=CC(C2)C1C(=O)OCC(F)(F)F]

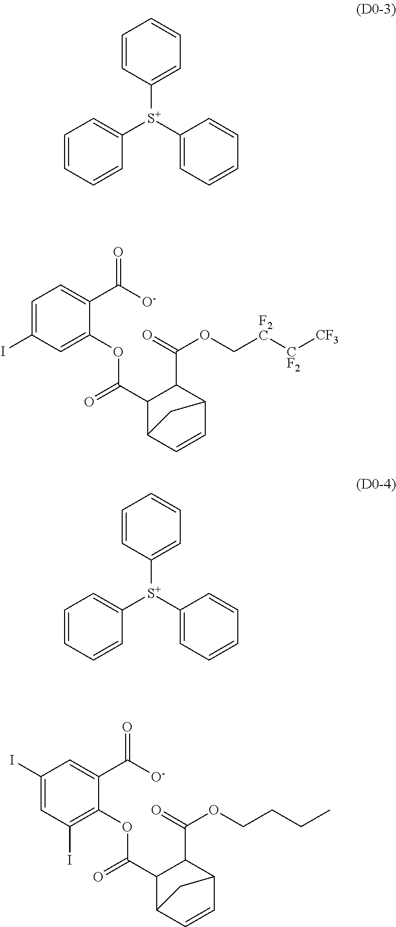 [SMILES: c1ccc([S+](c2ccccc2)c2ccccc2)cc1 ; O=C([O-])c1ccc(I)cc1OC(=O)C1C2C=CC(C2)C1C(=O)OCC(F)(F)C(F)(F)C(F)(F)F ; c1ccc([S+](c2ccccc2)c2ccccc2)cc1 ; CCCCOC(=O)C1C2C=CC(C2)C1C(=O)Oc1c(I)cc(I)cc1C(=O)[O-]]

Compound (D0-5): combination of precursor (D′-05) and compound A for salt exchange

¹H-NMR (DMSO, 400 MHz): δ (ppm)=8.30 (1H, d), 8.18 (1H, d), 7.43-7.29 (15H, m), 7.26-7.19 (4H, m), 6.09-6.01 (2H, m), 5.34 (2H, s), 3.75-3.62 (2H, m), 3.04-2.98 (2H, m), 1.79-1.71 (1H, m), 1.54-1.46 (1H, m)

Compound (D0-6): combination of precursor (D′-06) and compound A for salt exchange

¹H-NMR (DMSO, 400 MHz): δ (ppm)=8.30 (1H, d), 8.18 (1H, d), 7.42-7.28 (15H, m), 6.08-6.02 (2H, m), 4.38 (2H, t), 3.75-3.63 (2H, m), 3.03-2.97 (2H, m), 1.80-1.72 (1H, m), 1.55-1.47 (1H, m)

Compound (D0-7): combination of precursor (D′-07) and compound A for salt exchange

¹H-NMR (DMSO, 400 MHz): δ (ppm)=8.30 (1H, d), 8.18 (1H, d), 7.42-7.28 (15H, m), 6.09-6.01 (2H, m), 4.42 (2H, t), 3.75-3.63 (2H, m), 3.03-2.97 (2H, m), 1.80-1.72 (1H, m), 1.55-1.47 (1H, m)

Compound (D0-8): combination of precursor (D′-08) and compound A for salt exchange

¹H-NMR (DMSO, 400 MHz): δ (ppm)=8.30 (1H, d), 8.18 (1H, d), 7.42-7.28 (15H, m), 6.09-6.01 (2H, m), 5.64 (2H, s), 3.75-3.63 (2H, m), 3.03-2.97 (2H, m), 1.79-1.71 (1H, m), 1.54-1.46 (1H, m)

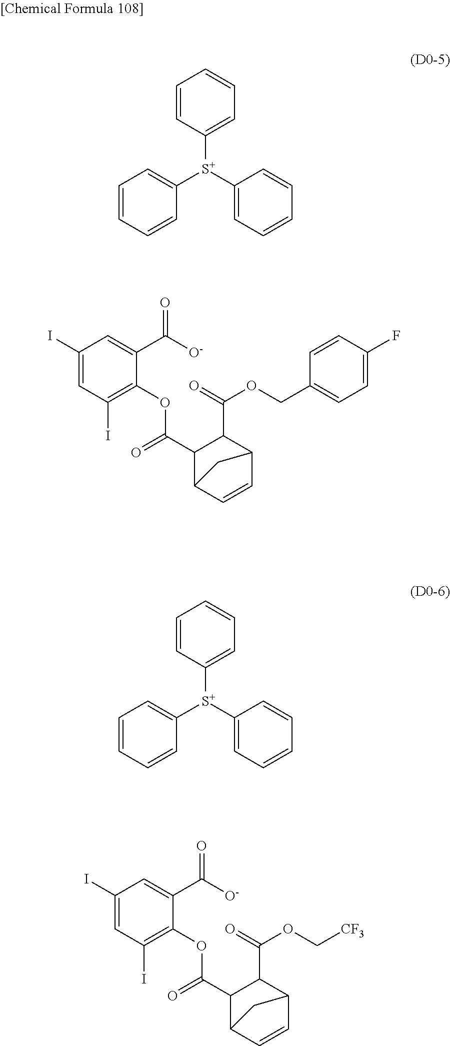 [SMILES: c1ccc([S+](c2ccccc2)c2ccccc2)cc1 ; O=C([O-])c1cc(I)cc(I)c1OC(=O)C1C2C=CC(C2)C1C(=O)OCc1ccc(F)cc1 ; c1ccc([S+](c2ccccc2)c2ccccc2)cc1 ; O=C([O-])c1cc(I)cc(I)c1OC(=O)C1C2C=CC(C2)C1C(=O)OCC(F)(F)F]

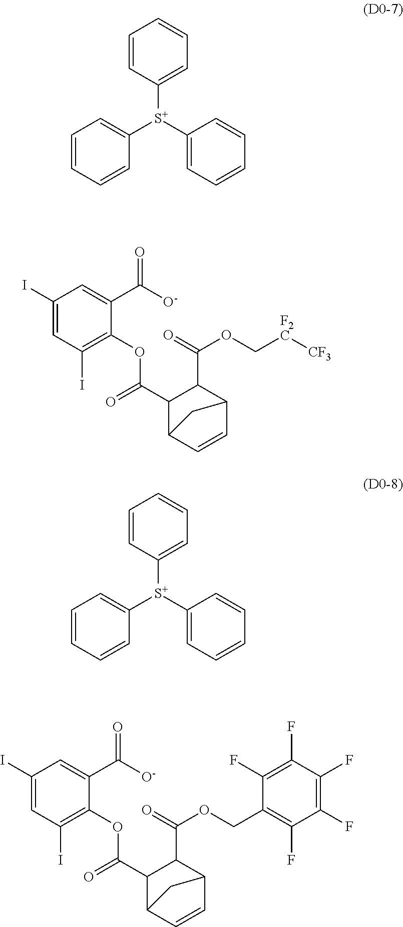 [SMILES: c1ccc([S+](c2ccccc2)c2ccccc2)cc1 ; O=C([O-])c1cc(I)cc(I)c1OC(=O)C1C2C=CC(C2)C1C(=O)OCC(F)(F)C(F)(F)F ; c1ccc([S+](c2ccccc2)c2ccccc2)cc1 ; O=C([O-])c1cc(I)cc(I)c1OC(=O)C1C2C=CC(C2)C1C(=O)OCc1c(F)c(F)c(F)c(F)c1F]

Compound (D0-9): combination of precursor (D′-09) and compound A for salt exchange

¹H-NMR (DMSO, 400 MHz): δ (ppm)=8.30 (1H, d), 8.18 (1H, d), 7.42-7.28 (15H, m), 6.09-6.02 (2H, m), 4.43 (2H, t), 3.75-3.63 (2H, m), 3.03-2.97 (2H, m), 1.80-1.71 (1H, m), 1.55-1.47 (1H, m)

Compound (D0-10): combination of precursor (D′-10) and compound A for salt exchange

¹H-NMR (DMSO, 400 MHz): δ (ppm)=8.30 (1H, d), 8.18 (1H, d), 7.42-7.28 (15H, m), 6.10-6.01 (2H, m), 4.42 (2H, t), 3.75-3.63 (2H, m), 3.03-2.97 (2H, m), 1.79-1.71 (1H, m), 1.54-1.46 (1H, m)

Compound (D0-11): combination of precursor (D′-11) and compound A for salt exchange

¹H-NMR (DMSO, 400 MHz): δ (ppm)=8.31 (1H, d), 8.18 (1H, d), 7.42-7.28 (15H, m), 6.08-6.02 (2H, m), 4.38 (2H, t), 3.75-6.63 (2H, m), 3.03-2.97 (2H, m), 1.79-1.71 (1H, m), 1.54-1.46 (1H, m)

Compound (D0-12): combination of precursor (D′-12) and compound A for salt exchange

¹H-NMR (DMSO, 400 MHz): δ (ppm)=8.30 (1H, d), 8.19 (1H, d), 7.42-7.28 (15H, m), 6.08-6.02 (2H, m), 4.38 (2H, t), 3.75-3.63 (2H, m), 3.03-2.97 (2H, m), 1.80-1.71 (1H, m), 1.54-1.46 (1H, m)

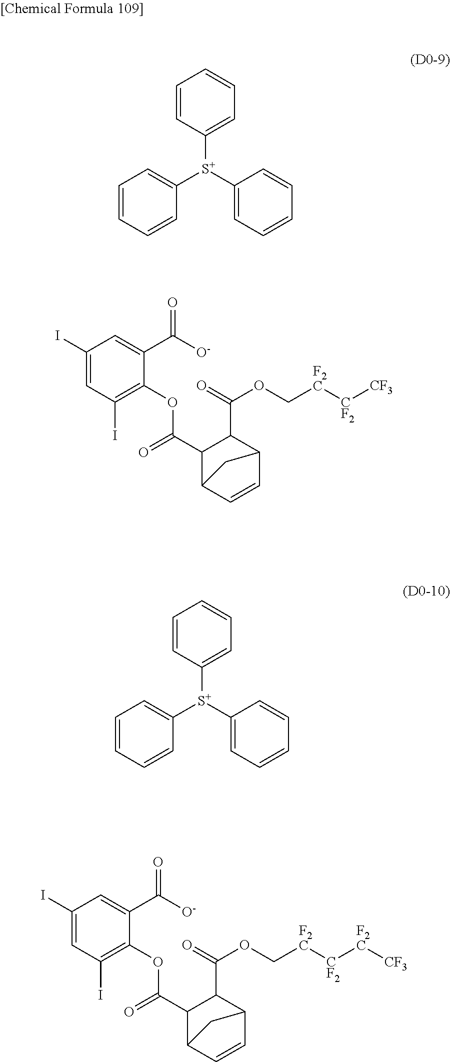 [SMILES: c1ccc([S+](c2ccccc2)c2ccccc2)cc1 ; O=C([O-])c1cc(I)cc(I)c1OC(=O)C1C2C=CC(C2)C1C(=O)OCC(F)(F)C(F)(F)C(F)(F)F ; c1ccc([S+](c2ccccc2)c2ccccc2)cc1 ; O=C([O-])c1cc(I)cc(I)c1OC(=O)C1C2C=CC(C2)C1C(=O)OCC(F)(F)C(F)(F)C(F)(F)C(F)(F)F]

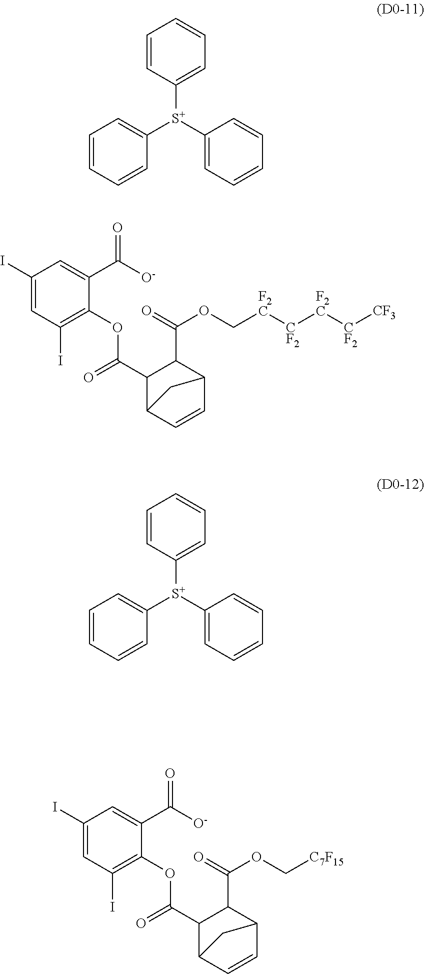

Compound (D0-13): combination of precursor (D′-13) and compound A for salt exchange

¹H-NMR (DMSO, 400 MHz): δ (ppm)=8.18 (2H, s), 7.42-7.28 (15H, m), 6.27-6.19 (2H, m), 4.38 (2H, t), 3.76-3.64 (2H, m), 3.04-2.98 (2H, m), 1.79-1.71 (1H, m), 1.54-1.46 (1H, m)

Compound (D0-14): combination of precursor (D′-14) and compound A for salt exchange

¹H-NMR (DMSO, 400 MHz): δ (ppm)=21.38 (1H, brs), 8.01 (1H, s), 7.42-7.28 (15H, m), 6.26-6.20 (2H, m), 4.39 (2H, t), 3.76-3.64 (2H, m), 3.03-2.97 (2H, m), 1.79-1.71 (1H, m), 1.54-1.46 (1H, m)

Compound (D0-15): combination of precursor (D′-15) and compound A for salt exchange

¹H-NMR (DMSO, 400 MHz): δ (ppm)=10.02 (1H, brs), 8.40 (1H, d), 8.28 (1H, d), 7.42-7.28 (15H, m), 6.26-6.20 (2H, m), 4.38 (2H, t), 3.74-3.61 (2H, m), 3.41-3.31 (1H, m), 2.67 (1H, t), 1.79-1.71 (1H, m), 1.54-1.46 (1H, m)

Compound (D0-16): combination of precursor (D′-16) and compound A for salt exchange

¹H-NMR (DMSO, 400 MHz): δ (ppm)=10.02 (1H, brs), 8.40 (1H, d), 8.28 (1H, d), 8.01 (1H, brs), 7.42-7.28 (15H, m), 6.25-6.21 (2H, m), 3.50 (2H, t), 3.43-3.29 (2H, m), 2.94 (1H, t), 2.78 (1H, t), 1.79-1.71 (1H, m), 1.54-1.46 (1H, m)

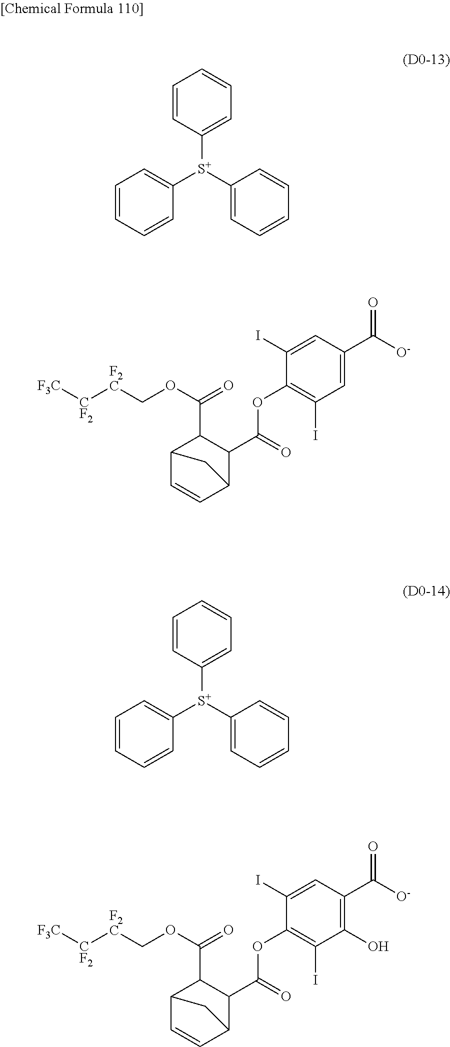 [SMILES: c1ccc([S+](c2ccccc2)c2ccccc2)cc1 ; O=C([O-])c1cc(I)c(OC(=O)C2C3C=CC(C3)C2C(=O)OCC(F)(F)C(F)(F)C(F)(F)F)c(I)c1 ; c1ccc([S+](c2ccccc2)c2ccccc2)cc1 ; O=C([O-])c1cc(I)c(OC(=O)C2C3C=CC(C3)C2C(=O)OCC(F)(F)C(F)(F)C(F)(F)F)c(I)c1O]

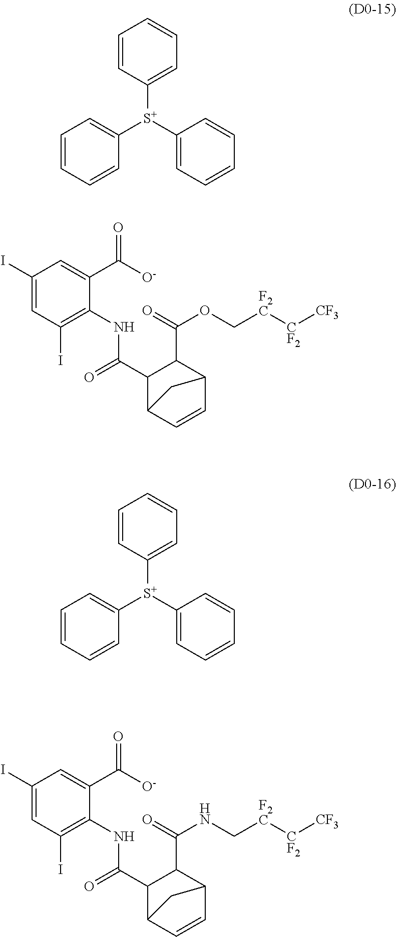 [SMILES: c1ccc([S+](c2ccccc2)c2ccccc2)cc1 ; O=C([O-])c1cc(I)cc(I)c1NC(=O)C1C2C=CC(C2)C1C(=O)OCC(F)(F)C(F)(F)C(F)(F)F ; c1ccc([S+](c2ccccc2)c2ccccc2)cc1 ; O=C([O-])c1cc(I)cc(I)c1NC(=O)C1C2C=CC(C2)C1C(=O)NCC(F)(F)C(F)(F)C(F)(F)F]

Compound (D0-17): combination of precursor (D′-17) and compound A for salt exchange

¹H-NMR (DMSO, 400 MHz): δ (ppm)=8.31 (1H, d), 8.20 (1H, d), 7.42-7.14 (23H, m), 4.48-3.34 (4H, m), 3.48-3.40 (2H, m)

Compound (D0-18): combination of precursor (D′-18) and compound A for salt exchange

¹H-NMR (DMSO, 400 MHz): δ (ppm)=8.29 (1H, d), 8.18 (1H, d), 7.42-7.28 (15H, m), 4.33 (2H, s), 3.67 (2H, t), 2.05 (1H, s), 1.83-1.69 (4H, m), 1.58-1.25 (6H, m), 1.19-1.04 (3H, m)

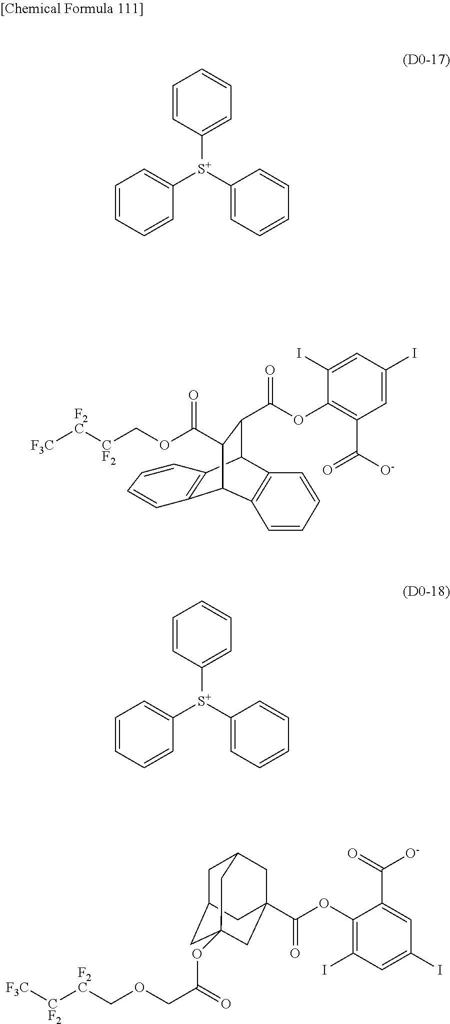 [SMILES: c1ccc([S+](c2ccccc2)c2ccccc2)cc1 ; O=C([O-])c1cc(I)cc(I)c1OC(=O)C1C2c3ccccc3C(c3ccccc32)C1C(=O)OCC(F)(F)C(F)(F)C(F)(F)F ; c1ccc([S+](c2ccccc2)c2ccccc2)cc1 ; O=C(COCC(F)(F)C(F)(F)C(F)(F)F)OC12CC3CC(C1)CC(C(=O)Oc1c(I)cc(I)cc1C(=O)[O-])(C3)C2]

Compound (D0-19): combination of precursor (D′-19) and compound A for salt exchange

¹H-NMR (DMSO, 400 MHz): δ (ppm)=8.20 (1H, s), 7.42-7.28 (15H, m), 6.26-6.20 (2H, m), 4.60 (2H, dd), 3.76-3.62 (2H, m), 3.03-2.97 (2H, m), 1.79-1.71 (1H, m), 1.54-1.46 (1H, m)

Compound (D0-20): combination of precursor (D′-20) and compound A for salt exchange

¹H-NMR (DMSO, 400 MHz): δ (ppm)=8.20 (1H, s), 7.42-7.28 (15H, m), 6.27-6.20 (2H, m), 4.38 (2H, t), 3.76-3.62 (2H, m), 3.03-2.97 (2H, m), 1.79-1.71 (1H, m), 1.54-1.46 (1H, m)

Compound (D0-21): combination of precursor (D′-21) and compound A for salt exchange

¹H-NMR (DMSO, 400 MHz): δ (ppm)=8.23 (1H, d), 7.97 (1H, d), 7.42-7.28 (15H, m), 6.26-6.20 (2H, m), 4.38 (2H, t), 3.74-3.60 (2H, m), 3.05-2.98 (2H, m), 1.79-1.71 (1H, m), 1.54-1.46 (1H, m)

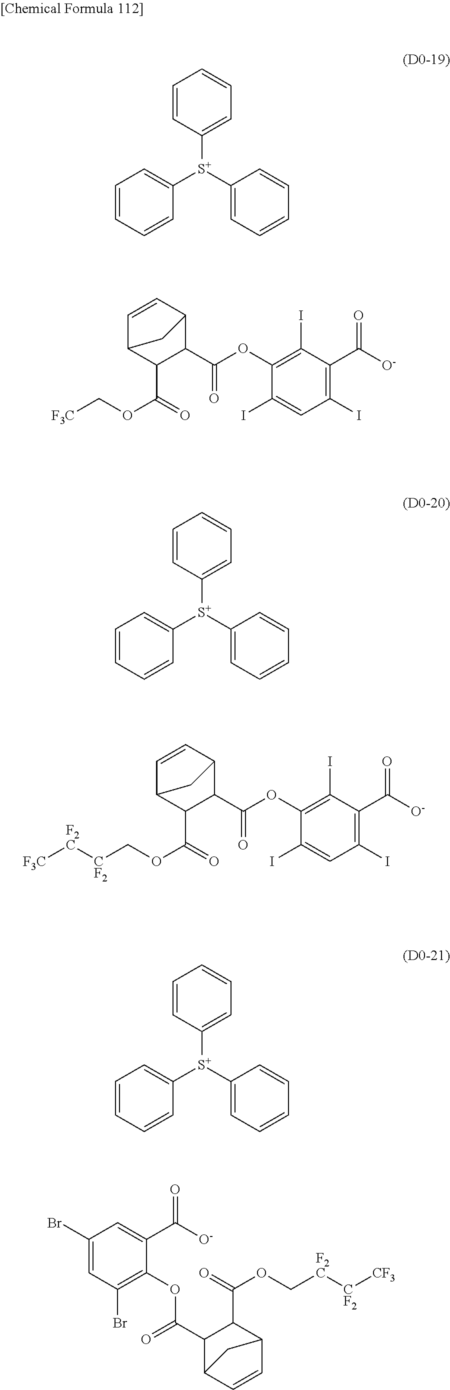 [SMILES: c1ccc([S+](c2ccccc2)c2ccccc2)cc1 ; O=C([O-])c1c(I)cc(I)c(OC(=O)C2C3C=CC(C3)C2C(=O)OCC(F)(F)F)c1I ; c1ccc([S+](c2ccccc2)c2ccccc2)cc1 ; O=C([O-])c1c(I)cc(I)c(OC(=O)C2C3C=CC(C3)C2C(=O)OCC(F)(F)C(F)(F)C(F)(F)F)c1I ; c1ccc([S+](c2ccccc2)c2ccccc2)cc1 ; O=C([O-])c1cc(Br)cc(Br)c1OC(=O)C1C2C=CC(C2)C1C(=O)OCC(F)(F)C(F)(F)C(F)(F)F]

Compound (D0-22): combination of precursor (D′-09) and compound B for salt exchange

¹H-NMR (DMSO, 400 MHz): δ (ppm)=8.30 (1H, d), 8.20 (1H, d), 7.80 (2H d), 7.68 (2H, d), 7.41-7.30 (10H, m), 6.09-6.02 (2H, m), 4.43 (2H, t), 3.75-3.63 (2H, m), 3.03-2.92 (3H, m), 2.15-2.03 (2H, m), 1.91-1.71 (3H, m), 1.60-1.36 (7H, m)

Compound (D0-23): combination of precursor (D′-09) and compound C for salt exchange

¹H-NMR (DMSO, 400 MHz): δ (ppm)=8.30 (1H, d), 8.18 (1H, d), 7.80-7.74 (2H, m), 7.56-7.44 (6H, m), 7.40-7.30 (5H, m), 6.10-6.02 (2H, m), 4.44 (2H, t), 3.75-3.62 (2H, m), 3.03-2.97 (2H, m), 1.80-1.71 (1H, m), 1.55-1.47 (1H, m)

Compound (D0-24): combination of precursor (D′-09) and compound D for salt exchange

¹H-NMR (DMSO, 400 MHz): δ (ppm)=8.30 (1H, d), 8.19 (1H, d), 7.41-7.30 (5H, m), 6.71-6.83 (6H, m), 6.09-6.02 (2H, m), 4.43 (2H, t), 3.74-3.63 (2H, m), 3.03-2.96 (2H, m), 1.80-1.71 (1H, m), 1.55-1.48 (1H, m)

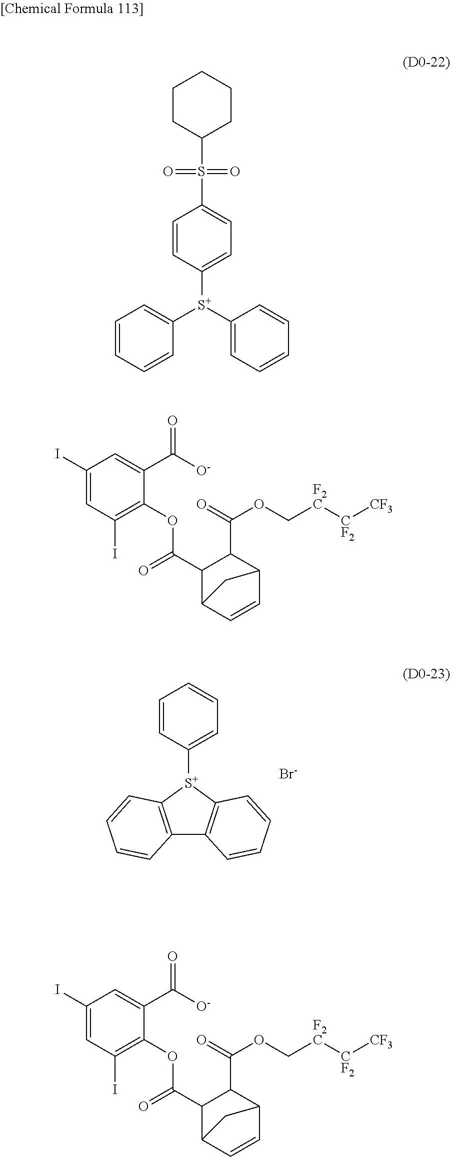 [SMILES: O=S(=O)(c1ccc([S+](c2ccccc2)c2ccccc2)cc1)C1CCCCC1 ; O=C([O-])c1cc(I)cc(I)c1OC(=O)C1C2C=CC(C2)C1C(=O)OCC(F)(F)C(F)(F)C(F)(F)F ; c1ccc(-[s+]2c3ccccc3c3ccccc32)cc1 ; [Br-] ; O=C([O-])c1cc(I)cc(I)c1OC(=O)C1C2C=CC(C2)C1C(=O)OCC(F)(F)C(F)(F)C(F)(F)F]

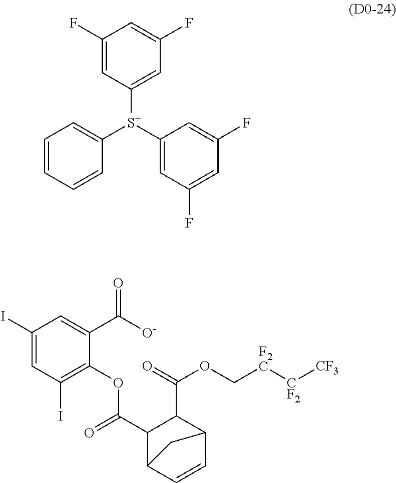 [SMILES: Fc1cc(F)cc([S+](c2ccccc2)c2cc(F)cc(F)c2)c1 ; O=C([O-])c1cc(I)cc(I)c1OC(=O)C1C2C=CC(C2)C1C(=O)OCC(F)(F)C(F)(F)C(F)(F)F]

### <Preparation of Resist Composition>

### Examples 1 to 30 and Comparative Examples 1 to 4

Each of the components shown in Tables 1 to 4 was mixed and dissolved to prepare a resist composition of each Example.

TABLE 1

| | Component | Component | Component | Component |
|---|---|---|---|---|
| | (A) | (B) | (D) | (S) |
| Comparative | (A)-1 | (B)-1 | (D)-31 | (S)-1 |
| Example 1 | [100] | [20.0] | [5.0] | [8000] |
| Comparative | (A)-1 | (B)-1 | (D)-32 | (S)-1 |
| Example 2 | [100] | [20.0] | [9.7] | [8000] |
| Comparative | (A)-1 | (B)-1 | (D)-33 | (S)-1 |
| Example 3 | [100] | [20.0] | [7.3] | [8000] |
| Comparative | (A)-1 | (B)-1 | (D)-34 | (S)-1 |
| Example 4 | [100] | [20.0] | [6.8] | [8000] |

TABLE 2

| | Component | Component | Component | Component |
|---|---|---|---|---|
| | (A) | (B) | (D) | (S) |
| Example | (A)-1 | (B)-1 | (D)-1 | (S)-1 |
| 1 | [100] | [20.0] | [6.7] | [8000] |
| Example | (A)-1 | (B)-1 | (D)-2 | (S)-1 |
| 2 | [100] | [20.0] | [6.9] | [8000] |
| Example | (A)-1 | (B)-1 | (D)-3 | (S)-1 |
| 3 | [100] | [20.0] | [7.8] | [8000] |
| Example | (A)-1 | (B)-1 | (D)-4 | (S)-1 |
| 4 | [100] | [20.0] | [7.8] | [8000] |
| Example | (A)-1 | (B)-1 | (D)-5 | (S)-1 |
| 5 | [100] | [20.0] | [8.3] | [8000] |
| Example | (A)-1 | (B)-1 | (D)-6 | (S)-1 |
| 6 | [100] | [20.0] | [8.1] | [8000] |
| Example | (A)-1 | (B)-1 | (D)-7 | (S)-1 |
| 7 | [100] | [20.0] | [8.5] | [8000] |
| Example | (A)-1 | (B)-1 | (D)-8 | (S)-1 |
| 8 | [100] | [20.0] | [9.0] | [8000] |
| Example | (A)-1 | (B)-1 | (D)-9 | (S)-1 |
| 9 | [100] | [20.0] | [9.0] | [8000] |
| Example | (A)-1 | (B)-1 | (D)-10 | (S)-1 |
| 10 | [100] | [20.0] | [9.4] | [8000] |
| Example | (A)-1 | (B)-1 | (D)-11 | (S)-1 |
| 11 | [100] | [20.0] | [9.9] | [8000] |
| Example | (A)-1 | (B)-1 | (D)-12 | (S)-1 |
| 12 | [100] | [20.0] | [10.8] | [8000] |

TABLE 3

| | Component | Component | Component | Component |
|---|---|---|---|---|
| | (A) | (B) | (D) | (S) |
| Example | (A)-1 | (B)-1 | (D)-13 | (S)-1 |
| 13 | [100] | [20.0] | [9.0] | [8000] |
| Example | (A)-1 | (B)-1 | (D)-14 | (S)-1 |
| 14 | [100] | [20.0] | [9.1] | [8000] |
| Example | (A)-1 | (B)-1 | (D)-15 | (S)-1 |
| 15 | [100] | [20.0] | [9.0] | [8000] |
| Example | (A)-1 | (B)-1 | (D)-16 | (S)-1 |
| 16 | [100] | [20.0] | [9.0] | [8000] |
| Example | (A)-1 | (B)-1 | (D)-17 | (S)-1 |
| 17 | [100] | [20.0] | [10.0] | [8000] |
| Example | (A)-1 | (B)-1 | (D)-18 | (S)-1 |
| 18 | [100] | [20.0] | [9.6] | [8000] |
| Example | (A)-1 | (B)-1 | (D)-19 | (S)-1 |
| 19 | [100] | [20.0] | [9.2] | [8000] |
| Example | (A)-1 | (B)-1 | (D)-20 | (S)-1 |
| 20 | [100] | [20.0] | [10.1] | [8000] |
| Example | (A)-1 | (B)-1 | (D)-21 | (S)-1 |
| 21 | [100] | [20.0] | [8.7] | [8000] |
| Example | (A)-1 | (B)-1 | (D)-22 | (S)-1 |
| 22 | [100] | [20.0] | [10.3] | [8000] |
| Example | (A)-1 | (B)-1 | (D)-23 | (S)-1 |
| 23 | [100] | [20.0] | [9.0] | [8000] |
| Example | (A)-1 | (B)-1 | (D)-24 | (S)-1 |
| 24 | [100] | [20.0] | [9.6] | [8000] |

TABLE 4

| | Component | Component | Component | Component |
|---|---|---|---|---|
| | (A) | (B) | (D) | (S) |
| Example | (A)-1 | (B)-2 | (D)-9 | (S)-1 |
| 25 | [100] | [12.6] | [9.0] | [8000] |
| Example | (A)-1 | (B)-2 | (D)-13 | (S)-1 |
| 26 | [100] | [12.6] | [9.0] | [8000] |
| Example | (A)-1 | (B)-2 | (D)-24 | (S)-1 |
| 27 | [100] | [12.6] | [9.6] | [8000] |
| Example | (A)-2 | (B)-1 | (D)-24 | (S)-1 |
| 28 | [100] | [20.0] | [9.6] | [8000] |
| Example | (A)-3 | (B)-1 | (D)-24 | (S)-1 |
| 29 | [100] | [20.0] | [9.6] | [8000] |
| Example | (A)-3 | (B)-2 | (D)-24 | (S)-1 |
| 30 | [100] | [12.6] | [9.6] | [8000] |

In Tables 1 to 4, each abbreviation has the following meaning. The numerical values in the brackets are blending amounts (parts by mass).

(A)-1: polymer compound represented by Chemical Formula (A1-1) The weight-average molecular weight (Mw) of the polymer compound (A1-1) in terms of standard polystyrene determined by GPC measurement was 5800, and the polydispersity (Mw/Mn) thereof was 1.54. The copolymerization compositional ratio (the ratio (molar ratio) of each constitutional unit in the structural formula) determined by ¹C-NMR was 1/m=50/50.

(A)-2: polymer compound represented by Chemical Formula (A1-2) The weight-average molecular weight (Mw) of the polymer compound (A1-2) in terms of standard polystyrene determined by GPC measurement was 6000, and the polydispersity (Mw/Mn) thereof was 1.55. The copolymerization compositional ratio (the ratio (molar ratio) of each constitutional unit in the structural formula) determined by ¹³C-NMR was 1/m=50/50.

(A)-3: polymer compound represented by Chemical Formula (A1-3) The weight-average molecular weight (Mw) of the polymer compound (A1-3) in terms of standard polystyrene determined by GPC measurement was 5900, and the polydispersity (Mw/Mn) thereof was 1.54. The copolymerization compositional ratio (the ratio (molar ratio) of each constitutional unit in the structural formula) determined by ¹³C-NMR was 1/m=50/50.

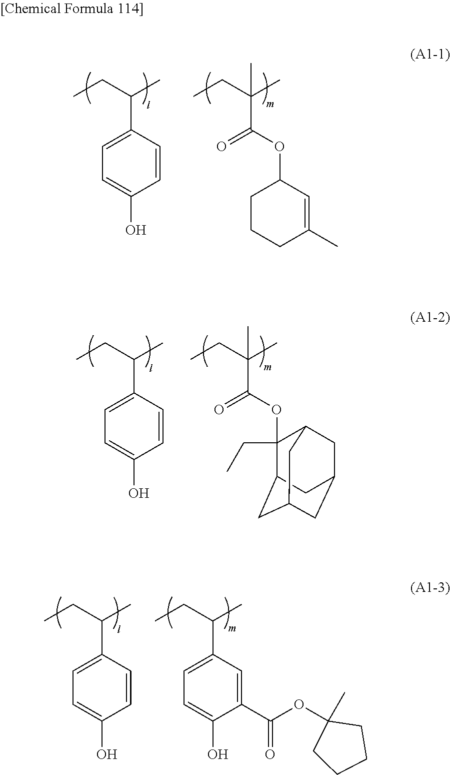

(B)-1: acid generator formed of compound (B1-1) shown below
(B)-2: acid generator formed of compound (B1-2) shown below

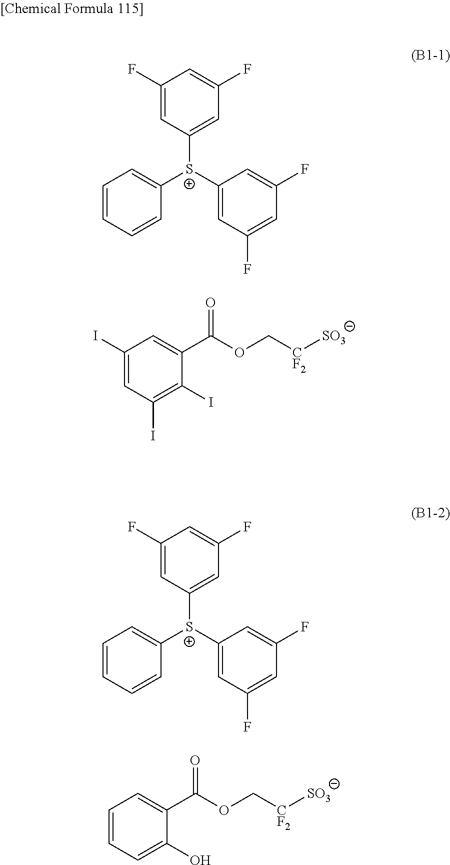

(D)-1 to (D)-24: compounds (D0-1) to (D0-24) shown above
(D)-31: acid diffusion control agent formed of compound (D1-1) shown below
(D)-32: acid diffusion control agent formed of compound (D1-2) shown below
(D)-33: acid diffusion control agent formed of compound (D1-3) shown below
(D)-34: acid diffusion control agent formed of compound (D1-4) shown below

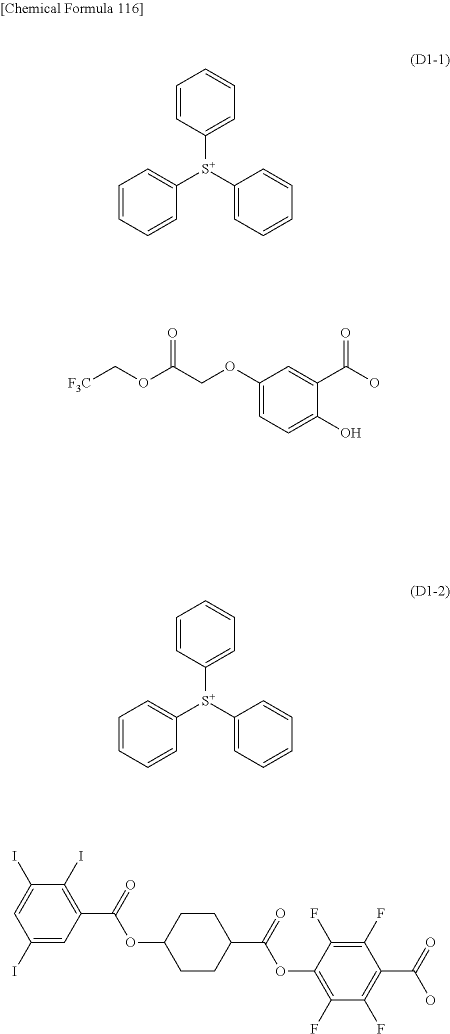 [SMILES: c1ccc([S+](c2ccccc2)c2ccccc2)cc1 ; O=C(COc1ccc(O)c(C(=O)O)c1)OCC(F)(F)F ; c1ccc([S+](c2ccccc2)c2ccccc2)cc1 ; O=C(OC1CCC(C(=O)Oc2c(F)c(F)c(C(=O)O)c(F)c2F)CC1)c1cc(I)cc(I)c1I]

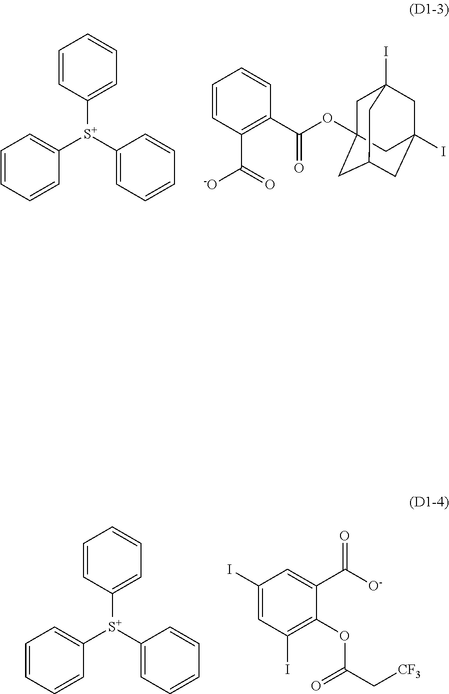

(S)-1: mixed solvent of propylene glycol monomethyl ether acetate/propylene glycol monomethyl ether at mass ratio of 60/40

### <Resist Pattern Formation>

The resist composition of each example was applied onto an 8-inch silicon substrate which had been subjected to a hexamethyldisilazane (HMDS) treatment using a spinner, the coated film was subjected to a pre-bake (PAB) treatment on a hot plate at a temperature of 110° C. for 60 seconds so that the coated film was dried to form a resist film having a film thickness of 50 nm.

Next, drawing (light exposure was performed on the resist film by using an electron beam lithography apparatus JEOL-JBX-9300FS (manufactured by JEOL Ltd.) at an acceleration voltage of 100 kV such that the target size was a 1:1 line-and-space pattern (hereinafter referred to as “LS pattern”) having a line width of 50 nm.

Thereafter, a postexposure bake (PEB) treatment was carried out on the resist film at 100° C. for 60 seconds.

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.). Thereafter, water rinsing was performed for 15 seconds using pure water.

As a result, a 1:1 LS pattern having a line width of 50 nm was formed.

### [Evaluation of Sensitivity]

An optimum exposure amount Eop (μC/cm²) at which the LS pattern with a target size was formed according to the method in the section of <Resist pattern formation> was determined. The results thereof are listed in the columns of “Eop (μC/cm²)” in Tables 5 and 6.

### [Evaluation of Line Width Roughness (LWR)]

3σ, which is a measure indicating LWR, was determined for the LS pattern formed by <Resist pattern formation> described above.

Here, “3σ” denotes the triple value (3σ) (unit: nm) of the standard deviation (σ) determined from measurement results obtained by measuring 400 sites of line positions in the longitudinal direction of the line with a scanning electron microscope (trade name: S-9380, manufactured by Hitachi High-Tech Corporation, acceleration voltage of 800 V). The results thereof are listed in the columns of “LWR (nm)” in Tables 5 and 6.

In a case where the value of the 3c is small, this indicates that the roughness of a line side wall is small and an LS pattern with a uniform width is obtained.

### [Evaluation of Pattern Shape]

In the <Resist pattern formation>, the cross-sectional shape of the line in each of the obtained LS patterns was observed with a scanning electron microscope (trade name: SU8000, manufactured by Hitachi High-Tech Corporation, acceleration voltage of 300 V). In the observation of the cross-sectional shape of the line, the shape was observed at any 20 points of the line pattern.

A line width (Lt) in an upper portion of the line and a line width (Lm) in an intermediate portion of the line were measured in the height direction of the LS pattern, and the ratio (Lt/Lm) thereof was determined from the average value of 20 measured values of each of Lt and Lm. The rectangularity of the cross-sectional shape of the line denotes that the rectangularity is more satisfactory (that is, the rectangularity is high) as the value of Lt/Lm is close to 1.

In regard to the pattern shape, the rectangularity of the cross-sectional shape of the line was evaluated according to the following evaluation criteria using the ratio (Lt/Lm) as an index.

The evaluation was performed such that a case where 0.95≤(Lt/Lm)≤1.05 was satisfied was evaluated as “A: particularly satisfactory”; a case where 0.90≤(Lt/Lm)<0.95 or 1.05<(Lt/Lm)≤1.10 is satisfied is evaluated as “B: satisfactory”; and a case where (Lt/Lm)<0.90 or 1.10<(Lt/Lm) was satisfied was evaluated as “C: defective”.

As the evaluation of the pattern shape, the values of Lt/Lm and the evaluation results of rectangularity are listed in Tables 5 and 6.

TABLE 5

| | PAB | PEB | Eop | LWR | Pattern shape | |
|---|---|---|---|---|---|---|
| | (° C.) | (° C.) | (μC/cm²) | (nm) | Lt/Lm | Rectangularity |
| Comparative | 110 | 100 | 139 | 6.5 | 0.94 | B |
| Example 1 | | | | | | |
| Comparative | 110 | 100 | 92 | 6.1 | 0.87 | C |
| Example 2 | | | | | | |
| Comparative | 110 | 100 | 96 | 6.2 | 0.84 | C |
| Example 3 | | | | | | |
| Comparative | 110 | 100 | 97 | 6.4 | 0.89 | C |
| Example 4 | | | | | | |
| Example 1 | 110 | 100 | 106 | 5.7 | 0.90 | B |
| Example 2 | 110 | 100 | 104 | 5.4 | 0.94 | B |
| Example 3 | 110 | 100 | 104 | 5.2 | 0.98 | A |
| Example 4 | 110 | 100 | 98 | 5.6 | 0.90 | B |
| Example 5 | 110 | 100 | 98 | 5.4 | 0.91 | B |
| Example 6 | 110 | 100 | 98 | 5.3 | 0.94 | B |
| Example 7 | 110 | 100 | 97 | 5.1 | 0.97 | A |
| Example 8 | 110 | 100 | 97 | 5.2 | 0.94 | B |
| Example 9 | 110 | 100 | 97 | 5.1 | 0.99 | A |
| Example 10 | 110 | 100 | 97 | 5.2 | 1.03 | A |
| Example 11 | 110 | 100 | 97 | 5.4 | 1.06 | B |
| Example 12 | 110 | 100 | 97 | 5.6 | 1.10 | B |

TABLE 6

| | PAB | PEB | Eop | LWR | Pattern shape | |
|---|---|---|---|---|---|---|
| | (° C.) | (° C.) | (μC/cm²) | (nm) | Lt/Lm | Rectangularity |
| Example 13 | 110 | 100 | 97 | 5.2 | 0.99 | A |
| Example 14 | 110 | 100 | 96 | 5.0 | 1.01 | A |
| Example 15 | 110 | 100 | 99 | 5.4 | 0.94 | B |
| Example 16 | 110 | 100 | 99 | 5.5 | 0.92 | B |
| Example 17 | 110 | 100 | 97 | 5.1 | 0.98 | A |
| Example 18 | 110 | 100 | 97 | 5.3 | 0.98 | A |
| Example 19 | 110 | 100 | 92 | 5.4 | 0.92 | B |
| Example 20 | 110 | 100 | 92 | 5.2 | 0.98 | A |
| Example 21 | 110 | 100 | 102 | 5.3 | 0.96 | A |
| Example 22 | 110 | 100 | 98 | 5.2 | 0.98 | A |
| Example 23 | 110 | 100 | 98 | 5.2 | 0.98 | A |
| Example 24 | 110 | 100 | 96 | 5.1 | 1.01 | A |
| Example 25 | 110 | 100 | 103 | 5.1 | 0.99 | A |
| Example 26 | 110 | 100 | 104 | 5.2 | 0.99 | A |
| Example 27 | 110 | 100 | 103 | 5.1 | 0.99 | A |
| Example 28 | 110 | 100 | 96 | 5.0 | 0.99 | A |
| Example 29 | 110 | 100 | 95 | 4.9 | 0.99 | A |
| Example 30 | 110 | 100 | 102 | 5.1 | 0.99 | A |

As shown in the results listed in Tables 5 and 6, based on the comparison between Examples 1 to 30 and Comparative Examples 1 to 4, it can be confirmed that the resist compositions of Examples 1 to 30 to which the present invention was applied achieved both high sensitivity and roughness reduction and were capable of forming a pattern having a satisfactory shape with high rectangularity.

It can be also confirmed that the resist compositions of Examples 1 to 30 had a higher effect of reducing the roughness than that of the resist compositions of Comparative Examples 1 to 4.

Hereinbefore, preferred examples of the present invention have been described, but the present invention is not limited to these examples. Additions, omissions, substitutions, and other modifications can be made without departing from the scope of the present invention. The present invention is not limited by the description above, but is limited only by the appended claims.

What is claimed is:

1. A resist composition which generates an acid upon light exposure and whose solubility in a developing solution is changed by an action of the acid, the resist composition comprising:
a base material component (A) whose solubility in a developing solution is changed by the action of the acid; and
a compound (D0) represented by General Formula (d0), 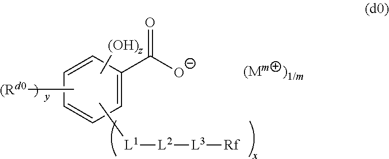
wherein Rf represents a hydrocarbon group which may have a fluorine atom, L² represents a divalent cyclic group which may have a substituent, L¹ and L³ each independently represents a divalent linking group, x represents an integer of 1 to 4, when x represents an integer of 2 or greater, a plurality of (-L¹-L²-L³-Rf)'s may be the same as or different from each other, R^{d0} represents an iodine atom or a bromine atom, y represents an integer of 1 to 4, when y represents an integer of 2 or greater, a plurality of R^{d0}'s may be the same as or different from each other, z represents an integer of 0 to 3, wherein 2≤x+y+z≤5 is satisfied, (M^{m+})_{1/m} represents a counter cation, and m represents an integer of 1 or greater.

2. The resist composition according to claim 1, wherein in Formula (d0), Rf represents a fluorinated hydrocarbon group.

3. The resist composition according to claim 1, wherein in Formula (d0), a total number of fluorine atoms contained in x pieces of Rf's is 5 or more and 9 or less.

4. The resist composition according to claim 1, wherein in Formula (d0), y represents 2 or 3.

5. A resist pattern formation method comprising:
forming a resist film on a support using the resist composition according to claim 1;
exposing the resist film to light; and
developing the resist film exposed to light to form a resist pattern.

6. The resist pattern formation method according to claim 5, wherein the resist film is exposed to an extreme ultraviolet ray or an electron beam in exposing the resist film to light.

7. A compound which is represented by General Formula (d0), 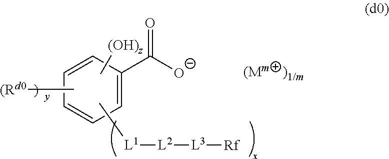
wherein Rf represents a hydrocarbon group which may have a fluorine atom, L² represents a divalent cyclic group which may have a substituent, L¹ and L³ each independently represents a divalent linking group, x represents an integer of 1 to 4, when x represents an integer of 2 or greater, a plurality of (-L¹-L²-L³-Rf)'s may be the same as or different from each other, R^{d0} represents an iodine atom or a bromine atom, y represents an integer of 1 to 4, when y represents an integer of 2 or greater, a plurality of R^{d0}'s may be the same as or different from each other, z represents an integer of 0 to 3, wherein 2≤x+y+z≤5 is satisfied, (M^{m+})_{1/m} represents a counter cation, and m represents an integer of 1 or greater.

8. An acid diffusion control agent comprising the compound according to claim 7.
